(19) United States
(12) Patent Application Publication
(10) Pub. No.: US 2017/0298402 A1
(43) Pub. Date: Oct. 19, 2017

(54) SELF-LABELING NUCLEIC ACIDS AND METHODS OF USE

(71) Applicant: President and Fellows of Harvard College, Cambridge, MA (US)

(72) Inventors: David R. Liu, Lexington, MA (US); Richard McDonald, Somerville, MA (US)

(73) Assignee: President and Fellows of Harvard College, Cambridge, MA (US)

(21) Appl. No.: 15/516,092

(22) PCT Filed: Oct. 2, 2015
(86) PCT No.: PCT/US15/53764

§ 371 Date: Mar. 31, 2017

Related U.S. Application Data
(60) Provisional application No. 62/059,642, filed on Oct. 3, 2014.

(51) Int. Cl. C12P 19/34; C12Q 1/68; G01N 33/50; G01N 33/58; C12Q 1/00

(52) U.S. Cl. CPC C12P 19/34; C12Q 1/6806; C12Q 1/6816; C12Q 1/6841; G01N 33/5011; G01N 33/582; C12Q 1/00; C12Q 2563/107; G01N 33/50; C12Q 2525/101

(57) ABSTRACT

Some aspects of this disclosure relate to the identification of reactive nucleic acids, such as, for example, reactive cellular RNAs. Minimal reactive motif consensus sequences and isolated reactive nucleic acids conforming to such consensus sequences are provided herein. Reactive probes that selectively form covalent bonds with reactive nucleic acids are also provided herein, as are methods of using such probes, for example, for labeling, identification, characterization, and/or quantification of reactive nucleic acids. This disclosure further provides fusion molecules comprising a reactive nucleic acid conjugated to a heterologous molecule of interest, such as a heterologous nucleic acid or protein. Methods and kits for generating and using such fusion proteins to track, detect, and/or quantify molecules of interest in a biological sample are also provided as are expression constructs encoding fusion molecules of reactive nucleic acids and heterologous nucleic acids of interest.

### RELATED APPLICATIONS

The present application claims priority under 35 U.S.C. §119(e) to U.S. provisional application, U.S. Ser. No. 62/059,642, filed Oct. 3, 2014, the entire contents of which is incorporated herein by reference.

### GOVERNMENT SUPPORT

This invention was made with Government support under GM065865 and GM099359 awarded by National Institutes of Health (NIH). The Government has certain rights in the invention.

### BACKGROUND

Recent data suggesting that most of the genome is transcribed into functional, non-coding RNA,¹ together with an increasing awareness of the complexity of RNA post-transcriptional regulation, have created the need for new tools to study and discover bioactive RNAs.^{2,3} Our knowledge of naturally occurring catalytic RNAs is limited to the ribosome and ten classes of phosphodiester-hydrolyzing RNAs.^{4,5} The two types of chemical reactions catalyzed by known biological ribozymes represent a small fraction of the impressive suite of transformations catalyzed by laboratory-evolved RNAs.⁶

Chemical tools to discover novel functional RNAs lag behind the wealth of approaches available to investigate the proteome. Probes exploiting the ubiquity of unusually nucleophilic functional groups have found widespread use in activity-based protein profiling (ABPP)^{7,8} and self-labeling fusion proteins that are now widely used to study and manipulate proteins.⁹⁻¹⁴ ABPP uses electrophilic small-molecule “activity-based probes”, typically based on previously discovered irreversible enzyme inhibitors, linked to an affinity tag such as biotin. Enzymes that react with these probes are isolated and identified using the affinity tag.

Two recent reports described the use of SELEX to isolate synthetic RNAs that react with α-halo acetamides,^{15,16} which have been used as mechanism-based inhibitors of serine proteases. The development and use of activity-based probes to identify naturally occurring, unusually reactive RNAs, however, remains unexplored. Current tools for studying RNA function instead rely predominantly on non-covalent binding between an RNA and its corresponding ligand or receptor.¹⁷⁻²⁰

### SUMMARY

Some aspects of this disclosure provide methods for identifying and detecting reactive nucleic acids, such as, for example, reactive cellular RNAs. Some aspects of this disclosure relate to the development of probes that form covalent bonds with reactive nucleic acids based on their chemical reactivity. Such probes are useful for the labeling, identification, characterization, and/or quantification of naturally-occurring reactive nucleic acids.

Some aspects of this disclosure relate to the discovery of unusually nucleophilic RNAs within the transcriptome. Some aspects of this disclosure relate to the surprising discovery that such unusually nucleophilic RNA species can be selectively labeled with electrophilic probes. Some aspects of this disclosure relate to the development of electrophilic probes designed and optimized to react with unusually nucleophilic nucleic acids, such as unusually nucleophilic RNA or DNAs. Some aspects of this disclosure relate to the delineation and optimization of minimal nucleic acid sequences required for nucleophilic reactivity. Some aspects of this disclosure relate to the identification and optimization of minimal reactive nucleic acid sequences comprising a stem-bulge-stem-loop structure with a nucleophilic guanosine (G) residue within the stem-bulge structure.

Nucleic acids comprising minimal reactive sequences are also provided herein, as are methods and systems of using such reactive sequences, for example, as self-labeling tags that can be conjugated to a molecule of interest. The reactive nucleic acid sequences provided herein are thus useful as self-labeling tags that can be conjugated to molecules of interest to enable detecting, quantifying, and/or tracking the tagged molecules of interest. Systems and methods for identifying, detecting, quantifying, and/or tracking molecules of interest in vivo or in vitro are also provided herein.

Some aspects of this disclosure provide fusion molecules comprising minimal reactive nucleic acid sequence(s) conjugated to a molecule of interest. The molecule of interest in a fusion molecule provided herein is heterologous to the minimal reactive nucleic acid sequence in the fusion molecules provided herein, for example, in that the molecule of interest does not naturally comprise the respective minimal reactive nucleic acid sequence(s) of the fusion molecule, and does not exist in nature in a form conjugated to the minimal reactive nucleic acid sequence(s). Suitable molecules of interest that can be conjugated to reactive nucleic acid sequences provided herein include, but are not limited to, nucleic acids, proteins, polysaccharides, lipids, lipoproteins, metabolites, and small molecules.

Some aspects of this disclosure provide methods and reagents for generating fusion molecules comprising a reactive nucleic acid sequence provided herein and a heterologous molecule. In addition, some aspects of this disclosure provide methods and reagents for conjugating a reactive nucleic acid sequence provided herein to a reactive probe. In some embodiments, the reactive probe comprises a detectable label or a reactive handle, such as a click-chemistry moiety, that can be used to detect or to further chemically modify the labeled fusion molecule. The methods provided herein are thus useful, for example, for the labeling, detection, quantification, and/or tracking of fusion molecules comprising a reactive nucleic acid sequence and a molecule of interest.

Some aspects of this disclosure provide electrophilic probes capable of forming a covalent bond with a reactive RNA, for example, with an RNA comprising the consensus sequence of SEQ ID NO: 1. In some embodiments, the probe comprises a disubstituted epoxide. In some embodiments, the disubstituted epoxide is a 2,3-disubstituted epoxide. In some embodiments, the epoxide is a cis-epoxide.

In some embodiments, the electrophilic probe is of Formula (F):

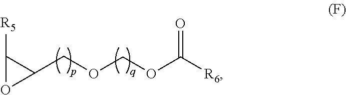

wherein p is an integer between 1 and 10, inclusive; q is an integer between 1 and 10, inclusive; R⁵ and/or R⁶ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{T}; —CO₂R^{T}; —CN; —SCN; —SR^{T}; —SOR^{T}; —SO₂R^{T}; —NO₂; —N(R^{T})₂; —NHC(O)R^{T}; or —C(R^{T})₃; wherein each occurrence of R^{T} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R⁵ and/or R⁶ is, independently, [CH]ₙ—R^{TT}; wherein n is an integer between 0 and 25, e.g., 0, 1, 2, 3, 4, 5, 6, 7, 8, 9, 10, 11, 12, 13, 14, 15, 16, 17, 18, 19, 20, 21, 22, 23, 24, or 25; and R^{TT} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{U}; —CO₂R^{U}; —CN; —SCN; —SR^{U}; —SOR^{U}; —SO₂R^{U}; —NO₂; —N(R^{U})₂; —NHC(O)R^{U}; or —C(R^{U})₃; wherein each occurrence of R^{U} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, R⁵ is CH₃. In some embodiments, p is 1, 2, 3, 4, 5, 6, 7, 8, 9, or 10. In some embodiments, q is 1, 2, 3, 4, 5, 6, 7, 8, 9, or 10. In some embodiments, p and/or q is, independently, an integer between 2 and 10, inclusive; between 2 and 5, inclusive; or between 2 and 4, inclusive. In some embodiments, p is 3 and q is an integer between 2 and 10, inclusive; between 2 and 5, inclusive; or between 2 and 4, inclusive. In some embodiments, q is 2 and p is an integer between 2 and 10, inclusive; between 2 and 5, inclusive; or between 2 and 4, inclusive. In some embodiments, p is 3 and q is 2.

In some embodiments, C5 and C6 are in cis.

In some embodiments, the disubstituted epoxide is of Formula 1

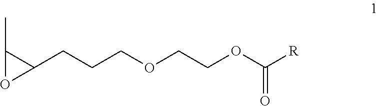

wherein R is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —OR^{A}; —CN; —SCN; —SR^{A}; —SOR^{A}; —SO₂R^{A}; —NO₂; —N(R^{A})₂; —NHC(O)R^{A}; or —C(R^{A})₃;
wherein each occurrence of R^{A} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R is —[CH]ₙ—R^{AA}; wherein

n is an integer between 0 and 25; and
R^{AA} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —OR^{B}; —C(═O)R^{B}; —CO₂R^{B}; —CN; —SCN; —SR^{B}; —SOR^{B}; —SO₂R^{B}; —NO₂; —N(R^{B})₂; —NHC(O)R^{B}; or —C(R^{B})₃;
wherein each occurrence of R^{B} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R comprises a detectable label. In some embodiments, the detectable label comprises a binding agent, a fluorescent or bioluminescent moiety, an enzyme, a ligand, a sequence tag, a radioactive isotope, or a mass tag. In some embodiments, the detectable label comprises biotin. In some embodiments, R comprises a click chemistry handle. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene. In some embodiments, the disubstituted epoxide is the compound of any one of Formula 9-16.

Some aspects of this disclosure provide fusion molecules comprising (a) a nucleic acid comprising a reactive G nucleotide (G*) in a 5′-WGAG*RN₄₋₃₀AGGC[U/T]CR-3′ (SEQ ID NO: 1) nucleotide sequence, wherein R represents A or G; W represents A, or [U/T]; [U/T] represents U or T; and N represents any nucleotide; and (b) a heterologous molecule conjugated to the nucleic acid of (a).

In some embodiments, the nucleic acid of (a) comprises a stem-loop structure. In some embodiments, the nucleic acid of (a) comprises a stem-bulge structure. In some embodiments, the nucleotide sequence of (a) comprises ribonucleotides. In some embodiments, [U/T] represents U. In some embodiments, the nucleotide sequence of (a) comprises deoxyribonucleotides. In some embodiments, [U/T] represents T.

In some embodiments, N₄₋₃₀ comprises 4-30, 4-25, 4-20, 4-15, 4-10, 6-30, 10-30, 15-30, or 20-30 nucleotides. In some embodiments, N₄₋₃₀ comprises 4, 5, 6, 7, 8, 9, 10, 11, 12, 13, 14, 15, 16, 17, 18, 19, 20, 21, 22, 23, 24, or 25, nucleotides. In some embodiments, N₄₋₃₀ comprises an even number of nucleotides. In some embodiments, N₄₋₃₀ represents a nucleotide sequence forming a stem-loop structure. In some embodiments, the stem comprises two G-C base pairs. In some embodiments, the stem comprises two or more G-C or C-G base pairs. In some embodiments, the nucleotide at the 5′-end of N₄₋₃₀ is a G or a C and forms a G-C or a C-G base pair with the nucleotide at the 3′-end of N₄₋₃₀. In some embodiments, the third nucleotide from the 5′-end of N₄₋₃₀ is a G or a C and forms a G-C or a C-G base pair with the third nucleotide from the 3′-end of N₄₋₃₀.

In some embodiments, N₄₋₃₀ comprises a 5′-GCGC[U/T]N₁₀AGGGC-3′ sequence (SEQ ID NO: 2). In some embodiments, the nucleic acid of (a) comprises a 5′-WGAG*GGCCC[U/T]N₁₋₃G[U/T]A[U/T]GRAAGGGC[U/T]AGGC[U/T]C-3′ sequence (SEQ ID NO: 3). In some embodiments, the nucleic acid of (a) comprises a 5′-GGCAAAGAG*GGCCC[U/T]N₁₋₃G[U/T]A[U/T]GRAAGGGC[U/T]AGGC [U/T]CR[U/T][U/T]G[U/T]-3′ sequence (SEQ ID NO: 4).

In some embodiments, the nucleic acid of (a) comprises a 5′-GGCAAAGAG*GGCCC[U/T]GGGG[U/T]A[U/T]GGAAGGGC[U/T]AGGC[U/T]CG[U/T][U/T]G[U/T]-3′ sequence (SEQ ID NO: 5). In some embodiments, the nucleic acid of (a) comprises a 5′-GGCAAAGAG*GGCCC[U/T]AG[U/T]A[U/T]GAAAGGGC[U/T]AGGC[U/T]CA[U/T][U/T]G[U/T]-3′ sequence (SEQ ID NO: 6). In some embodiments, the nucleic acid of (a) comprises a 5′-GGCCGCUCCAGAAGAGGGCCCCUUCGGGGGCUAGGCUCGAUGUCGGCC-3′ sequence (SEQ ID NO: 7).

In some embodiments, the fusion molecule comprises multiple copies of the nucleic acid of (a), thus providing multiple reactive G nucleotides that can be bound by multiple electrophilic probes, resulting in a higher labeling density per molecule of interest. In some embodiments, the fusion molecule comprises 2-10 copies of the nucleic acid of (a). In some embodiments, the fusion molecule comprises 3 copies of the nucleic acid of (a).

In some embodiments, the nucleic acid of (a) is conjugated to the heterologous molecule of (b) via a covalent bond. In some embodiments, the nucleic acid of (a) is conjugated to the heterologous molecule of (b) via a linker. In some embodiments, the linker is a cleavable linker. In some embodiments, the heterologous molecule of (b) is conjugated to the 5′-end of the nucleic acid of (a). In some embodiments, the heterologous molecule of (b) is conjugated to the 3′-end of the nucleic acid of (a). In some embodiments, the heterologous molecule of (b) is a heterologous nucleic acid. In some embodiments, the heterologous molecule of (b) is a heterologous RNA. In some embodiments, the RNA is a non-coding RNA. In some embodiments, the RNA encodes a protein. In some embodiments, the heterologous molecule of (b) is a protein, a polysaccharide, or a small molecule. In some embodiments, the heterologous molecule of (b) is a binding agent. In some embodiments, the binding agent is a ligand.

In some embodiments, the reactive G nucleotide (G*) is covalently bound to a nucleophilic moiety. In some embodiments, the nucleophilic moiety is a disubstituted epoxide. In some embodiments, the disubstituted epoxide is a 2,3-disubstituted epoxide. In some embodiments, the epoxide is a cis-epoxide.

In some embodiments, the activity-based electrophilic probe comprises or consists of a structure of Formula (F):

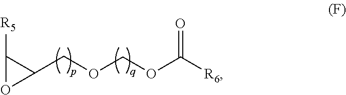

wherein p is an integer between 1 and 10, inclusive; q is an integer between 1 and 10, inclusive; R⁵ and/or R⁶ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{T}; —CO₂R^{T}; —CN; —SCN; —SR^{T}; —SOR^{T}; —SO₂R^{T}; —NO₂; —N(R^{T})₂; —NHC(O)R^{T}; or —C(R^{T})₃; wherein each occurrence of R^{T} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R⁵ and/or R⁶ is, independently, [CH]ₙ—R^{TT}; wherein n is an integer between 0 and 25, e.g., 0, 1, 2, 3, 4, 5, 6, 7, 8, 9, 10, 11, 12, 13, 14, 15, 16, 17, 18, 19, 20, 21, 22, 23, 24, or 25; and R^{TT} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{U}; —CO₂R^{U}; —CN; —SCN; —SR^{U}; —SOR^{U}; —SO₂R^{U}; —NO₂; —N(R^{U})₂; —NHC(O)R^{U}; or —C(R^{U})₃; wherein each occurrence of R^{U} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, R⁵ is CH₃. In some embodiments, p is 1, 2, 3, 4, 5, 6, 7, 8, 9, or 10. In some embodiments, q is 1, 2, 3, 4, 5, 6, 7, 8, 9, or 10. In some embodiments, p and/or q is, independently, an integer between 2 and 10, inclusive; between 2 and 5, inclusive; or between 2 and 4, inclusive. In some embodiments, p is 3 and q is an integer between 2 and 10, inclusive; between 2 and 5, inclusive; or between 2 and 4, inclusive. In some embodiments, q is 2 and p is an integer between 2 and 10, inclusive; between 2 and 5, inclusive; or between 2 and 4, inclusive. In some embodiments, p is 3 and q is 2.

In some embodiments, C5 and C6 are in cis.

In some embodiments, the disubstituted epoxide is of Formula 1

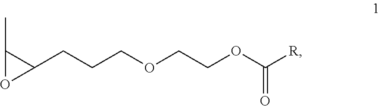

wherein R is as defined for Formula 1 above.

In some embodiments, R comprises a detectable label. In some embodiments, the detectable label comprises a binding agent, a fluorescent or bioluminescent moiety, an enzyme, a ligand, a sequence tag, a radioactive isotope, or a mass tag. In some embodiments, the detectable label comprises biotin. In some embodiments, the detectable label comprises a fluorescent or bioluminescent moiety.

In some embodiments, the disubstituted epoxide is the compound of any one of Formula 9-16 as provided in FIG. 3.

Some aspects of this disclosure provide methods for detecting a molecule of interest, wherein the method comprises (a) providing a fusion molecule comprising (i) the molecule of interest; and (ii) a nucleic acid conjugated to the molecule of interest of (i), wherein the nucleic acid comprises a reactive G nucleotide (G*) in a 5′-WGAG*RN₄₋₃₀AGGC[U/T]CR-3′ (SEQ ID NO: 1) nucleotide sequence, wherein R represents A or G; W represents A or [U/T]; [U/T] represents U or T; and N represents any nucleotide; (b) contacting the molecule of (a) with a disubstituted epoxide comprising a detectable label under conditions suitable for the disubstituted epoxide to form a covalent bond to the reactive G nucleotide (G*); and (c) detecting the detectable label bound to the molecule of interest.

In some embodiments, the method further comprises contacting a cell or tissue with the fusion molecule of (a). In some embodiments, the contacting and/or detecting is in vitro. In some embodiments, the contacting and/or detecting is in vivo. In some embodiments, the method further comprises administering the fusion molecule of (a) to a subject. In some embodiments, the method comprises administering the fusion molecule of (a) bound to the disubstituted epoxide of (b) to the subject.

In some embodiments, the nucleic acid of (a)(ii) comprises a stem-loop structure. In some embodiments, the nucleic acid of (a)(ii) comprises a stem-bulge structure. In some embodiments, the nucleotide sequence of (a)(ii) comprises ribonucleotides. In some embodiments, [U/T] represents U. In some embodiments, the nucleotide sequence of (a)(ii) comprises deoxyribonucleotides. In some embodiments, [U/T] represents T.

In some embodiments, N₄₋₃₀ comprises 4-30, 4-25, 4-20, 4-15, 4-10, 6-30, 10-30, 15-30, or 20-30 nucleotides. In some embodiments, N₄₋₃₀ comprises 4, 5, 6, 7, 8, 9, 10, 11, 12, 13, 14, 15, 16, 17, 18, 19, 20, 21, 22, 23, 24, or 25, nucleotides. In some embodiments, N₄₋₃₀ comprises an even number of nucleotides. In some embodiments, N₄₋₃₀ represents a nucleotide sequence forming a stem-loop structure. In some embodiments, the stem comprises two G-C base pairs. In some embodiments, the stem comprises two or more G-C or C-G base pairs. In some embodiments, the nucleotide at the 5′ end of N₄₋₃₀ is a G or a C and forms a G-C or a C-G base pair with the nucleotide at the 3′ end of N₄₋₃₀. In some embodiments, the third nucleotide from the 5′ end of N₄₋₃₀ is a G or a C and forms a G-C or a C-G base pair with the third nucleotide from the 3′ end of N₄₋₃₀. In some embodiments, N₄₋₃₀ comprises a 5′-GCCC[U/T]N₁₀AGGGC-3′ sequence (SEQ ID NO: 2).

In some embodiments, the nucleic acid of (a)(ii) comprises a 5′-WGAG*GGCCC[U/T]N₁₋₃G[U/T]A[U/T]GRAAGGGC[U/T]AGGC[U/T]C-3′ sequence (SEQ ID NO: 3). In some embodiments, the nucleic acid of (a)(ii) comprises a 5′-GGCAAAGAG*GGCCC[U/T]N₁₋₃G[U/T]A[U/T]GRAAGGGC[U/T]AGGC[U/T]CR[U/T][U/T]G[U/T]-3′ sequence (SEQ ID NO: 4). In some embodiments, the nucleic acid of (a) comprises a 5′-GGCAAAGAG*GGCCC[U/T]GGGG[U/T]A[U/T]GGAAGGGC[U/T]AGGC[U/T]CG[U/T][U/T]G[U/T]-3′ sequence (SEQ ID NO: 5). In some embodiments, the nucleic acid of (a) comprises a 5′-GGCAAAGAG*GGCCC[U/T]AG[U/T]A[U/T]GAAAGGGC[U/T]AGGC[U/T]CA[U/T][U/T]G[U/T]-3′ sequence (SEQ ID NO: 6). In some embodiments, the nucleic acid of (a) comprises a 5′-GGCCGCUCCAGAAGAGGGCCCCUUCGGGGGCUAGGCUCGAUGUCGGCC-3′ sequence (SEQ ID NO: 7).

In some embodiments, the fusion molecule comprises multiple copies of the nucleic acid of (a)(ii). In some embodiments, the fusion molecule comprises 2-10 copies of the nucleic acid of (a)(ii). In some embodiments, the fusion molecule comprises 3 copies of the nucleic acid of (a)(ii).

In some embodiments, the nucleic acid of (a)(ii) is conjugated to the molecule of interest via a covalent bond. In some embodiments, the nucleic acid of (a)(ii) is conjugated to the molecule of interest via a linker. In some embodiments, the linker is a cleavable linker. In some embodiments, the molecule of interest is conjugated to the 5′ end of the nucleic acid of (a)(ii). In some embodiments, the molecule of interest is conjugated to the 3′ end of the nucleic acid of (a)(ii).

In some embodiments, the molecule of interest is a nucleic acid. In some embodiments, the molecule of interest is an RNA. In some embodiments, the RNA is a non-coding RNA. In some embodiments, the RNA encodes a protein. In some embodiments, the molecule of interest is a protein, a polysaccharide, a small molecule, or a metabolite. In some embodiments, the molecule of interest is a binding agent. In some embodiments, the binding agent is a ligand.

In some embodiments, the disubstituted epoxide is a 2,3-disubstituted epoxide. In some embodiments, the disubstituted epoxide is a cis-epoxide. In some embodiments, the detectable label comprises a binding agent, a fluorescent or bioluminescent moiety, a sequence tag, a radioactive isotope, or a mass tag.

Some aspects of this disclosure provide kits for detecting a molecule of interest, the kit comprising (a) a nucleic acid comprising or encoding a reactive G nucleotide (G*) in a 5′-WGAG*RN₄₋₃₀AGGC[U/T]CR-3′ (SEQ ID NO: 1) nucleotide sequence, wherein R represents A or G; W represents A or [U/T]; [U/T] represents U or T; and N represents any nucleotide; and (b) reagents for conjugating the nucleic acid of (a) to a molecule of interest.

In some embodiments, the kit further comprises an electrophilic probe for labeling the reactive G nucleotide (G*). In some embodiments, the electrophilic probe comprises a disubstituted epoxide conjugated to a detectable label. In some embodiments, the disubstituted epoxide is a 2,3-disubstituted epoxide. In some embodiments, the disubstituted epoxide comprises an ester moiety. In some embodiments, the epoxide is a cis-epoxide. In some embodiments, the detectable label comprises a binding agent, a fluorescent or bioluminescent moiety, a sequence tag, a radioactive isotope, a mass tag, or a click chemistry handle. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, the kit comprises an expression construct comprising a nucleic acid sequence encoding the nucleic acid sequence of (a) and a cloning site for inserting a heterologous nucleic acid sequence encoding a gene product of interest to generate a hybrid nucleic acid sequence encoding a fusion of the nucleic acid sequence of (a) and the gene product of interest. In some embodiments, the gene product of interest is an RNA. In some embodiments, the RNA is a non-coding RNA. In some embodiments, the gene product of interest is a binding agent. In some embodiments, the gene product of interest is an aptamer.

Some aspects of this disclosure provide expression constructs comprising (a) a nucleic acid sequence encoding a reactive nucleic acid; and (b) a cloning site for inserting a heterologous nucleic acid sequence encoding a gene product of interest to generate a hybrid nucleic acid sequence encoding a fusion of the nucleic acid sequence of (a) and the gene product of interest. In some embodiments, the reactive nucleic acid of (a) is a reactive nucleic acid provided herein, for example, a reactive nucleic acid comprising a sequence that conforms to the consensus sequence provided herein as SEQ ID NO: 1. In some embodiments, the gene product of interest is an RNA. In some embodiments, the gene product of interest is a coding RNA. In some embodiments, the gene product of interest is a binding agent. In some embodiments, the gene product of interest is an aptamer.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1. Electrophilic probes for the discovery of unusually nucleophilic RNA. Panel a: Electrophilic small molecules with carefully tailored reactivity selectively react with unusually nucleophilic RNAs. The reacted RNA species can be isolated using an affinity handle such as biotin linked to the probe. Panel b: Electrophiles were assayed by LC/MS for their ability to react irreversibly with an RNA pool of random sequence. Electrophiles capable of forming products with random RNA sequences, as evaluated by mass spectrometry, were deemed too reactive, and variants with attenuated reactivity were identified. An excessively electrophilic candidate probe (top) and an attenuated version (bottom) are shown. Panel c: Eight electrophiles of tuned electrophilicity that can serve as probes to identify unusually reactive RNAs. R=biotin.

FIG. 2. A catalytic RNA from the A. pernix genome that reacts with a disubstituted epoxide. Panel a: PAGE streptavidin gel mobility shift following incubation of random sequence RNA (“random”), the A. pernix species from Round 6 of the selection (“selected”), and the A. pernix fragment corresponding to the reference genome sequence (“genomic”) with epoxide probe 1 (1 mM) for 16 hours at room temperature (1 μM RNA). The complete gel is shown in FIG. 16. Panel b: Secondary structure model of the minimized A. pernix catalytic RNA. The reactive guanosine (G⁹) was identified by RNAse T1 digestion and mass spectrometry. Panel c: Sequence logo based on high-throughput DNA sequencing of the RNA species surviving selection of a partially randomized RNA pool derived from the minimized 42-nt A. pernix catalytic RNA.

FIG. 3. Epoxide substrate selectivity of the catalytic RNA. Panel a: Epoxide analogues were tested to determine substrate specificity of the A. pernix catalytic RNA. Modification was analyzed by LC/MS following digestion into mononucleotides by nuclease P1. Relative reaction efficiencies, shown in parentheses, were determined by comparing the ion counts of unmodified GMP to modified GMP; all values are relative to the reaction efficiency of epoxide 9 (defined as 1.00). R=—(CH₂)₅NHBoc. Panel b: Reaction efficiency of the A. pernix catalytic RNA (1 μM) with trans-epoxide 1 (1.3 mM) over time. Time-course data show mean values±s.d. for three replicates. Panel c: Epoxide probes that react efficiently with the optimized catalytic RNA as determined by LC/MS. Panel d: For azide-epoxide 14 and TAMRA-epoxide 16, RNA modification was also visualized by fluorescence imaging (λₑₓ=532 nm, λₑₘ=580 nm) following PAGE. Lane 1: background reactivity or binding of the DBCO-TAMRA probe with the catalytic RNA. Lane 2: incubation with azide-epoxide 14 followed by copper-free click reaction with DBCO-TAMRA. Lane 3: incubation with TAMRA-epoxide 16. The complete gel is shown in FIG. 17.

FIG. 4. Application of the epoxide-opening catalytic RNA to enrich RNAs of interest from total cellular RNA and to capture RNA-binding proteins. Panel a: Transcriptional fusion of a self-labeling catalytic RNA to an RNA of interest may enable selective, covalent RNA modification in a complex biological sample. Panel b: Total RNA from HEK 293T cells was reacted with epoxide-azide 14, followed by DBCO-TAMRA. Total RNA was analyzed by PAGE, and TAMRA-modified RNAs were visualized by fluorescence imaging. Lanes 1 and 6: in vitro transcribed catalytic RNA-fused 5S rRNA containing one or three copies of the catalytic RNA, respectively, rather than cellular RNA. Lanes 2 and 3: the inactive C9-G35 mutant RNA. Lanes 4-8: 5S rRNA fused to one copy (lanes 4-5) or three copies (lanes 6-8) of the active optimized catalytic RNA. Bands at the top of the gel result from incomplete removal of excess DBCO-TAMRA probe or background labeling of cellular rRNAs/mRNAs. The complete gel is shown in FIG. 18. Panel c: Western blot probing the presence of three known ASH1 mRNA-binding proteins (Puf6, Khd1, and She2) and one non-binding protein control (Guk1) in yeast cell lysate. Lanes 1 and 2: Lysate incubated overnight with streptavidin-coated magnetic beads only (lane 1) or pre-incubated with 5 μg of epoxide 1-modified ASH1-catalytic RNA (lane 2). Unbound proteins were washed away, and captured proteins were eluted at 95° C. Lane 3: Input lysate prior to incubation with beads. The complete gel is shown in FIG. 19.

FIG. 5. Agarose gel electrophoresis of cDNA following 1 and 5 rounds of selection. The Round 1 material was amplified for 20 cycles, and Round 5 was amplified for 15 cycles. The smallest band in the molecular weight ladder corresponds to 50 bp, and each higher band differs by 50 bp.

FIG. 6. In vitro selection leads to the identification of genome-encoded RNA species that react with epoxide 1. Panel a: In vitro selection cycle of genome-encoded RNAs capable of reacting with electrophilic, biotinylated probes. Electrophilic groups are represented as small circles, biotin groups are shown as diamonds, and streptavidin is represented as a large circle binding to streptavidin. Panel b: The post-round 6 RNA pool was incubated with each of the electrophilic probes and a streptavidin gel mobility shift assay was performed to identify the reactive electrophile(s). Lane 1 shows the RNA band following incubation with an unreactive ketone substrate. Lane 2 reveals that RNA species (arrow) within the RNA pool react with disubstituted epoxide probe 1. The complete gel is shown in FIG. 15. Panel c: Following incubation with the epoxide probe, the RNA pool was digested into mononucleotides by nuclease P1 and analyzed by LC/MS. Guanosine nucleotides, but not A, U, or C nucleotides, modified with epoxide 1 were observed.

FIG. 7. A. pernix-based epoxide-opening catalytic RNA sequences. Panel a: The A. pernix sequence emerging from round 6 of the selection that was tested for reactivity with epoxide probe 1. Panel b: The corresponding A. pernix reference genome sequence.

FIG. 8. Minimization of the A. pernix ribozyme. Progressive truncations from the 5′- and 3′-termini revealed a 42-nt transcript that effectively catalyzes epoxide ring opening.

FIG. 9. Kinetic characterization of the minimized A. pernix ribozyme. Panel a: Product formation for the reaction between the ribozyme (1 μM) and biotin-epoxide 1 (1.3 mM). Panel b: Michaelis-Menten curves to determine k_{cat} and Kₘ (5 hr reaction time, 1 μM RNA). Formation of product was calculated as the mean of three independent experiments from streptavidin gel mobility shift assays. Michaelis-Menten parameter calculations were corrected to account for 47% of the RNA folding into a reactive conformation. Data represents mean values±s.d. for three replicates.

FIG. 10. Sequence requirements of the A. pernix ribozyme. Panel a: The partially randomized A. pernix library used for reselection. Panel b: Structure-activity relationships inferred from site-directed mutation studies. Panel c: Based on the results from reselection of the partially randomized A. pernix library and site-directed mutation studies, we developed a reactive minimal motif for bioinformatic searching. The motif requires three unspecified base pairs followed by a loop ranging from 4-25-nt in length.

FIG. 11. Sequence, structure, and reactivity of the optimized ribozyme. Panel a: Product formation is shown for the reaction between the ribozyme (1 μM) and biotin-epoxide 1 (1.3 mM). Data represents mean values±s.d. for three replicates. Panel b: Sequence and secondary structure model of the optimized epoxide-opening catalytic RNA. The grey-colored stem nucleotides are derived from the tRNA scaffold.

FIG. 12. The effect of monovalent and divalent salts on reaction efficiency. Ribozyme-epoxide reactivity as a function of Panel a: NaCl concentration, Panel b: KCl concentration, and Panel c: MgCl₂ concentration.

FIG. 13. Mass spectral (MS) characterization of the epoxide-ribozyme reaction. Negative-ion mode MS/MS of the epoxide-GMP product from nuclease P1 digestion of the ribozyme (m/z=620.285) reveals ions corresponding to epoxide-purine (m/z=408.258) and the unmodified ribose (m/z=211.019). An additional fragment resulting from hydrolysis of the ester group in the probe (m/z=310.176) is also observed. The proposed structures show bond formation between N7 of guanosine and the less-hindered carbon of the epoxide, although the data are consistent with either or both epoxide carbon atoms undergoing bond formation.

FIG. 14. Structural elucidation of the epoxide-guanosine product. Panel a: LC comparison of the GMP-epoxide authentic product and digested ribozyme product. Panel b: MS/MS fragmentation comparison of the authentic and digested ribozyme products.

FIG. 15. Complete gel from FIG. 6, Panel b. The dashed box highlights the portion of the gel shown in the figure.

FIG. 16. Complete gel from FIG. 2, Panel a. The dashed box highlights the portion of the gel shown in the figure.

FIG. 17. Complete gel from FIG. 3, Panel d. The dashed box highlights the portion of the gel shown in the figure.

FIG. 18. Complete gel from FIG. 4, Panel b. Panel a: The dashed box highlights the portion of the gel shown in the figure. Panel b: Following fluorescence imaging, the gel was stained with the general RNA stain SYBR Green II and imaged for total RNA content.

FIG. 19. Complete Western blot from FIG. 4, Panel c. The portions of the Western blot corresponding to Puf6 (Panel a), Khd1 (Panel b), She2 (Panel c), and Gud1 (Panel d) portions of the figure are shown in the dashed boxes.

### DEFINITIONS

### Chemical Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic,” as used herein, includes both saturated and unsaturated, straight chain (i.e., unbranched), branched, acyclic, and cyclic (i.e., carbocyclic) hydrocarbons, which are optionally substituted with one or more functional groups. It is understood from the above description that the term “aliphatic,” whether preceded by the terms substituted or unsubstituted, and unless otherwise specified, encompasses “cyclic or acyclic” and “branched or unbranched” groups. As will be appreciated by one of ordinary skill in the art, “aliphatic” is intended herein to include, but is not limited to, alkyl, alkenyl, alkynyl, and carbocyclyl (cycloalkyl, cycloalkenyl, and cycloalkynyl) moieties. In certain embodiments, as used herein, “aliphatic” is used to indicate those aliphatic groups (cyclic, acyclic, substituted, unsubstituted, branched or unbranched) having 1-20 carbon atoms. Unless otherwise specified, each instance of an aliphatic group is independently unsubstituted or substituted with one or more substituents, as valency permits, and which results in a stable compound. Exemplary substituents are further described herein.

The term “alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), and the like, which may bear one or more substituents. Additional examples of alkyl groups include n-heptyl (C₇), n-octyl (C₈) and the like, which may bear one or more substituents. Unless otherwise specified, each instance of an alkyl group is independently unsubstituted or substituted with one or more substituents, as valency permits, and which results in a stable compound. Exemplary substituents are further described herein.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

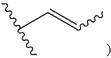

may be an (E)- or (Z)-double bond.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroaliphatic,” as used herein, refers to an aliphatic moiety, as defined herein, which includes both saturated and unsaturated, nonaromatic, straight chain (i.e., unbranched), branched, acyclic or cyclic (i.e., heterocyclic) groups which are optionally substituted with one or more substituents, and which contain one or more oxygen, sulfur, nitrogen, phosphorus, or silicon atoms, e.g., in place of carbon atoms. It is understood from the above description that the term “heteroaliphatic,” whether preceded by the terms substituted or unsubstituted, and unless otherwise specified, encompasses “cyclic or acyclic” and “branched or unbranched” groups. It is also understood, similar to aliphatic, that “heteroaliphatic” is intended to encompass heteroalkyl, heteroalkenyl, heteroalkynyl, and heterocyclic (heterocycloalkyl, heterocycloalkenyl, and heterocycloalkynyl) moieties. The terms “heteroalkyl,” “heteroalkenyl,” and “heteroalkynyl” are defined similarly, i.e., respectively refer to an alkyl, alkenyl, and alkynyl group, as defined herein, which are optionally substituted with one or more substituents, and which contain one or more oxygen, sulfur, nitrogen, phosphorus, or silicon atoms, e.g., in place of carbon atoms. Unless otherwise specified, each instance of a heteroaliphatic group is independently unsubstituted or substituted with one or more substituents, as valency permits, and which results in a stable compound. Exemplary substituents are further described herein.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl. In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur. Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted or substituted with one or more substituents, as valency permits, and which results in a stable compound. Exemplary substituents are further described herein.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

The term “acyl,” as used herein, refers to a group having the general formula —C(═O)R^{X5}, —C(═O)OR^{X5}, —C(═O)SR^{X5}, —C(═O)N(R^{X6})₂, —C(═NR^{X6})R^{X1}, —C(═NR^{X6})OR^{X5}, —C(═NR^{X6})SR^{X5}, —C(═NR^{X6})N(R^{X6})₂, —C(═S)R^{X5}, —C(═S)OR^{X5}, —C(═S)SR^{X5}, and —C(═S)N(R^{X6})₂, wherein each occurrence of R^{X5} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{X6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two R^{X6} groups are joined to form an substituted or unsubstituted heterocyclic ring.

Any one of the groups described herein is independently optionally substituted unless expressly provided otherwise. “Optionally substituted” refers to a group which may be substituted or unsubstituted. In general, the term “substituted” means that at least one hydrogen present on a group (e.g., a carbon or nitrogen atom) is replaced with a permissible substituent, e.g., a substituent which upon substitution results in a stable moiety or compound, e.g., a compound which does not spontaneously undergo transformation such as by rearrangement, cyclization, elimination, or other reaction, and preferably possess stability sufficient to allow manufacture, and which maintains its integrity for a sufficient period of time to be useful for the purposes detailed herein. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms may have hydrogen substituents and/or any substituent as described herein which satisfy the valencies of the heteroatom and results in the formation of a stable moiety.

Exemplary substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, and combinations thereof, e.g., aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). Other exemplary substituents are further described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}—P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “protecting group” as used herein, refers to a chemical modification of a functional group of a compound that prevents the functional group to take part in an undesired chemical reaction. Protecting groups play an important role in multi-step organic compound synthesis, and suitable protecting groups for various functional groups and chemical environments are well known in the art. Examples of protecting groups are nitrogen protecting groups, oxygen protecting groups, sulfur protecting groups, and carboxylic acid protecting groups are described in more detail herein.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, F⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, ethanoate, propanoate, benzoate, glycerate, lactate, tartrate, glycolate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻).

These and other exemplary substituents and protecting groups are described in more detail in the Detailed Description, Examples, and claims. The invention is not intended to be limited in any manner by the above exemplary listing of substituents and protecting groups.

### Other Definitions

The term “amino acid,” as used herein, includes any naturally occurring and non-naturally occurring amino acid. Suitable natural and non-natural amino acids will be apparent to the skilled artisan, and include, but are not limited to, those described in S. Hunt, The Non-Protein Amino Acids: In Chemistry and Biochemistry of the Amino Acids, edited by G. C. Barrett, Chapman and Hall, 1985. Some non-limiting examples of non-natural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine. In the context of amino acid sequences, “X” or “Xaa” represents any amino acid residue, e.g., any naturally occurring and/or any non-naturally occurring amino acid residue.

The term “antibody,” as used herein, refers to a protein belonging to the immunoglobulin superfamily. The terms antibody and immunoglobulin are used interchangeably. Antibodies from any mammalian species (e.g., human, mouse, rat, goat, pig, horse, cattle, camel) and from non-mammalian species (e.g., from non-mammalian vertebrates, birds, reptiles, amphibia) are within the scope of the term. Suitable antibodies and antibody fragments for use in the context of some embodiments of the present invention include, for example, human antibodies, humanized antibodies, domain antibodies, F(ab′), F(ab′)2, Fab, Fv, Fc, and Fd fragments, antibodies in which the Fc and/or FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; and antibodies in which the FR and/or CDR1 and/or CDR2 regions have been replaced by homologous human or non-human sequences. In some embodiments, so-called single chain antibodies (e.g., ScFv), (single) domain antibodies, and other intracellular antibodies may be used in the context of the present invention. Domain antibodies, camelid and camelized antibodies and fragments thereof, for example, VHH domains, or nanobodies, such as those described in patents and published patent applications of Ablynx NV and Domantis are also encompassed in the term antibody. Further, chimeric antibodies, e.g., antibodies comprising two antigen-binding domains that bind to different antigens, are also suitable for use in the context of some embodiments of the present invention.

The term “binding agent,” as used herein refers to any molecule that binds another molecule. In some embodiments, a binding agent binds another molecule with high affinity. In some embodiments, a binding agent binds another molecule with high specificity. The binding agent may be a protein, peptide, nucleic acid, polysaccharide, polymer, or small molecule. Examples for binding agents include, without limitation, antibodies, antibody fragments, receptors, ligands, aptamers, and adnectins.

The term “conjugating,” and the terms “conjugated,” and “conjugation” refer to an association of two entities, for example, of two molecules such as a reactive nucleic acid and a molecule of interest (e.g., a heterologous nucleic acid, a protein, a polysaccharide, a lipid, lipoprotein, a metabolite, or a small molecule). The association can be, for example, via a direct or indirect (e.g., via a linker) covalent linkage or via non-covalent interactions. In some embodiments, the association is via a covalent bond. In some embodiments, two molecules are conjugated via a linker connecting both molecules. For example, in some embodiments where a reactive nucleic acid is conjugated to a molecule of interest to form a fusion molecule, the two molecules may be conjugated, for example, via a phosphodiester bond, a peptide bond, a C—C bond, a C—O bond, a C—S bond. In some embodiments, two molecules, e.g., a reactive nucleic acid and a molecule of interest, are conjugated directly with no intervening sequence or molecular structure separating the two molecules. For example, a reactive nucleic acid may be conjugated to a nucleic acid of interest via cloning the two nucleic acids into a single expression construct and expressing them as a single fusion molecule, thus creating a transcript comprising both the sequence of the reactive nucleic acid and the nucleic acid of interest. Similarly, if the molecule of interest is not a nucleic acid, a covalent fusion may be generated by chemical synthesis methods attaching the reactive nucleic acid to the molecule of interest, e.g., via click chemistry. In some embodiments, a reactive nucleic acid is associated with a molecule of interest indirectly via a linker. In some embodiments, the linker is a cleavable linker. Two molecules, for example, a reactive nucleic acid and a molecule of interest, may also be conjugated to one another through non-covalent interactions (e.g., electrostatic interactions). For example, in some embodiments, one molecule may comprise a ligand moiety that binds to a receptor moiety comprised in the second molecule to form a complex. For example, a molecule of interest may be generated that comprises a binding agent, such as, e.g., an aptamer-binding moiety, and the reactive nucleic acid may be generated in a form comprising a sequence that is bound by the binding agent, e.g., an aptamer. Molecules that are conjugated to one another, either directly or via one or more additional moieties that serve as a linking agent, form a structure that is sufficiently stable so that the molecules remain physically associated under the conditions in which the structure is used, e.g., under physiological conditions. For example, if a fusion molecule of a molecule of interest and a reactive nucleic acid is used to detect the location of the fusion molecule in a target tissue, the fusion will be designed in a manner that ensures that the molecule of interest and the reactive nucleic acid remain attached to each other for a time long enough to allow detection under conditions typically encountered within the target tissue.

The term “consensus sequence,” as used herein in the context of nucleic acid sequences, refers to a sequence representing the most frequent nucleotide residues found at each position in a plurality of similar sequences, for example, and a plurality of sequences sharing the same general structure or function. Typically, a consensus sequence is determined by sequence alignment in which similar sequences are compared to each other and similar sequence motifs are calculated. In some embodiments, a consensus sequence does not exist in nature. In some embodiments, a consensus sequence is generated in silico based on the alignment of several similar sequences, e.g., of several homologous sequences. Suitable methods and algorithms for determining a consensus sequence are provided herein and additional suitable sequences will be apparent to those of skill in the art. The disclosure is not limited in this respect. In the context of reactive nucleic acids, a consensus sequence of a reactive nucleic acid sequence may, in some embodiments, be a sequence that can react with a given activity-based probe.

The term “detectable label” or “label” refers to a moiety that has at least one element, isotope, or functional group incorporated into the moiety which enables detection of the compound to which the label is attached. In general, a label can fall into any one (or more) of five classes: 1) a label which contains isotopic moieties, which may be radioactive or heavy isotopes, including, but not limited to, ²H, ³H, ¹³C, ¹⁴C, ¹⁵N, ³¹P, ³²P, ³⁵S, ⁶⁷Ga, ^{99m}Tc (Tc-99m), ¹¹¹In, ¹²³I, ¹²⁵I, ¹⁶⁹Yb, and ¹⁸⁶Re; 2) a label which contains an immune moiety, which may be antibodies or antigens, which may be bound to enzymes (e.g., such as horseradish peroxidase); 3) a label which is a colored, luminescent, phosphorescent, or fluorescent moieties (e.g., such as FITC, GFP, quantum dots, etc.); 4) a label which has one or more photoaffinity moieties; and 5) a label which has a ligand moiety with one or more known binding partners (such as biotin-streptavidin, FK506-FKBP, etc.). In certain embodiments, a label comprises a radioactive isotope, preferably an isotope which emits detectable particles, such as β particles. In certain embodiments, the label comprises a fluorescent moiety. In certain embodiments, the label is the fluorescent label fluorescein-isothiocyanate (FITC). In certain embodiments, the label comprises a ligand moiety with one or more known binding partners. In certain embodiments, the label comprises biotin, which may be detected using a streptavidin conjugate (e.g., fluorescent streptavidin conjugates such as Streptavidin ALEXA FLUOR® 568 conjugate (SA-568) and Streptavidin ALEXA FLUOR® 800 conjugate (SA-800), Invitrogen). In some embodiments, a label is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising fluorophores that emit light of different colors such as red, yellow, and cyan fluorescent proteins. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols Methods of biochemical analysis, v. 47 Wiley-Interscience, Hoboken, N.J., 2006; and Chudakov, D M, et al., Physiol Rev. 90(3):1103-63, 2010, for discussion of GFP and numerous other fluorescent or luminescent proteins. In some embodiments, a label comprises a dark quencher, e.g., a substance that absorbs excitation energy from a fluorophore and dissipates the energy as heat. In certain embodiments, the label comprises one or more photoaffinity moieties for the direct elucidation of intermolecular interactions in biological systems. A variety of known photophores can be employed, for example, photophores relying on photoconversion of diazo compounds, azides, or diazirines to nitrenes or carbenes (see, Bayley, H., Photogenerated Reagents in Biochemistry and Molecular Biology (1983), Elsevier, Amsterdam, the entire contents of which are incorporated herein by reference). In certain embodiments of the invention, the photoaffinity labels employed are o-, m-, and p-azidobenzoyls, substituted with one or more halogen moieties, including, but not limited to 4-azido-2,3,5,6-tetrafluorobenzoic acid. In certain embodiments, the label comprises one or more fluorescent moieties such as a fluorescent small molecule or a fluorescent protein. In some embodiments, the label comprises a quantum dot. In certain embodiments, the label comprises a ligand moiety with one or more known binding partners. In certain embodiments, the label comprises the ligand moiety biotin. In certain embodiments, the label is an imaging agent. Exemplary imaging agents include, but are not limited to, those used in positron emissions tomography (PET), computer assisted tomography (CAT), single photon emission computerized tomography, x-ray, fluoroscopy, and magnetic resonance imaging (MRI); anti-emetics; and contrast agents. Exemplary diagnostic agents include but are not limited to, fluorescent moieties, luminescent moieties, magnetic moieties; gadolinium chelates (e.g., gadolinium chelates with DTPA, DTPA-BMA, DOTA and HP-DO3A), iron chelates, magnesium chelates, manganese chelates, copper chelates, chromium chelates, iodine-based materials useful for CAT and x-ray imaging, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹mTc, ¹⁴C, ¹³N, ¹⁵O, ³²P, ³³P, and ¹⁸F. Fluorescent and luminescent moieties include, but are not limited to, a variety of different organic or inorganic small molecules commonly referred to as “dyes,” “labels,” or “indicators.” Examples include, but are not limited to, fluorescein, rhodamine, acridine dyes, Alexa dyes, cyanine dyes, etc. Numerous fluorescent and luminescent dyes and proteins are known in the art (see, e.g., U.S. Patent Publication 2004/0067503; Valeur, B., “Molecular Fluorescence: Principles and Applications,” John Wiley and Sons, 2002; and Handbook of Fluorescent Probes and Research Products, Molecular Probes, 9^{th} edition, 2002). In some embodiments, a detectable label may be a mass tag, for example, a molecule of known mass and ion decay pattern that can be identified in a mass analysis assay, such as mass spectrometry.

The term “fusion molecule” refers to a molecule comprising a plurality of heterologous molecules that are conjugated to each other via a covalent bond. In certain embodiments, a fusion molecule as provided herein comprises a reactive nucleic acid that is covalently bound to a molecule of interest (e.g., a heterologous nucleic acid, a protein, a polysaccharide, lipid, lipoprotein, a metabolite, or a small molecule). In some embodiments, a fusion molecule comprises a linker connecting the individual, heterologous molecules. In some embodiments, the linker is a cleavable linker. For example, a fusion molecule of a reactive nucleic acid as provided herein and a nucleic acid of interest may comprise, in some embodiments, a single nucleic acid molecule comprising both the sequence of the reactive nucleic acid and the sequence of the nucleic acid of interest, either with or without any additional nucleotides or sequences separating the two. A linker sequence may separate the two molecules, and the linker may comprise a cleavage site, such as a UV-sensitive cleavage site or a nuclease cleavage site. For another example, a fusion molecule of a reactive nucleic acid as provided herein and a molecule of interest that is not a nucleic acid (e.g., a protein, small molecule, or metabolite of interest) may comprise, in some embodiments, a single molecule comprising both the sequence of the reactive nucleic acid and the molecule of interest, covalently attached to each other either with or without any additional chemical moieties (e.g., nucleotides or nucleic acid sequences, amino acids or amino acid sequences, or synthetic polymers) separating the two. A linker sequence may separate the two molecules, and the linker may comprise a cleavage site, such as a UV-sensitive cleavage site or a protease cleavage site. The exemplary fusion molecules provided herein are not meant to limit the scope of the disclosure and additional suitable configurations of fusion molecules will be apparent to those of skill in the art based on the instant disclosure.

The term “linker,” as used herein, refers to a chemical group or a molecule linking two molecules or moieties, e.g., a supercharged protein and a nuclease. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker is an organic molecule, group, polymer, or chemical moiety. In some embodiments, the linker is a cleavable linker, e.g., the linker comprises a bond that can be cleaved upon exposure to a cleaving activity, such as UV light or a hydrolytic enzyme, such as a lysosomal protease. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker comprises repeats of the tri-peptide Gly-Gly-Ser, e.g., comprising the sequence (GGS)ₙ, wherein n represents at least 1, 2, 3, 4, 5, 6, 7, 8, 9, 10, or more repeats. In some embodiments, the linker comprises the sequence (GGS)₆ (SEQ ID NO:8). In some embodiments, the peptide linker is the 16 residue “XTEN” linker, or a variant thereof (See, e.g., Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 9), SGSETPGTSESA (SEQ ID NO: 10), or SGSETPGTSESATPEGGSGGS (SEQ ID NO: 11). In some embodiments, the peptide linker is one or more selected from VPFLLEPDNINGKTC (SEQ ID NO: 12), GSAGSAAGSGEF (SEQ ID NO: 13), SIVAQLSRPDPA (SEQ ID NO: 14), MKIIEQLPSA (SEQ ID NO: 15), VRHKLKRVGS (SEQ ID NO: 16), GHGTGSTGSGSS (SEQ ID NO: 17), MSRPDPA (SEQ ID NO: 18); or GGSM (SEQ ID NO: 19).

The term “molecule of interest” refers to any molecule that can be conjugated to a reactive nucleic acid provided herein. Accordingly, a molecule of interest may be, without limitation, a nucleic acid, a protein, a polysaccharide, a lipid, lipoprotein, a metabolite, or a small molecule. A molecule of interest may include, in some embodiments, a molecule encoded by the genome of a subject. In some embodiments, a molecule of interest is a therapeutic agent. In some embodiments, a molecule of interest is a diagnostic agent. In some embodiments, a molecule of interest is a binding agent. In some embodiments, a molecule of interest includes a reactive moiety, for example, a click chemistry functional group.

The term “non-naturally occurring,” as used herein in the context of fusion molecules or molecules of interest, refers to the fact that the respective molecule, by virtue of its origin or manipulation does not occur in nature or is conjugated to a heterologous molecule that it is not linked to in nature, wherein the conjugation results in a characteristic of the conjugate that is distinct from the characteristic that the non-conjugated, or naturally-occurring molecule exhibits.

The term “nucleic acid molecule” and the term “nucleic acid” as used interchangeably herein, refer to a compound comprising a nucleoside, a nucleotide, or a polymer of nucleotides. Typically, polymeric nucleic acids, e.g., nucleic acids comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid. On the other hand, a nucleic acid may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “pharmaceutical composition,” as used herein, refers to a composition that can be administrated to a subject, for example, in the context of treatment or diagnosis of a disease or disorder or in the context of conducting scientific experiments. In some embodiments, a pharmaceutical composition comprises an active ingredient, e.g., a fusion molecule comprising a reactive nucleic acid and a molecule of interest, and a pharmaceutically acceptable excipient. Typically, a pharmaceutical composition comprises an effective amount of the active ingredient, for example, an amount sufficient to effect a biological change in the subject, including, but not limited to, a detectable change in the level of a labeled molecule in a cell, tissue, or body fluid of the subject, or an amelioration of an undesired phenotype, for example, of a symptom of a disease that's this subject may exhibit.

The term “protein” is interchangeably used herein with the terms “peptide” and “polypeptide” and refers to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a (poly)saccharide group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, an effector domain and a binding domain. A protein may be a binding agent, for example, an antibody or antibody fragment, a ligand, or a receptor, or a binding fragment of any of these. A protein may be an enzyme that catalyzes a reaction. A protein may also be a structural protein, a chaperone, or a transport protein. Other suitable proteins in addition to those described herein will be apparent to those of skill in the art based on the instant disclosure. The disclosure is not limited in this respect.

The term “reactive handle,” as used herein, refers to a reactive moiety that can partake in a bond-forming reaction under physiological conditions. Reactive handles can be used to conjugate entities with reactive handles that can react with each other. Examples of suitable reactive handles are, for example, chemical moieties that can partake in a click chemistry reaction (see, e.g., H. C. Kolb, M. G. Finn and K. B. Sharpless (2001). Click Chemistry: Diverse Chemical Function from a Few Good Reactions. Angewandte Chemie International Edition 40 (11): 2004-2021). Some suitable reactive handles are described herein and additional suitable reactive handles will be apparent to those of skill in this art, as the present disclosure is not limited in this respect.

The term “small molecule” and the term “organic compound” are used interchangeably herein and refer to molecules, whether naturally-occurring or artificially created (e.g., via chemical synthesis) that have a relatively low molecular weight. Typically, an organic compound contains carbon. An organic compound may contain multiple carbon-carbon bonds, stereocenters, and other functional groups (e.g., amines, hydroxyl, carbonyls, or heterocyclic rings). In some embodiments, organic compounds are monomeric and have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body. In certain embodiments, the organic molecule is known to bind and/or cleave a nucleic acid. In some embodiments, the organic compound is an enediyne. In some embodiments, the organic compound is an antibiotic drug, for example, an anticancer antibiotic such as dynemicin, neocarzinostatin, calicheamicin, esperamicin, bleomycin, or a derivative thereof.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode.

### DETAILED DESCRIPTION

Some aspects of this disclosure describe the development of probes that can react with nucleophilic nucleic acids to form nucleic acid-probe conjugates. Such probes are referred to herein as “electrophilic activity-based probes” or “activity-based probes.” For example, some aspects of this disclosure provide probes that react with a nucleophilic nucleotide residue, for example, a nucleophilic G nucleotide in the context of a stem-bulge-stem-loop structure of a reactive nucleic acid. In some embodiments, the probes provided herein are small molecules that typically include a reactive moiety capable of reacting with a nucleophilic G nucleotide, e.g., a disubstituted epoxide moiety, and also a functional group, e.g., a detectable label or reactive handle, such as a click chemistry handle, that allows labeling the probe and/or the probe-labeled nucleic acid, for example, for purposes of detection, quantification, isolation, and/or further chemical modification.

The probes provided herein are useful, for example, for labeling, identifying, detecting, quantifying, and/or isolating reactive nucleic acids. The probes may be used to label any nucleic acid comprising a nucleophilic residue including, but not limited to, naturally occurring RNAs or DNAs, engineered nucleic acids comprising a nucleophilic residue, and reactive nucleic acid tags. The probes provided herein are useful for labeling reactive nucleic acids, for example, reactive RNA tags in vitro or in vivo.

In addition, this disclosure provides consensus sequences and the minimal sequence requirements for an exemplary reactive nucleic acid secondary structure comprising a nucleophilic nucleotide residue in the context of a nucleic acid sequence forming a stem-bulge-stem-loop structure. In some embodiments, the minimal reactive nucleic acid sequence comprises a reactive G nucleotide within the bulge structure that exhibits unusually high nucleophilic reactivity. Engineered nucleic acids comprising such minimal reactive sequences are also provided herein. For example, in some embodiments, the minimal reactive sequence is of the structure 5′-W¹G²A³G*R⁵N₄₋₃₀N⁶A⁷G⁸G⁹C¹⁰[U/T]¹¹C¹²R¹³-3′ (SEQ ID NO: 1), wherein R represents A or G; W represents A or [U/T]; [U/T] represents U or T; and N represents any nucleotide.

Exemplary methods for using activity-based probes for labeling, identifying, detecting, quantifying, and/or isolating reactive nucleic acids, for example, genome-encoded reactive RNAs or fusion molecules comprising a reactive nucleic acid, are also described herein. In addition, this disclosure provides kits comprising reagents for carrying out the methods disclosed herein. In the context of identifying naturally occurring reactive nucleic acids, the activity-based probes and methods disclosed herein are advantageous over current identification approaches that rely on secondary structure prediction or sequence homology with known RNAs in that the strategies disclosed herein enable the unbiased identification of chemically reactive nucleic acids, for example, naturally occurring nucleophilic RNAs.

### Activity-Based Nucleic Acid Probes

Some aspects of this disclosure provide electrophilic probes that can react with nucleophilic nucleic acids, for example, with nucleophilic RNAs or DNAs. In some embodiments, the electrophilic probes provided comprise an epoxide group. In some embodiments, the epoxide group is of Formula (E):

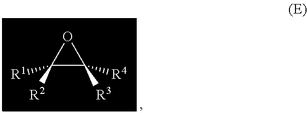

wherein R¹, R², R³, and R⁴, are, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{X}; —CO₂R^{X}; —CN; —SCN; —SR^{X}; —SOR^{X}; —SO₂R^{X}; —NO₂; —N(R^{X})₂; —NHC(O)R^{X}; or —C(R^{X})₃; wherein each occurrence of R^{X} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R¹, R², R³, and/or R⁴ is, independently, [CH]ₙ—R^{XX} or —[CH]ₙ—O—[CH]ₘR^{ZZ}; wherein n is an integer between 0 and 25, e.g., 0, 1, 2, 3, 4, 5, 6, 7, 8, 9, 10, 11, 12, 13, 14, 15, 16, 17, 18, 19, 20, 21, 22, 23, 24, or 25; and R^{XX} and/or R^{ZZ} is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{Y}; —CO₂R^{Y}; —CN; —SCN; —SR^{Y}; —SOR^{Y}; —SO₂R^{Y}; —NO₂; —N(R^{Y})₂; —NHC(O)R^{Y}; or —C(R^{Y})₃; wherein each occurrence of R^{Y} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, R^{XX} and/or R^{ZZ} is, independently, —O—(C═O)—[CH]ₙ—R^{Q}, wherein n is an integer between 0 and 20, e.g., 0, 1, 2, 3, 4, 5, 6, 7, 8, 9, 10, 11, 12, 13, 14, 15, 16, 17, 18, 19, 20, 21, 22, 23, 24, or 25; and R^{Q} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{R}; —CO₂R^{R}; —CN; —SCN; —SR^{R}; —SOR^{R}; —SO₂R^{R}; —NO₂; —N(R^{R})₂; —NHC(O)R^{R}; or —C(R^{R})₃; wherein each occurrence of R^{R} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, the activity-based electrophilic probe comprises a disubstituted epoxide. In some embodiments, the disubstituted epoxide is a 2,3-disubstituted epoxide. In some embodiments, the epoxide is a cis-epoxide, e.g., a cis-disubstituted epoxide. In some embodiments, the epoxide is a trans-epoxide.

In some embodiments, the activity-based electrophilic probe comprises or consists of a structure of Formula (F):

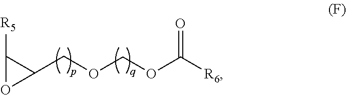

wherein p is an integer between 1 and 10, inclusive; q is an integer between 1 and 10, inclusive; R⁵ and/or R⁶ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{T}; —CO₂R^{T}; —CN; —SCN; —SR^{T}; —SOR^{T}; —SO₂R^{T}; —NO₂; —N(R^{T})₂; —NHC(O)R^{T}; or —C(R^{T})₃; wherein each occurrence of R^{T} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R⁵ and/or R⁶ is, independently, [CH]ₙ—R^{TT}; wherein n is an integer between 0 and 25, e.g., 0, 1, 2, 3, 4, 5, 6, 7, 8, 9, 10, 11, 12, 13, 14, 15, 16, 17, 18, 19, 20, 21, 22, 23, 24, or 25; and R^{TT} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{U}; —CO₂R^{U}; —CN; —SCN; —SR^{U}; —SOR^{U}; —SO₂R^{U}; —NO₂; —N(R^{U})₂; —NHC(O)R^{U}; or —C(R^{U})₃; wherein each occurrence of R^{U} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, R⁵ is CH₃. In some embodiments, p is 1, 2, 3, 4, 5, 6, 7, 8, 9, or 10. In some embodiments, q is 1, 2, 3, 4, 5, 6, 7, 8, 9, or 10. In some embodiments, p and/or q is, independently, an integer between 2 and 10, inclusive; between 2 and 5, inclusive; or between 2 and 4, inclusive. In some embodiments, p is 3 and q is an integer between 2 and 10, inclusive; between 2 and 5, inclusive; or between 2 and 4, inclusive. In some embodiments, q is 2 and p is an integer between 2 and 10, inclusive; between 2 and 5, inclusive; or between 2 and 4, inclusive. In some embodiments, p is 3 and q is 2.

In some embodiments, C5 and C6 are in cis.

In some embodiments, the activity-based electrophilic probe is of Formula (1)

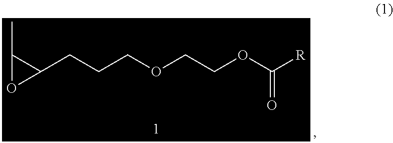

wherein R is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl, a protecting group, a reactive handle, such as a click chemistry handle, a detectable label; —OR^{A}; —C(═O)R^{A}; —CO₂R^{A}; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen, a protecting group, a reactive handle, such as a click chemistry handle, a detectable label, aliphatic, heteroaliphatic, acyl, aryl, heteroaryl, alkoxy, aryloxy, alkylthio, arylthio, amino, alkylamino, dialkylamino, heteroaryloxy, or heteroarylthio.

In some embodiments, R is —[CH]ₙ—R^{AA}; n is an integer between 0 and 25; and R^{AA} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl, a protecting group, a reactive handle, such as a click chemistry handle, a detectable label, —C(═O)R^{B}; —CO₂R^{B}; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen, a protecting group, a reactive handle, such as a click chemistry handle, a detectable label, aliphatic, heteroaliphatic, acyl, aryl, heteroaryl, alkoxy, aryloxy, alkylthio, arylthio, amino, alkylamino, dialkylamino, heteroaryloxy, or heteroarylthio. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, R comprises a detectable label. In some embodiments, the detectable label comprises a binding agent, a fluorescent or bioluminescent moiety, an enzyme, a ligand, a sequence tag, a radioactive isotope, or a mass tag. In some embodiments, the detectable label comprises biotin. In some embodiments, the detectable label comprises a fluorescent or bioluminescent moiety.

In some embodiments, the disubstituted epoxide is the compound of any one of Formula 9-16:

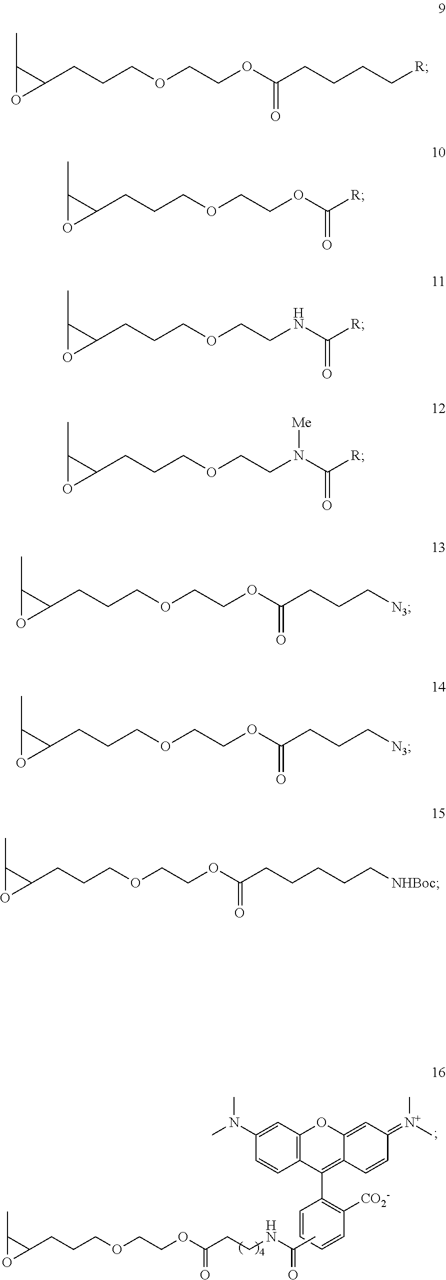

wherein R for Formula 9-12 is defined the same as for Formula 1 above.

### Self-Labeling Nucleic Acid Sequences

Some aspects of this disclosure provide reactive nucleic acid molecules that comprise a nucleophilic G nucleotide. Such reactive nucleic acid molecules are also referred to herein as “reactive nucleic acids,” “self-labeling nucleic acids,” “reactive tags” or “self-labeling tags,” because they can be conjugated to another molecule, for example, an electrophilic molecule comprising a detectable label without the requirement for additional reactants or catalysts but merely based on their nucleophilic reactivity. Such reactive nucleic acids thus provide a convenient approach for tagging and/or labeling any suitable molecule of interest.

For example, in some embodiments, a reactive nucleic acid as provided herein is used to tag a nucleic acid molecule of interest. In some embodiments, the nucleic acid molecule of interest is a transcript, for example, a messenger RNA. A messenger RNA can be tagged by generating an expression construct encoding the messenger RNA fused to a reactive nucleic acid, e.g., a nucleic acid comprising the consensus sequence provided in SEQ ID NO: 1. Such fusion constructs can be generated by recombinant technologies known to those of skill in the art, or by synthesizing an encoding sequence de novo. The fusion construct can be expressed, e.g., in a cell or tissue of interest. Suitable methods for expressing recombinant or synthetic constructs are well known to those of skill in the art. For example, transfection, transduction, or gene targeting methods may be used to contact the target cell or tissue with the fusion construct and express the fusion molecule in the target cell or tissue. Once the fusion molecule is expressed, the cell or tissue can be contacted with a suitable electrophilic probe, e.g., a probe provided herein. This contacting can be performed in vitro or in vivo. In some embodiments, the electrophilic probe comprises a disubstituted epoxide moiety as provided herein and a detectable label, e.g., a fluorescent moiety. Typically, the electrophilic probe will react with the nucleophilic G nucleotide of the reactive nucleic acid fused to the molecule of interest and thus covalently attach the detectable label to the molecule of interest. The presence, quantity, and/or location of the molecule of interest can then be determined by detecting the detectable label, e.g., by fluorescent microscopy or any other suitable method.

One advantage of the reactive nucleic acids provided herein is thus that molecules of interest can be covalently tagged and/or labeled in vitro or in vivo without a requirement to fix cells or tissues and based on covalent epoxide chemistry rather than non-covalent technologies, such as nucleic acid hybridization or protein-protein interactions. Further, the size and chemical nature of the minimal reactive nucleic acid sequence and of the electrophilic moiety of suitable probes allow tagging and/or labeling of molecules of interest in applications in which conventional binding agents, such as antibodies or hybridizing nucleic acid probes, would not be suitable.

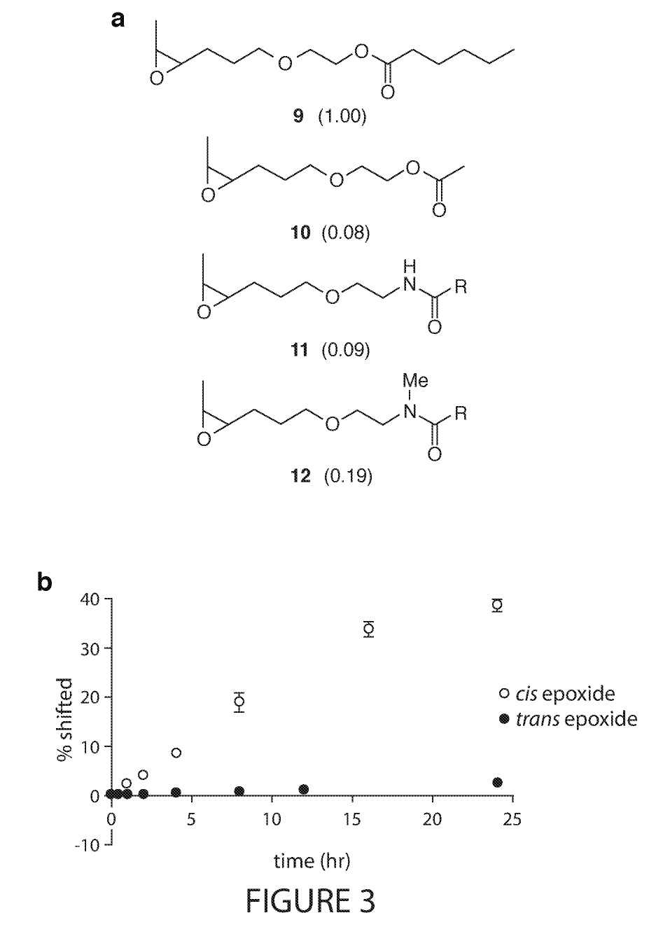

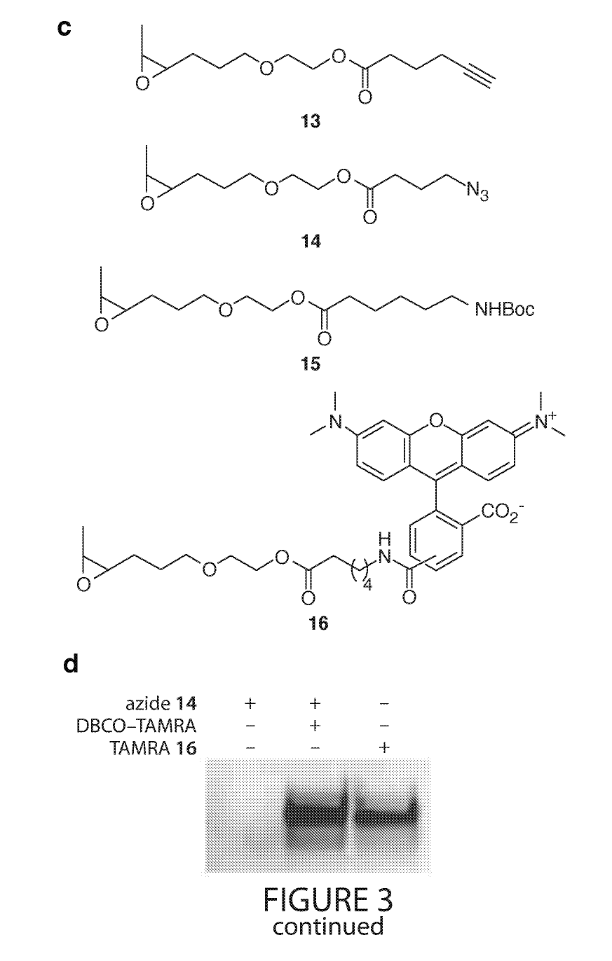

Accordingly, self-labeling nucleic acids as provided herein can be conjugated to a molecule of interest (e.g., to a nucleic acid, such as, for example, a coding or non-coding RNA or DNA, or to a protein, metabolite, or small molecule) and are useful for the detection, quantification, tracking, and/or isolation of such tagged molecules of interest in vitro or in vivo. The reactive nucleic acid sequences provided herein are thus useful as self-labeling tags that can be conjugated to molecules of interest to enable detecting, quantifying, and/or tracking the tagged molecules of interest. Methods of using such reactive, self-labeling nucleic acids are also provided herein, including, but not limited to, methods for using the reactive nucleic acids as self-labeling tags that can be conjugated to a molecule of interest. A non-limiting, exemplary embodiment, in which an RNA of interest is fused to a self-labeling nucleic acid and then covalently bound by an electrophilic probe conjugated to a detectable label is illustrated in FIG. 3 a.

Some aspects of this disclosure provide the minimal nucleic acid sequence required for nucleophilic reactivity of a self-labeling nucleic acid. The identification, analysis, and optimization of reactive nucleic acid sequences comprising a stem-bulge-stem-loop structure with a nucleophilic G nucleotide within the bulge are described in more detail elsewhere herein. As one of the results of the sequence analysis and optimization experiments disclosed herein, the minimal sequence requirements for a nucleic acid to be reactive with electrophilic activity-based probes were delineated. Accordingly, some aspects of this disclosure provide optimized consensus sequences of minimal nucleic acids that are reactive with the electrophilic activity-based probes provided herein.

One exemplary minimal, optimized consensus sequence that may be part of a reactive nucleic acid comprising a nucleophilic G nucleotide (G*) is:

(SEQ ID NO: 1)

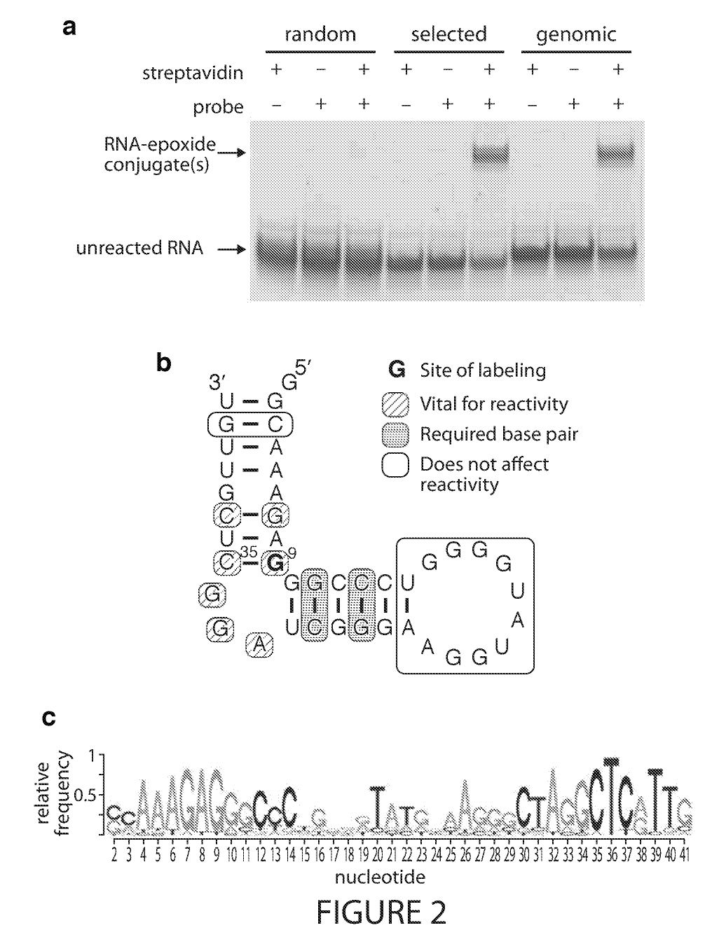

5′-W¹G²A³G*R⁵N₄₋₃₀N⁶A⁷G⁸G⁹C¹⁰ [U/T]¹¹C¹²R¹³-3′,

wherein R represents A or G; W represents A or [U/T]; [U/T] represents U or T; and N represents any nucleotide. In some embodiments, a nucleic acid comprising the consensus sequence of SEQ ID NO: 1 forms a stem-bulge structure in which the reactive G nucleotide (G*) is included. The nucleotide residues are numbered for ease of reference, except for the variable-length sequence of N₄₋₃₀. A non-limiting, exemplary embodiment of a reactive nucleic acid comprising a minimal reactive sequence and forming a stem-bulge structure is illustrated in FIG. 2b.

In some embodiments, the nucleotide sequence of SEQ ID NO: 1 comprises ribonucleotides. In some such embodiments, [U/T] represents U. In some embodiments, the nucleotide sequence of SEQ ID NO: 1 comprises deoxyribonucleotides. In some such embodiments, [U/T] represents T. In some embodiments, the nucleic acid comprising the consensus sequence of SEQ ID NO: 1 is an RNA molecule. In some embodiments, the nucleic acid comprising the consensus sequence of SEQ ID NO: 1 is a DNA molecule. In some embodiments, the nucleic acid comprising the consensus sequence of SEQ ID NO: 1 comprises both ribonucleotides and deoxyribonucleotides. In some embodiments, the nucleic acid comprising the consensus sequence of SEQ ID NO: 1 comprises non-naturally occurring nucleotides.

In some embodiments, the reactive G nucleotide is paired with a C nucleotide, for example, a C nucleotide of the same nucleotide strand. In some embodiments, the nucleotide immediately 5′ from the reactive G nucleotide is paired. In some embodiments, the nucleotide immediately 3′ from the reactive G nucleotide is paired. In some embodiments, the nucleotide immediately 5′ and the nucleotide immediately 3′ of the reactive G nucleotide are paired. In some embodiments, the nucleotide immediately 5′ from the C nucleotide that is paired with the reactive G nucleotide is unpaired and forms part of the bulge of the stem-bulge structure.

In some embodiments, the bulge comprises 1, 2, 3, 4, 5, 6, 7, 8, 9, or 10 unpaired nucleotides. In some embodiments, the bulge comprises three unpaired nucleotides. In some embodiments, the bulge comprises a 5′-AGG-3′ sequence. In some embodiments, the reactive G nucleotide pairs with a C nucleotide immediately 3′ of the bulge, and the nucleotide immediately 3′ of the reactive G nucleotide pairs with the nucleotide immediately 5′ of the bulge. In some embodiments, the reactive G nucleotide is comprised in the stem of the stem-bulge structure. In some embodiments, the nucleotide immediately 3′ of the reactive G nucleotide is comprised in the stem of the stem-loop structure. A non-limiting, exemplary embodiment of a reactive nucleic acid comprising a minimal reactive sequence and forming a stem-bulge-stem-loop structure is illustrated in FIG. 2b.

In some embodiments, all of the nucleotides in the 5′-W¹G²A³G*-3′ sequence within the consensus sequence are paired, forming the stem or part of the stem of the stem-bulge structure. In some embodiments, all of the nucleotides in the 5′-C¹⁰[U/T]¹¹C¹²R¹³-3′ sequence within the consensus sequence are paired, forming the stem or part of the stem of the stem-bulge structure. In some embodiments, the nucleotides in the 5′-W¹G²A³G*-3′ sequence pair with the nucleotides in the 5′-C¹⁰[U/T]¹¹C¹²R¹³-3′ sequence of the consensus sequence, forming the stem or part of the stem of the stem-bulge sequence.

In some embodiments, N₄₋₃₀ comprises the stem-loop structure of the nucleic acid. In some embodiments, N₄₋₃₀ comprises 4-25, 4-20, 4-15, 4-10, 6-30, 10-30, 15-30, or 20-30 nucleotides, for example, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the stem of the stem-loop structure comprises at least four paired nucleotides. In some embodiments, the stem of the stem-loop structure comprises 4, 5, 6, 7, 8, 9, 10, 11, 12, 13, 14, or 15 paired nucleotides. In some embodiments, N₄₋₃₀ comprises an even number of paired nucleotides, for example, 4, 6, 8, 10, 12, 14, 16, 18, or 20 paired nucleotides. In some embodiments, N₄₋₃₀ comprises an even number of paired nucleotides, for example, 5, 7, 9, 11, 13, or 15 paired nucleotides. Non-limiting examples of suitable sequences that can form a stem-loop structure are provided herein, e.g., in the Examples section and in FIG. 2b. Additional suitable sequences for N4-30 that can form a stem-loop structure in the context of a reactive nucleic acid as provided herein will be apparent to those of skill in the art based on this disclosure.

In some embodiments, the stem of the stem-loop structure comprises at least two G-C or C-G nucleotide pairs. In some embodiments, the nucleotide at the 5′-end of N₄₋₃₀ is a G or a C and forms a G-C or a C-G base pair with the nucleotide at the 3′-end of N₄₋₃₀. In some embodiments, the third nucleotide from the 5′-end of N₄₋₃₀ is a G or a C and forms a G-C or a C-G base pair with the third nucleotide from the 3′-end of N₄₋₃₀. In some embodiments, the stem of the stem-loop structure comprises G-C or C-G nucleotide pair at the second and/or the fourth nucleotide 3′ of the reactive G nucleotide. In some embodiments, R⁵ and N⁶ of SEQ ID NO: 1 are paired, forming the end of the stem most distal from the loop in the stem-loop structure. In some embodiments, the paired R⁵ and N⁶ nucleotides of SEQ ID NO: 1 form the end of the stem in the stem-loop structure that is immediately adjacent to the bulge in the stem-bulge-stem-loop structure formed by the nucleic acid comprising the nucleotide sequence of SEQ ID NO: 1.

In some embodiments, N₄₋₃₀ comprises a 5′-GCCC[U/T]N₁₀AGGGC-3′ sequence (SEQ ID NO: 2). In some embodiments, the stem-loop structure of the nucleic acid comprises the sequence 5′-R⁵GCCC[U/T]N₂₋₂₄AGGGCN⁶-3′ (SEQ ID NO: 20), wherein the 5′-R⁵GCCC[U/T]-3′ sequence (SEQ ID NO: 21) base-pairs with the 5′-AGGGCN⁶-3′ sequence (SEQ ID NO: 22), and wherein N₂₋₂₄ comprises a sequence of 2-24 unpaired nucleotides forming the loop of the stem-loop structure. In some embodiments, the loop of the stem-loop structure comprises 2, 3, 4, 5, 6, 7, 8, 9, 10, 11, 12, 13, 14, 15, 16, 17, 18, 19, 20, 21, 22, 23, or 24 unpaired nucleotides.

In some embodiments, the reactive nucleic acid comprises a 5′-WGAG*GGCCC[U/T]N₁₋₃G[U/T]A[U/T]GRAAGGGC[U/T]AGGC[U/T]C-3′ sequence (SEQ ID NO: 3). In some embodiments, the reactive nucleic acid comprises a 5′-GGCAAAGAG*GGCCC[U/T]N₁₋₃G[U/T]A[U/T]GRAAGGGC[U/T]AGGC[U/T]CR[U/T][U/T]G[U/T]-3′ sequence (SEQ ID NO: 4). In some embodiments, the reactive nucleic acid comprises a 5′-GGCAAAGAG*GGCCC[U/T]GGGG[U/T]A[U/T]GGAAGGGC[U/T]AGGC[U/T]CG[U/T][U/T]G[U/T]-3′ sequence (SEQ ID NO: 5). In some embodiments, the reactive nucleic acid comprises a 5′-GGCAAAGAG*GGCCC[U/T]AG[U/T]A[U/T]GAAAGGGC[U/T]AGG C[U/T]CA[U/T][U/T]G[U/T]-3′ sequence (SEQ ID NO: 6). In some embodiments, the reactive nucleic acid comprises a 5′-GGCCGCUCCAGAAGAGGGCCCCUUCGGGGGCUAGGCUCGAUGUCGGCC-3′ sequence (SEQ ID NO: 7).

An exemplary stem-bulge-stem-loop structure consistent with the consensus sequence of SEQ ID NO: 1 is shown in FIG. 3B. The reactive G nucleotide is labeled “G⁹” in the figure, and the base pairs required for nucleophilic reactivity of the reactive G nucleotide as well as the nucleotides and base pairs vital for reactivity are described. It will be apparent to those of skill in the art that the exemplary reactive (or self-labeling) nucleic acids described in detail herein, for example, the exemplary structures described above or the sequence provided in FIG. 3B, do not limit the scope of this disclosure. Additional suitable nucleic acids comprising a structure that conforms to the consensus sequence of SEQ ID NO: 1 and thus comprise a nucleophilic G nucleotide will be apparent to those of skill in the art based on the instant disclosure. The disclosure is not limited in this respect.

### Self-Labeling Fusion Molecules

Some aspects of this disclosure provide fusion molecules comprising a reactive nucleic acid provided herein, for example, a self-labeling nucleic acid comprising the nucleotide sequence of SEQ ID NO: 1, and a molecule of interest (e.g., a heterologous nucleic acid, a protein, a polysaccharide, a lipid, a lipoprotein, a metabolite, or a small molecule) that is conjugated to the reactive nucleic acid. In such fusion molecules, the reactive nucleic acid may function as a self-labeling tag that allows the detection, quantification, tracking, and/or isolation of the molecule of interest. Such fusion molecules can be conjugated to an electrophilic probe, for example, an electrophilic probe as provided herein. Depending on the structure of the electrophilic probe used, the fusion molecule can thus be labeled, or otherwise chemically modified, upon reaction of the nucleophilic self-labeling tag with the electrophilic probe.

Since the self-labeling tags provided herein comprise reactive nucleic acid sequences, any molecule of interest that can be conjugated to a nucleic acid can be tagged with such self-labeling nucleic acids. This allows for a wide variety of molecules of interest to be suitable for tagging with the reactive nucleic acids provided herein. The resulting fusion molecules comprising the molecule of interest and the reactive self-labeling nucleic acid can subsequently be contacted with an electrophilic probe to chemically modify the fusion molecule, e.g., by adding a detectable label or a reactive handle, such as a click chemistry handle.

In some embodiments, the molecule of interest in a fusion molecule provided herein is heterologous to the reactive nucleic acid in that the molecule of interest does not occur in nature conjugated to the reactive nucleic acid. For example, if the molecule of interest is a nucleic acid, it is heterologous to the self-labeling nucleic acid provided herein, if the naturally occurring forms of the nucleic acid of interest do not comprise the respective self-labeling nucleic acid. The fusion molecules provided in such embodiments, thus, exclude any naturally occurring molecules that comprise the self-labeling tags described herein. For example, the full-length A. Pernix catalytic RNA discovered as described elsewhere herein would not be included in the scope of the term “fusion molecule” as used in such embodiments because the naturally occurring A. Pernix RNA catalytic RNA comprises a minimal sequence constituting a self-labeling nucleic acid that conforms to SEQ ID NO: 1 in addition to other nucleotide sequences. If, on the other hand, a molecule of interest does not naturally occur in a form that comprises or is conjugated to a self-labeling tag provided herein, for example, a self-labeling tag comprising a nucleotide sequence of SEQ ID NO: 1, it would qualify as “heterologous” to the self-labeling tag.

Suitable molecules of interest that can be conjugated to reactive nucleic acid sequences provided herein include, but are not limited to, heterologous nucleic acids, proteins, polysaccharides, lipids, lipoproteins, metabolites, and small molecules. Suitable nucleic acids include, without limitation, RNAs, DNAs, RNA/DNA hybrids, and nucleic acids comprising non-naturally occurring nucleotides or other chemical modifications. In some embodiments, a nucleic acid of interest may comprise a genomic sequence, a sequence encoding a gene product (e.g., a transcript or protein), or a non-coding sequence. In some embodiments, a nucleic acid of interest may be a primer or a probe, for example, a hybridization probe. In some embodiments, a nucleic acid of interest may be a coding RNA, such as an mRNA or a primary transcript. In some embodiments, a nucleic acid of interest may be a non-coding RNA, such as, for example, a transfer RNA (tRNA), a ribosomal RNA (rRNA), a snoRNA, microRNA, siRNA, snRNA, shRNA, exRNA, piRNA, or a lncRNAs (e.g., Xist or HOTAIR). In some embodiments, a nucleic acid of interest may be a binding agent, for example, and aptamer. Additional suitable heterologous nucleic acids will be apparent to those of skill in the art based on the instant disclosure as the invention is not limited in this respect.

In some embodiments, the molecule of interest is a protein. In some embodiments, the protein is a binding protein, such as, for example, an antibody, an antigen-binding antibody fragment, a ligand, or a receptor. In some embodiments, the protein is an enzyme, for example, an oxidoreductase, a transferase, a hydrolase, a lyase, an isomerase, a kinase, a phosphatase, or a ligase. In some embodiments, the protein is a structural protein. In some embodiments, the protein is involved in cell signaling and/or ligand binding. In some embodiments, the protein is a cellular protein. In some embodiments, the protein is an excreted protein. In some embodiments, the protein is a transmembrane protein. In some embodiments, the protein is a cell surface protein. In some embodiments, the protein binds to a cellular protein, for example, a cell surface protein that is specifically expressed by cells in a diseased or pathological state. In some embodiments, the protein is a therapeutic or diagnostic protein. Additional suitable proteins will be apparent to those of skill in the art based on the instant disclosure as the invention is not limited in this respect.

In some embodiments, the molecule of interest is a metabolite. In some embodiments, the metabolite is an intermediate or a product of a cell's or a subject's metabolism. In some embodiments, the metabolite is a small molecule. In some embodiments, the metabolite plays a role in cellular or inter-cellular signaling, stimulates or inhibits an enzyme, or exhibits catalytic activity (e.g., as a cofactor of an enzyme).

In some embodiments, the molecule of interest is a small molecule. For example, in some embodiments, the molecule of interest is a therapeutic small molecule, for example, a small molecule drug. In other embodiments, the molecule of interest is a diagnostic small molecule, for example, a small molecule that binds to a biomarker, such as, for example, a cell surface protein that is indicative of a diseased or pathological state.

In some embodiments, the conjugation of a self-labeling tag as provided herein to a molecule of interest does not interfere with the structure or the function exhibited by the molecule of interest in its untagged form. For example, in some embodiments, the tagged molecule of interest retains at least 50%, 60%, 70%, 80%, 90%, 95%, 96%, 97%, 98%, or 99% of its original function. For example, if the molecule of interest is an enzyme, the tagged enzyme will catalyze its original reaction at an efficiency of at least 50%, 60%, 70%, 80%, 90%, 95%, 96%, 97%, 98%, or 99% of its original reaction rate under the same conditions. For another example, if the molecule of interest is a binding agent, the tagged binding agent will bind to its original binding partner(s) with an affinity of at least 50%, 60%, 70%, 80%, 90%, 95%, 96%, 97%, 98%, or 99% of its original affinity. For yet another example, if the molecule of interest is a small molecule drug, the tagged drug will exhibit at least 50%, 60%, 70%, 80%, 90%, 95%, 96%, 97%, 98%, or 99% of its original therapeutic activity. Suitable methods and approaches for measuring and comparing the activity of untagged and tagged molecules of interest are well known to those of skill in the art. Such methods include, without limitation, enzymatic assays and binding assays as well as therapeutic assays in animal models or in a clinical setting. It will be understood by those of skill in the art that the use of such assays to confirm that the molecule of interest retains some or all of its original function constitutes routine experimentation.

In some embodiments, the fusion molecule comprises multiple copies of the reactive self-labeling tag. In some embodiments, using multiple copies of the reactive self-labeling tag may increase the efficiency of labeling the fusion molecule with an electrophilic probe. In some embodiments, using multiple copies of the reactive self-labeling tag may increase the sensitivity of a detection assay for detecting the fusion molecule, since a single fusion molecule may be conjugated to multiple probes comprising a detectable label.

In some embodiments, the fusion molecule comprises 2-10 copies of a reactive nucleic acid. For example, in some embodiments, a fusion molecule as provided herein may comprise 2, 3, 4, 5, 6, 7, 8, 9, or 10 copies of a self-labeling nucleic acids tag as provided herein. In some embodiments, the fusion molecule comprises three copies of a reactive nucleic acid. It will be understood by those of skill in the art that in embodiments of fusion molecules comprising multiple self-labeling tags, the tags may be of the same nucleic acid sequence or comprise different nucleic acid sequences that conform to a reactive nucleic acid sequence provided herein, for example, to the consensus sequence of SEQ ID NO: 1.

In some embodiments, a self-labeling nucleic acid as provided herein is conjugated to a molecule of interest via a covalent bond. The bond between the self-labeling nucleic acid and the molecule of interest will depend on the nature of the molecule of interest. For example, if the molecule of interest is a heterologous nucleic acid, the self-labeling nucleic acid can be fused to the nucleic acid of interest via a phosphodiester bond. Such fusion molecules may be created, for example, via recombinant technologies well known to those of skill in the art. In other embodiments, where the molecule of interest is not a nucleic acid, the self-labeling nucleic acid can be conjugated to the molecule of interest by a known chemical method. Suitable chemical methods will be apparent to those of skill in the art and include, without limitation, the use of click chemistry functional groups to functionalize and conjugate a self-labeling nucleic acid as provided herein to a heterologous molecule of interest. The self-labeling nucleic acid may be conjugated to the molecule of interest, for example, and without limitation, via a C—C bond, a C—O bond, a C—(C═O) bond, a C—N bond, or an S—S bond.

In some embodiments, the reactive nucleic acid provided herein is conjugated to the molecule of interest directly, i.e., without a molecule or structure separating the tag from the molecule of interest. In other embodiments, however, the reactive nucleic acid is conjugated to the molecule of interest via a linker. The use of a linker may be desirable, for example, in order to provide a specific spacing between the tag and the molecule of interest, to confer specific structural, biological, or chemical properties to the fusion molecule, or to allow for the tag to be separated from the molecule of interest, for example, by using a cleavable linker. In some embodiments, a cleavable linker may comprise a target site for an enzyme, for example, for a nuclease or a protease. In other embodiments, a cleavable linker may comprise a chemical structure that is a substrate for a cleaving enzyme, for example, a polysaccharide structure. In some embodiments, a cleavable linker may be a photocleavable linker, for example, a linker that can be cleaved by exposure to UV light.

In some embodiments, a linker may comprise an organic polymer structure, for example, a polysaccharide structure, a polynucleotide structure, or a polypeptide structure. In some embodiments, a suitable linker may be comprised of the same type of molecule as the nucleic acid or the molecule of interest. For example, a suitable linker may be a nucleic acid. In some embodiments, where the molecule of interest is a protein, a suitable linker may be a polypeptide sequence. In some embodiments, a linker may be a hybrid molecule, for example, comprising a nucleic acid sequence and a polypeptide sequence. In some embodiments, the linker may comprise a synthetic polymer, such as PEG.

In some embodiments, the molecule of interest is conjugated to the 5′-end of the reactive nucleic acid. In some embodiments, the molecule of interest is conjugated to the 3′-end of the reactive nucleic acid. In some embodiments, where multiple copies of the reactive nucleic acids tag are used, the molecule of interest may be flanked on both ends with one or more copies of a reactive nucleic acid.

In some embodiments, the molecule of interest is an RNA molecule, and the reactive probe is an RNA probe. In some embodiments, the fusion molecule comprises or is of the structure: 5′-[RNA of interest]ₙ-[optional linker RNA sequence]-[reactive RNA tag]-3′, wherein n is an integer between 1 and 12. In some embodiments, the fusion molecule comprises or is of the structure: 5′-[reactive RNA tag]-[optional linker RNA sequence]-[RNA of interest]ₙ-3′, wherein n is an integer between 1 and 12.

Some aspects of this disclosure provide methods and reagents for generating fusion molecules comprising a reactive nucleic acid sequence provided herein and a heterologous molecule of interest. Typically, such methods include the conjugation a reactive nucleic acid sequence provided herein to a molecule of interest. This may be achieved by any suitable method known in the art. For example, if the molecule of interest is a nucleic acid, a fusion nucleic acid comprising both the reactive nucleic acid and the nucleic acid of interest may be generated by recombinant technologies. In some embodiments, an expression construct may be synthesized that either constitutes the fusion nucleic acid or that encodes a transcript comprising the fusion molecule. Where the molecule of interest is not a nuclei acid, it can be conjugated to the reactive nucleic acid by methods well known to those of skill in the art.

### Methods for Labeling and Detecting Reactive Nucleic Acids

Some aspects of this disclosure provide methods for labeling reactive nucleic acids as provided herein. Such methods typically include contacting the reactive nucleic acid, which may be isolated or conjugated to a molecule of interest, with an electrophilic probe comprising a detectable label, such as a fluorescent or luminescent moiety, a binding agent, or a mass tag. The contacting is performed under conditions suitable for the electrophilic probe to form a covalent bond with the nucleophilic G nucleotide of the reactive nucleic acid. Such conditions include, in some embodiments, physiological conditions, e.g., a temperature between 20 and 37° C., a pH between 5.5 and 7.5, and an ionic strength of about 200-350 mOsm. In some embodiments, the contacting is performed in vitro, e.g., in a reaction vial or in a cell or tissue culture.

In some embodiments, the reactive nucleic acid is isolated in a cell-free system and the contacting is performed in such a system, for example, in the context of a laboratory assay. In some embodiments, a fusion molecule comprising a reactive nucleic acid is used that comprises a binding agent, for example, a nucleic acid sequence or a protein sequence that binds to a target molecule, for example, by base pair-mediated hybridization to a complementary sequence, or by an antigen-antibody or ligand-receptor interaction. In some such embodiments, the reactive nucleic acid of the bound molecule is contacted with an electrophilic probe comprising a detectable label. Such embodiments may be carried out, for example, in the context of a detection assay, such as a northern, southern, or western blot, or an ELISA, or a fluorescent or bioluminescent microtiter plate-based assay. Once hybridized, the fusion molecule may be contacted with an electrophilic probe comprising a disubstituted epoxide conjugated to a suitable detectable label, e.g., to a fluorescent or bioluminescent moiety, under conditions suitable for the probe to bind the reactive nucleic acid. The probe will typically bind to the reactive nucleic acid in a specific manner, in that it will not bind to non-reactive nucleic acid species or any other biomolecules in the sample or assay. The nucleic acid, and thus the hybridized target sequence may then be detected by an appropriate fluorescent or bioluminescent assay.

For example, a fusion molecule comprising a reactive nucleic acid and a nucleic acid of interest that comprises a sequence capable of hybridizing to a target nucleic acid sequence may be contacted with a sample comprising the target nucleic acid sequence, e.g., immobilized on a solid support such as, for example, a membrane, under conditions suitable for the nucleic acid of interest to specifically hybridize to the target sequence. Once hybridized, the fusion molecule may be contacted with an electrophilic probe comprising a disubstituted epoxide conjugated to a detectable label, for example, to a fluorescent or bioluminescent moiety, under conditions suitable for the probe to bind the reactive nucleic acid. The nucleic acid, and thus the hybridized target sequence may then be detected by an appropriate fluorescent or bioluminescent assay.

Those of skill in the art will understand that the detection methods described herein are not limited to in vitro assays in which a target molecule bound by the molecule of interest is bound via nucleic acid hybridization. Additional embodiments will be apparent to those of skill in the art, including, but not limited to, for example, embodiments in which cell surface antigens are labeled under conditions conducive to cell survival or in vivo methods, in which, for example, biomarker molecules are bound by the molecule of interest and then labeled by contacting a reactive nucleic acid conjugated to the molecule of interest with an electrophilic probe comprising a detectable label. Some non-limiting examples of such embodiments are provided below, and additional embodiments will be apparent to those of skill in the art based on this disclosure.

For example, in some embodiments, the reactive nucleic acid is inside a cell, e.g., expressed from an expression construct, either in isolated form or fused to a molecule of interest, e.g., an RNA of interest, by the cell. In some such embodiments, the contacting is performed under conditions suitable for the cells to survive. In other embodiments, the cells are fixed before being contacted with the electrophilic probe.

In some embodiments, the contacting of the reactive nucleic acid with the electrophilic probe is performed in vivo. For example, in some embodiments, the reactive nucleic acid or a fusion molecule comprising the reactive nucleic acid and a molecule of interest is administered to a subject and is contacted with an electrophilic probe in the subject. In other embodiments, the reactive nucleic acid or a fusion molecule comprising the reactive nucleic acid may be contacted with an electrophilic probe in vitro under conditions suitable for the probe to bind to the reactive nucleic acid, and the resulting labeled nucleic acid or labeled fusion molecule may subsequently be administered to a subject.

The methods provided herein are also useful for the detection of molecules of interest in vitro or in vivo. For example, a fusion molecule comprising a reactive nucleic acid and a nucleic acid of interest, e.g., an mRNA of interest, can be used to determine the presence, quantity, and/or localization of the nucleic acid of interest in a cell or a tissue in vitro or in vivo. Such a fusion molecule may be expressed in a target cell from an expression construct encoding the fusion molecule under the control of a heterologous promoter. The expression construct may be introduced into the target cell via any suitable method known in the art, for example, via transfection, transduction, electroporation, or viral infection methods. Alternatively, the fusion molecule may be introduced into a cell of interest directly, e.g., via microinjection, transfection, or transduction. Once the fusion molecule is expressed into or introduced into the cell, the cell can be contacted with an electrophilic probe as provided herein. The electrophilic probes provided herein will typically not react with any non-reactive nucleic acid, e.g., with any nucleic acid endogenously expressed by the cell that does not comprise a sequence that conforms with the consensus sequence of SEQ ID NO: 1, but will specifically react with the reactive G nucleotide of the reactive nucleic acid molecule. In some embodiments, the probe comprises a detectable label, for example, a fluorescent moiety, such as a FITC or TAMRA moiety. The labeled fusion molecules can thus be detected after the probe has been incubated for a time sufficient for the reactive nucleic acid and the probe to react. In some embodiments, any unreacted probe is washed away from the cell, e.g., by changing the culture media. Any bound probe can subsequently be detected by any suitable method, for example, by fluorescent microscopy, confocal microscopy, FACS, ELISA, etc. The type of assay used will, of course, depend on the nature of the detectable moiety. Exemplary suitable detectable moieties and detection methods are provided herein, and additional suitable detectable moieties and detection methods will be apparent to those of skill in the art based on this disclosure.

The technology provided herein can also be used in a therapeutic or a diagnostic context. For example, in some embodiments, the molecule of interest is not a nucleic acid, but, for example, a protein, a polysaccharide, a lipid, a lipoprotein, a metabolite, or a small molecule. In such embodiments, the fusion molecule comprising the molecule of interest and the reactive nucleic acid are generated by synthetic methods instead of by expressing a construct encoding the fusion molecule. For example, in some embodiments, the fusion molecule may comprise a binding agent, such as, for example, an antibody or an antibody fragment, that specifically binds a cell type associated with a disease or disorder, for example, a neoplastic cell type. In some embodiments, a fusion molecule is synthesized that comprises the antibody or antibody fragment and a reactive nucleic acid, for example, a reactive RNA confirming to the minimal consensus sequence of SEQ ID NO: 1. In some embodiments, the fusion molecule is administered to a subject known to have a disease or disorder associated with the cell type bound by the antibody or antibody fragment, for example, to a subject having a tumor the cells of which express a specific tumor antigen. In some embodiments, the fusion molecule is administered in an amount effective to saturate all antigen sites of the tumor antigen within the subject, thus labeling the tumor cells with reactive nucleic acid molecules.

In some embodiments, an electrophilic probe as provided herein that is conjugated to a diagnostic agent, such as a detectable label, is administered to the subject after any unbound fusion molecule is cleared from the subject's body, e.g., via renal elimination. In some embodiments, the probe conjugated to the chemotherapeutic agent binds the fusion molecule that is bound to the tumor cells, thus creating a high concentration of detectable label in the vicinity of the tumor. In some embodiments, the fusion molecule comprises a plurality of reactive nucleic acid sequences, so that a single binding agent, e.g., an antibody or antibody fragment, can be labeled with a plurality of detectable labels. In some embodiments, the tumor cells can be detected or identified, or the boundary of the tumor and healthy tissue be determined.

In some embodiments, an electrophilic probe as provided herein that is conjugated to a chemotherapeutic agent, such as a platinum agent, is administered to the subject after any unbound fusion molecule is cleared from the subject's body, e.g., via renal elimination. In some embodiments, the probe conjugated to the chemotherapeutic agent binds the fusion molecule that is bound to the tumor cells, thus creating a high concentration of chemotherapeutic agent in the vicinity of the tumor. In some embodiments, the fusion molecule comprises a plurality of reactive nucleic acid sequences, so that a single binding agent, e.g., an antibody or antibody fragment, can be labeled with a plurality of chemotherapeutic agents. In some embodiments, the probe is conjugated to the chemotherapeutic agent via cleavable linker, for example, a polypeptide linker comprising a protease cleavage site that is susceptible to a protease specifically secreted by the tumor cells, such as a matrix metalloprotease. In some embodiments, the linker of tumor-bound probe is cleaved by the tumor-specific metalloprotease, thus releasing the chemotherapeutic agent from the probe at the site of the tumor.

Methods for identifying, detecting, quantifying, and/or tracking molecules of interest in vivo or in vitro as provided herein typically comprise providing a fusion molecule comprising a molecule of interest and a self-labeling nucleic acid conjugated to the molecule of interest, wherein the reactive nucleic acid comprises a sequence that conforms to the consensus sequence of SEQ ID NO: 1; contacting the fusion molecule with an electrophilic probe, e.g., a probe comprising a disubstituted epoxide, that comprises a detectable label under conditions suitable for the probe to form a covalent bond with the reactive G nucleotide (G*) within the consensus sequence; and detecting the detectable label bound to the molecule of interest.

Such method are useful, for example, for quantifying a molecule of interest in a biological sample. For example, if the molecule of interest is a transcript, the methods provided herein allow for convenient labeling and detection of the transcript by a standard chemical reaction, in which the detectable label can be interchanged to suit the specific requirements of the respective detection assay. The methods provided herein also allow, in some embodiments, to rely on the same bond-forming chemistry, e.g., based on disubstituted epoxide probes, for labeling different types of molecules of interest with different detectable labels, and thus provide a versatile platform for conducting various assays based on the same general labeling technology.

Suitable in vitro assays in which fusion molecules as provided herein can be used to detect, quantify, and/or track a target molecule will be apparent to those of skill in the art based on the instant disclosure. Such assays include, without limitation, hybridization-based assays, such as northern and southern blot assays, as well as FISH assays; binding interaction-based assays, such as western blotting assay, enzyme-linked immunosorbent assays (ELISA), an enzyme-linked immunospot assay (ELISPOT), lateral flow test assays, enzyme immunoassays (EIA), fluorescent polarization immunoassays (FPIA), chemiluminescent immunoassays (CLIA), antibody sandwich capture assays, isoelectric focusing assays, FACS assays, MACS assays; as well as assays for the detection of mass tags, such as tandem mass spectrometry assays.

In some embodiments, methods for detecting a fusion molecule comprising a molecule of interest and a reactive nucleic acid as provided herein comprise contacting a cell or tissue with the fusion molecule and subsequently detecting, quantifying, tracking, and/or isolating the fusion molecule, and in some embodiments, any target molecule or cell bound to the molecule of interest. In some embodiments, the contacting and/or the detecting, quantifying, tracking, and/or isolating the fusion molecule is in vitro. In some embodiments, the contacting and/or detecting, quantifying, tracking, and/or isolating is in vivo. For example, in some embodiments, the fusion molecule may be contacted with a probe comprising a detectable label that can be detected in vivo, such as a detectable moiety that can be detected via non-invasive imaging technologies. Suitable non-invasive assays and the respective detectable moieties that can be used for such assays will be apparent to those of skill in the art based on the instant disclosure. Suitable assays include, without limitation radiography assays, magnetic resonance imaging (MRI), ultrasound, photoacoustic imaging, thermography, tomography, and functional near-infrared spectroscopy.

In some embodiments where a fusion molecule as provided herein is labeled and/or detected in vivo, the fusion molecule and/or the electrophilic probe comprising a detectable label are administered to a subject. In some embodiments, the fusion molecule and/or the detectable label are administered in the form of a pharmaceutical composition. In some embodiments, the pharmaceutical composition is essentially pyrogen-free. In some embodiments, the pharmaceutical composition comprises an effective amount of the fusion molecule and/or of the electrophilic probe, and a pharmaceutically acceptable carrier or excipient. The effective amount of a fusion molecule provided herein will depend on the nature of the molecule of interest that is conjugated to the reactive, self-labeling nucleic acid. If the molecule of interest is a binding agent that can bind a target molecule, such as an antibody or an antibody fragment, an aptamer, or a nucleic acid sequence that can hybridize to a target nucleic acid, the effective amount is, in some embodiments, an amount that effects detectable binding of the fusion molecule to the target molecule. Similarly, an effective amount of an electrophilic probe as provided herein is an amount that effects detectable bond-formation of the probe with a reactive nucleic acid as provided herein.

Some aspects of this disclosure provide methods for identifying and detecting reactive nucleic acids, such as, for example, naturally occurring reactive cellular RNAs or DNAs. Such methods comprise contacting a biological sample comprising a naturally occurring reactive RNA or DNA with an electrophilic probe as provided herein, for example, a disubstituted epoxide probe comprising a detectable label, under conditions suitable for the electrophilic probe to form a covalent bond with the reactive RNA or DNA. In some embodiments, the biological sample comprises a cell, a tissue, a cell extract, a tissue extract, or a body fluid, such as blood, serum, plasma, urine, saliva, sweat, or tears. In some embodiments, the biological sample comprises an environmental sample, e.g., a sample comprising soil, water, or organic matter obtained from the environment. In some embodiments, the biological sample comprises a library of candidate RNA or DNA molecules, e.g., a library extracted from a cell or tissue, or a library derived from such a source with some intervening steps, such as reverse transcription, expression cloning, and re-expression in vitro. In some embodiments, the library is normalized. In some embodiments, the method of identifying a reactive RNA or DNA further comprises detecting any RNA or DNA molecule(s) bound to the probe. Typically, this step includes some form of isolation of the bound RNA molecule(s) from the unbound molecules in the biological sample. This may be achieved by any suitable isolation assay, including, but not limited to, binding assays, such as, for example, membrane- or bead-based binding assays. In some embodiments, the electrophilic probe comprises a binding agent, such as an antibody, or antibody fragment, or biotin. Any RNA or DNA molecule bound to the probe can be separated from the unbound molecules in the sample based on being conjugated to the binding agent via the probe. Some exemplary assays that are suitable for this separation and subsequent sequence determination are described in more detail elsewhere herein. Additional suitable assays will be apparent to those of skill in the art based on the instant disclosure and the invention is not limited in this respect.

### Kits

Some aspects of this disclosure provide kits for detecting, quantifying, and/or tracking a molecule of interest. In some embodiments, the kit comprises a nucleic acid comprising or encoding a self-labeling nucleic acid comprising a reactive G nucleotide (G*) in the context of a 5′-WGAG*RN₄₋₃₀AGGC[U/T]CR-3′ (SEQ ID NO: 1) nucleotide sequence, wherein R represents A or G; W represents A or [U/T]; [U/T] represents U or T; and N represents any nucleotide. The kit further comprises reagents for conjugating the self-labeling nucleic acid to a molecule of interest. In some embodiments where the kit is suitable for conjugating a nucleic acid of interest to the reactive self-labeling nucleic acid, the kit comprises a cloning or expression construct for cloning and/or expressing a recombinant fusion molecule comprising the nucleic acid of interest and the reactive self-labeling nucleic acid. In some embodiments, the kit further comprises an electrophilic probe comprising a detectable label for labeling the reactive G nucleotide (G*) of the reactive self-labeling nucleic acid. In some embodiments, the probe comprises a disubstituted epoxide conjugated to a detectable label. In some embodiments, the disubstituted epoxide is a 2,3-disubstituted epoxide. In some embodiments, the disubstituted epoxide comprises an ester moiety. In some embodiments, the epoxide is a cis-epoxide. In some embodiments, the detectable label comprises a binding agent, a fluorescent or bioluminescent moiety, a sequence tag, a radioactive isotope, a mass tag, or a reactive handle, such as a click chemistry handle. In some embodiments, the click chemistry handle is selected from the group consisting of terminal alkyne, azide, strained alkyne, diene, dieneophile, alkoxyamine, carbonyl, phosphine, hydrazide, thiol, tetrazine, and alkene.

In some embodiments, the kit comprises an expression construct comprising a nucleic acid sequence encoding the reactive self-labeling nucleic acid and a cloning site for inserting a heterologous nucleic acid sequence encoding a gene product of interest to generate a hybrid nucleic acid sequence encoding a fusion of the self-labeling nucleic acid and the gene product of interest. In some embodiments, the gene product of interest is an RNA. In some embodiments, the RNA is a non-coding RNA. In some embodiments, the gene product of interest is a binding agent. In some embodiments, the gene product of interest is an aptamer. In some embodiments, the encoded fusion is under the control of a heterologous promoter that can drive expression of the fusion transcript in eukaryotic or prokaryotic cells. In some embodiments, the kit further comprises reagents for delivering the expression construct to a target cell, for example, a transfection agent.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### Examples

### Materials and Methods

General.

Unless otherwise noted, all starting materials were obtained from commercial suppliers and were used without further purification.

LC/MS Screen of RNA Modification by Small-Molecule Probes.

A RNA pool of random sequence (N₈₀) (1 μg), 150 mM NaCl, and 50 mM Na-HEPES, pH 7.4, was incubated at 65° C. for 5 minutes, then cooled at room temperature for 5 minutes. MgCl₂ (10 mM) was then added to a total volume of 25 μL and the solution incubated for 10 minutes at room temperature, followed by addition of the small-molecule probe in DMSO (25 mM). After incubation at room temperature for 16 hours, the RNA was precipitated by addition of 5 μL of 3 M NaOAc and 180 μL of EtOH. Following EtOH precipitation, the RNA was taken up in 49 μL of 50 mM NH₄OAc, pH 4.5. Nuclease P1 (0.5 U, Wako Chemicals) was added, and the solution was incubated at 37° C. for 1 hour. Following lyophilization, the powder was resuspended in 45 μL H₂O.

RNA modifications were detected using negative-ion mode on a Waters Acquity ultra-performance liquid chromatography (UPLC) quadrupole TOF Premier mass spectrometer. LC was performed using a gradient from 0.1% (w/v) aqueous ammonium formate (A1) to methanol (B1) on an Acquity UPLC BEH C18 column (1.7 m, 2.1 mm×100 mm, Waters) at constant flow rate of 0.3 mL min⁻¹. The mobile phase composition was: 100% A1 for 3 min; linear increase over 8 min to 100% B1; maintain at 100% B1 for 2 min; return to 100% A1 over 1 min. Electrospray ionization used a capillary voltage of 3 kV, a sampling cone voltage of 40 V, and a low-mass resolution of 4.7. The desolvation gas temperature was 300° C., the flow rate was 800 L h⁻¹, and the source temperature was 150° C.

In Vitro Selection.

Fragmented genomic DNA pools were constructed as described previously.⁴⁴ DNA (0.2 μM) from the preceding round of the selection (or the starting pool for the first round) was incubated with 1×T7 RNA polymerase buffer (NEB), 1 mM of each rNTP, 5 mM DTT, and 28 μL of T7 RNA Polymerase (NEB) in 700 μL reaction volume for 10 hours at 37° C. The transcription mixture was divided into two halves and 30 μL 4 M NaCl and 1 mL of EtOH was added to each half. Following EtOH precipitation, the RNA was purified on a 10% TBE-UREA PAGE Criterion gel (Bio-Rad; 240 V for 35 minutes). The excised gel containing the desired RNA was incubated in 300 mM NaCl (450 μL) at 4° C. overnight, at which point it was precipitated with ethanol and resuspended in 125 μL H₂O.

The entire RNA pool was incubated at 37° C. for 15 minutes in 1× DNase buffer with 20 U DNase I (NEB). The solution was adjusted to 300 mM NaCl and 200 μL total volume and the RNA isolated by phenol/chloroform extraction, followed by EtOH precipitation. The resulting RNA pellet was resuspended in 100 μL H₂O.

The reaction between small-molecule probe(s) and the RNA pool was performed as follows: the RNA pool (1 μM) in buffer containing 150 mM NaCl, 25 mM Na-HEPES, pH 7.4, was heated at 65° C. for 5 minutes, then allowed to cool for an additional 5 minutes. MgCl₂ (10 mM) was then added for a total volume of 45 μL and the solution incubated for 10 minutes, followed by addition of 1.3 mM of the biotinylated small-molecule probe in DMSO. After incubation for the desired time, the RNA was precipitated by the addition of 5 μL of 3 M NaOAc and 180 μL of EtOH.

Dynabeads MyOne Streptavidin C1 coated beads (200 μg; Life Technologies) were prepared according to the instruction manual. The beads were resuspended in 20 μL of binding buffer (25 mM Na-HEPES, pH 7.4, 500 mM NaCl, 2.5 mM EDTA) and added to the RNA pellet. Following 25 minutes of room-temperature incubation on a rotation device, the beads were washed three times with 300 μL binding buffer, six times with 400 μL denaturing wash buffer (25 mM Na-HEPES, pH 7.4, and 5 mM EDTA in 8 M urea), three times with 300 μL H₂O, and then resuspended in 41.2 μL of H₂O.

To the RNA/bead solution was added 2 μM reverse transcription (RT) primer (GCCGCGAATTCACTAGTGATT). The solution was incubated at 65° C. for 5 minutes and room temperature for 3 minutes. 43.8 μL of RT solution (RT solution: 1.25 mM dNTPs (NEB), First Strand Buffer (Invitrogen), 230 mM DTT). Following removal of 19 μL of the mixture for a no-enzyme negative control, 4 μL of SuperScript III (Invitrogen) was added and the mixture incubated at 55° C. for 90 minutes.

The RT mixture was incubated with 35 μL of base-hydrolysis solution (80 mM Tris base, 15 mM EDTA, 1.25 M KOH) at 95° C. for 15 minutes and adjusted to pH 8.0 with 1 M HCl. 2 μL of this mixture was used in the subsequent PCR step using Taq DNA Polymerase (NEB).

Streptavidin Gel Mobility Shift Assays.

The pellet resulting from EtOH precipitation of a reaction of RNA (750 ng) with a small-molecule probe was resuspended in 21 μL H₂O. The RNA (7 μL) and streptavidin (NEB; 1 μg) were incubated at room temperature for 25 minutes and then combined with gel electrophoresis loading buffer. The sample was electrophoresed on a 10% PAGE denaturing gel (240 V, 35 minutes) and visualized following incubation with SYBR Green II (Life Technologies).

Kinetic Characterization of 42-nt A. Pernix Catalytic RNA.

At substrate concentrations greater than 8 mM, low levels of RNA modification were observed, potentially due to substrate aggregation. To calculate an upper limit for the Kₘ value, we fit a classic Michaelis-Menten equation to the experimentally derived data and additional values at very large substrate concentrations (more than 100-fold the calculated Kₘ) that would be expected to result in complete RNA modification.

Analysis of High-Throughput Sequencing.

To determine the fragment abundance in the round 5 cDNA library pool, the 80 bases after the constant primer sequence (TAGGCCGCGGGAATTCGATT) were identified. Sequences with Illumina Base quality scores of A through J at more than half of the positions across this 80-nt sequence were considered further. Sequences related by 12 or less mutations in this 80-nt sequence were binned and considered to have originated from the same fragment in the library. To determine the reselected variants from the partially randomized RNA pool derived from the minimized 42-nt A. pernix catalytic RNA, 42-nt sequences between the two constant primer sequences (TAGGCCGCGGGAATTCGATT and AATCACTAGTGAATTCGC) were identified. Only sequences with Illumina Base scores of B through J at more than half of the positions between the two primer sequences were considered further.

Determination of the Nucleotide Position of Epoxide-Catalytic RNA Modification.

The canonical 42-nt A. pernix catalytic RNA and a variant featuring a single base substitution (G¹⁰ to A¹⁰) (see below) was incubated with epoxide-alkyne 14. 5′-GGCAAAG⁷A⁸G⁹N¹⁰G¹¹CCCTGGGGTATGGAAGGGCTAGGCTCGTTGT-3′

Following EtOH precipitation, the RNA was resuspended in 50 mM NH₄OAc, pH 6 (340 μL) and incubated with 1,500 U RNase T1 (Roche) at 37° C. for 1 hour. Following lyophilization, the powder was resuspended in 45 μL H₂O and analyzed by LC/MS as described above.

RNase T1 cleaves the 3′-phosphodiester bond of unmodified guanosine nucleotides, but appears to have altered cleavage specificity for epoxide-modified G. Digestion of the canonical 42-nt A. pernix catalytic RNA (N¹⁰=G), following modification by epoxide 9, yielded m/z fragments corresponding to GG-epoxide ([M−H]⁻ m/z=961.3) and AGG-epoxide ([M−H]⁻ m/z=1290.4). These ions are consistent with epoxide reaction at either G⁹ or G¹⁰ to yield the 3-nt, epoxide-modified fragment below (AGG-epoxide), which undergoes further partial digestion between A⁸ and G⁹ to yield the GG-epoxide fragment:

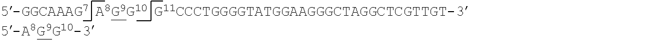

A similar analysis of the mutant in which N¹⁰=A established epoxide reaction at G⁹. RNase T1 digestion yields fragments corresponding to GA-epoxide ([M−H]⁻ m/z=945.3), AGA-epoxide ([M−H]⁻ m/z=1274.4), GAG-epoxide ([M−H]⁻ m/z=1290.4), and AGAG-epoxide ([M−H]⁻ m/z=1619.5), were observed. The change in observed products establishes that modification occurred at a nucleotide adjacent to N¹⁰ and is consistent with reaction at G⁹. The observed ions can be explained by canonical RNase T1 cleavage at the 3′-phosphodiester bond of unmodified guanosine nucleotides to yield the 4-nt fragment below (AGAG-epoxide), followed by cleavage between A⁸/G⁹ to yield GAG-epoxide, between A¹⁰/G¹¹ to yield AGA-epoxide, and at both A⁸/G⁹ and A¹⁰/G¹¹ to yield GA-epoxide.

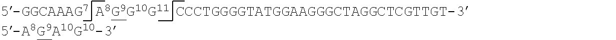

Synthesis and Characterization of the Authentic Chemical Standard.

The epoxide substrate 9 and guanosine 5′-monophosphate disodium salt hydrate (GMP; Sigma Aldrich) were combined in equimolar quantities (0.2 mmole) in glacial acetic acid (1.5 mL) and heated at 37° C. for 7 hours.⁴⁴ After allowing the mixture to cool, the acetic acid was removed at reduced pressure and the resulting residue was resuspended in H₂O and purified by reverse-phase HPLC (Agilent 1200) using a C18 stationary phase column (Eclipse-XDB C18, 5 μm, 9.4×200 mm) and acetonitrile/triethylammonium acetate (0.1 M) gradient.

To cleave the ribose from the modified guanine base, the epoxide-GMP product was heated in 1 M HCl (2 mL) at 95° C. for 7 hours, which also resulted in hydrolysis of the ester functionality. The mixture was neutralized with ammonium hydroxide and then lyophilized. The resulting residue was resuspended in H₂O and purified by HPLC.

Catalytic RNA Construct for RNA Labeling.

The catalytic RNA was cloned into the anticodon loop of a tRNA scaffold.⁴⁵ For the 5S rRNA, we employed a previously published construct containing a U5 transcription termination signal and the endogenous mammalian 5S promoter,⁴⁶ analogous to a recent report detailing 5S rRNA imaging using a fused aptamer.⁴⁷ The tRNA-optimized catalytic RNA sequence is as follows, with the optimized catalytic RNA underlined:

GCCCGGAUAGCUCAGUCGGUAGAGCAGCGGCCGCUCCAGAAGAGGGCCCC
UUCGGGGGCUAGGCUCGAUGUCGGCCGCGGGUCCAGGGUUCAAGUCCCUG
UUCGGGCGCCA

The construct containing three tandem copies of the catalytic RNA was as follows:

GCCCGGAUAGCUCAGUCGGUAGAGCAGCGGCCGAAUCUACUUAGAGGCCG
GAUUCUCCAGAAGAGGGCCCCUUCGGGGGCUAGGCUCGAUGUAAUCCGGC
CGCAGGUCGACUCUAGAAACGGAUACUUAGAGGCCGGAUUCUCCAGAAGA
GGGCCCCUUCGGGGGCUAGGCUCGAUGUAAUCCGGCCGCAGGUCGACUCU
AGAAAGUCUUACUUAGAGGCCGGAUUCUCCAGAAGAGGGCCCCUUCGGGG
GCUAGGCUCGAUGUAAUCCGGCCGCAGGUCGACUCUAGAAACUUACCGCG
GGUCCAGGGUUCAAGUCCCUGUUCGGGCGCCA

Transfection of Mammalian Cells and Enrichment of Total RNA.

Human embryonic kidney cells (HEK 293T) were obtained from ATCC and maintained in Dulbecco's modified Eagle medium (DMEM, Life Technologies) supplemented with 10% (vol/vol) fetal bovine serum (FBS, Life Technologies) and penicillin/streptomycin (1×, Amresco). Cells at ˜75% confluency were transfected 1 day after plating in 10 cm² plates (Greiner Bio-One) with 50 μL Lipofectamine 2000 (Life Technologies) and 15 μg of plasmid DNA. 2-3 days following transfection, total RNA was isolated by addition of TRIzol (Life Technologies) and subsequent use of RNeasy Mini spin columns (Qiagen).

Labeling and Enrichment of Catalytic RNA-Fusion Transcripts in Total RNA.

Total RNA from HEK 293T cells (10 μg) was incubated with the biotin-epoxide (1.3 mM) or the azide-epoxide (1.3 mM) as described above for 6 hours. Following EtOH precipitation, the azide-epoxide RNA was resuspended in 30 μL PBS and incubated for 1-3 hours with TAMRA-DBCO (15 μM; Click Chemistry Tools). The resulting solution was passed through two successive CENTRI•SEP Spin Columns (Princeton Separations) equilibrated with water and one RNEasy MinElute Column (Qiagen) to remove free TAMRA-DBCO. RNA concentration was quantified, normalized for all the samples, separated on a 5% PAGE-urea gel, and fluorescence was visualized using a Typhoon TRIO Variable Mode Imager (λₑₓ=532 nm, λₑₘ=580 nm).

For RT-qPCR quantification, the RNA pellet was resuspended in 50 μL H₂O. RNA was incubated with 200 μg Dynabeads MyOne Streptavidin C1 coated beads (Life Technologies) in either binding buffer (the pulldown material; 1 μg RNA) or H₂O (the no-pulldown material, 50 μg RNA). The samples were incubated at room temperature for 25 minutes, then washed three times with 300 μL binding buffer, six times with 500 μL denaturing wash buffer, and three times with 300 μL H₂O. The beads were then resuspended in 10 μL H₂O. On-bead reverse transcription was performed using 200 U Protoscript II (NEB) and 6 μM random primers (NEB) according to the manual. The RT mixture was incubated for 7 minutes at 25° C. and 75 minutes at 42° C. RNase H (5 U; NEB) was then added and the sample incubated at 37° C. for 20 minutes. qPCR was performed using 1 μL of cDNA template and 24 μL of iTaq Universal SYBR Green Supermix qPCR mix (Bio-Rad). Quantitative PCR was performed on a CFX-96 Real-Time System with a C100 Thermocycler (Bio-Rad).

As described in the main text, enrichment values were determined by comparing the ΔC_{T} of the catalytic RNA samples with the ΔC_{T} of the inactive mutant RNA, where the ΔC_{T} corresponds to the difference between the experimental sample and a control sample lacking streptavidin-linked bead capture. The ΔΔC_{T} values were normalized by the ΔΔC_{T} values of the housekeeping genes HPRT1 and tubulin. The average ΔC_{T} values for each of the cDNAs of interest are shown in Table 1.

TABLE 1
Quantification of RNA enrichment from total RNA.

| | 5S rRNA | HPRT1 | tubulin |
|---|---|---|---|
| inactive ribozyme | 7.49 | 5.38 | 5.00 |
| one copy of ribozyme | −0.06 | 4.66 | 4.56 |
| three copies of ribozyme | −1.81 | 5.23 | 4.71 |

ΔC_{T} values for RNA enrichment of the 5S rRNA, HPRT1 control, and tubulin control, as determined by RT-qPCR. The ΔC_{T} values represent the difference between the experimental sample and a control sample lacking streptavidin-linked bead capture.

Labeling and Enrichment of Catalytic RNA-Fusion Transcripts in Cell Lysate.

HEK 293T cells were cultured in 15 cm² plates as described above, washed once with 7.5 mL PBS, then removed from the plate in a total of 1 mL of PBS using a rubber1-headed plate scraper. The cells were then lysed by passing through a QiaShredder spin column (Qiagen), followed by addition of 80 U murine RNase inhibitor (NEB). Cell lysate (15 μL) was combined with 1×PBS (215 μL), 20 mM NaCl, 25 mM Na-HEPES, pH 7.4, and MgCl₂ (10 mM) and incubated at room temperature for 10 minutes, followed by addition of biotin-epoxide probe 1 (1.33 mM). The solution was incubated at room temperature for 6 hours. TRIzol LS (750 μL; Life Technologies) was added and RNA was isolated according to the TRIzol LS product instructions.

Pulldown of ASH1 mRNA-Binding Proteins.

Catalytic RNA-fused ASH1 mRNA was transcribed in vitro following the protocol provided in the T7 High Yield RNA Synthesis Kit (NEB). The RNA was reacted with biotin-epoxide for 24 hours and then precipitated with ethanol as described above. The biotinylated mRNA was immobilized on 400 μg Dynabeads MyOne Streptavidin C1 coated beads (Life Technologies) as follows: yeast tRNA (100 μg/mL; Life Technologies), 10 mM Tris-HCl, pH 7.5, 150 mM NaCl, and 5 μg of labeled RNA (total volume 49.5 μL) were incubated at 65° C. for 5 minutes and then cooled at room temperature for 5 minutes. MgCl₂ (10 mM) was then added and the solution was incubated at room temperature for 10 minutes to enable RNA folding. The streptavidin-coated magnetic beads were prepared according to the product manual, but washed twice with 10 mM Tris-HCl, pH 7.4. The supernatant was removed and the beads resuspended in the RNA solution. The RNA-bead mixture was rotated at room temperature for 1 hour.

TAP-tagged yeast strains from the yeast genome-wide TAP-tagged library⁴⁸ were cultured in YEPD medium penicillin/streptomycin (1×, Amresco), pelleted, and resuspended in 1.5 mL lysis buffer (10 mM Tris-HCl, pH 7.5, 150 mM NaCl, 2 mM MgCl₂, 0.5% v/v Triton-X 100, 1 mM DTT, and cOmplete, Mini, EDTA-free protease inhibitor (Roche)). The resuspended cells (900 μL) were combined with 0.5 mm silica-zirconia beads (Biospec Products) in a screw cap vial and bead beaten eight times at one-minute intervals on a Minibeadbeater (Biospec Products). After pelleting cellular debris, the supernatant (50 μL) was combined with the RNA-magnetic beads and the volume adjusted to 300 μL with lysis buffer. Solutions were incubated on a rotation device at 4° C. for 12 hours. The beads were then washed five times with 1 mL wash buffer (identical to lysis buffer but without protease inhibitor) and resuspended in 12 μL H₂O and 4 μL load dye. After heating at 95° C., the supernatant was loaded onto a 4-12% NuPAGE Bis-Tris Mini Gel (Life Technologies), which was run in MES buffer for 65 minutes at 150 V. Analysis was then performed by Western Blot.

### Results

Catalytic RNA-probes were used to identify reactive genome-encoded RNAs, resulting in the discovery of a 42-nt catalytic RNA from an archaebacterium that reacts with a 2,3-disubstituted epoxide at N7 of a specific guanosine. Detailed characterization of the catalytic RNA revealed the structural requirements for reactivity. We developed this catalytic RNA into a general tool to selectively conjugate a small molecule to an RNA of interest. This strategy enabled up to 500-fold enrichment of target RNA from total mammalian RNA or from cell lysate. We demonstrated the utility of this approach by selectively capturing proteins in yeast cell lysate that bind to the ASH1 mRNA.

We identified several electrophilic small molecules tuned to be unreactive to most RNA molecules, but to react with RNAs containing unusually nucleophilic functional groups. We used these probes to perform the in vitro selection of unusually reactive genome-encoded RNA fragments, resulting in the identification of a novel 42-nt catalytic RNA from the thermophilic archaea Aeropyrum pernix. This RNA catalyzes the nucleophilic attack of a guanosine onto a disubstituted epoxide that is unreactive to other RNAs, resulting in irreversible C—N bond formation. After establishing a minimal reactive motif and performing a bioinformatic search for examples in other organisms, we identified numerous examples of this unusually reactive RNA encoded in the mouse genome.

Recognizing the potential utility of a self-labeling RNA that reacts with a bioorthogonal small molecule, we developed this catalytic RNA into a tool for selective and irreversible RNA modification. The resulting RNA enables the covalent conjugation of diverse small-molecules to an RNA of interest and provides a robust handle for selective RNA capture from total RNA or even from cell lysate. We applied this tool to capture the ASH1 mRNA and enrich three known ASH1 mRNA-binding proteins from yeast cell lysate.

### Development of Chemical Probes for Unusually Reactive RNAs.

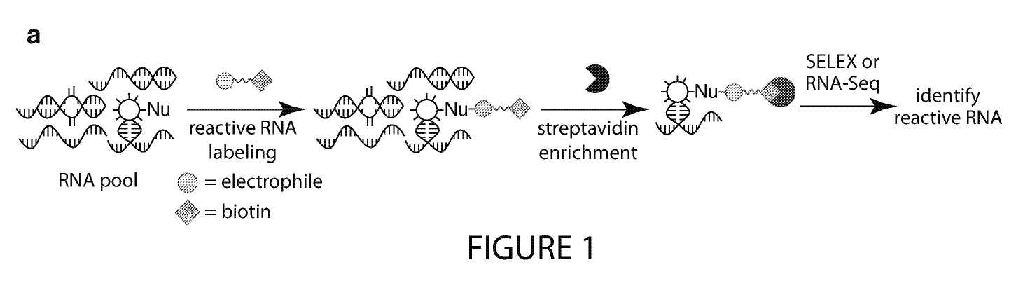

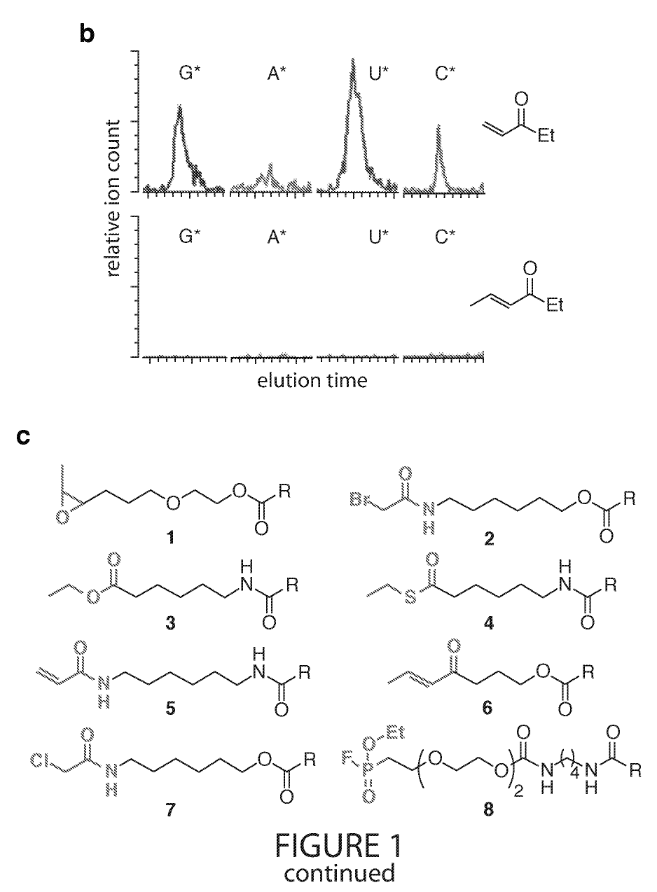

To develop activity-based chemical probes for RNA, we identified electrophilic small molecules capable of selectively and covalently labeling RNA functional groups with enhanced nucleophilicity, a feature shared among virtually all known RNA catalysts.^{4,15,21,22} We screened commercially available electrophilic small molecules for their inability to covalently modify an RNA pool of random sequence (N₈₀), representing typical, non-reactive RNA. This RNA pool was incubated with each electrophile, then digested to mononucleotides by nuclease P1 and characterized by liquid chromatography/mass spectrometry (LC/MS).²³ Electrophiles capable of efficiently modifying the random RNA pool (>10% of mononucleotides modified) were deemed too reactive (FIG. 1b). Instead, we focused on probe candidates that were insufficiently electrophilic to react with the random RNA pool to a detectable extent (<0.1%) (FIG. 1b).

Six electrophilic probes were identified from this screening approach: disubstituted epoxide 1, α-bromo acetamide 2,²⁴ ester 3, thioester 4, acrylamide 5, and disubstituted α,β-unsaturated ketone 6 (FIG. 1c). In addition, we identified two small molecules that have no detectable reactivity with the random RNA pool and that have been previously used for protein activity-based profiling: α-chloro acetamide 7,^{15,25,26} and fluorophosphonate 8.²⁷ Each of these eight probes was synthesized in a biotinylated form to enable streptavidin affinity capture of RNAs that form covalent bonds with the probes (FIG. 1c).

### Identification of Epoxide-Reactive Catalytic RNAs

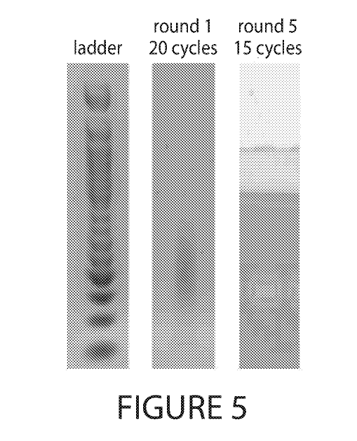

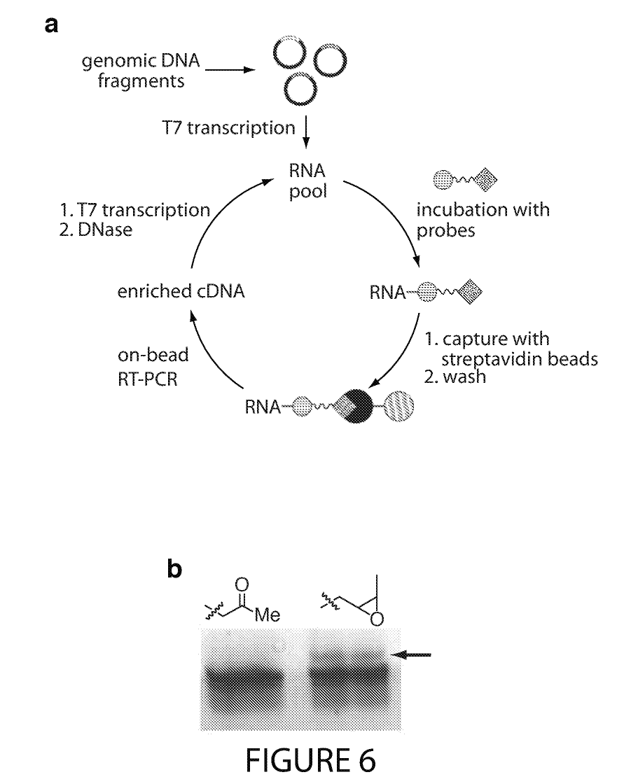

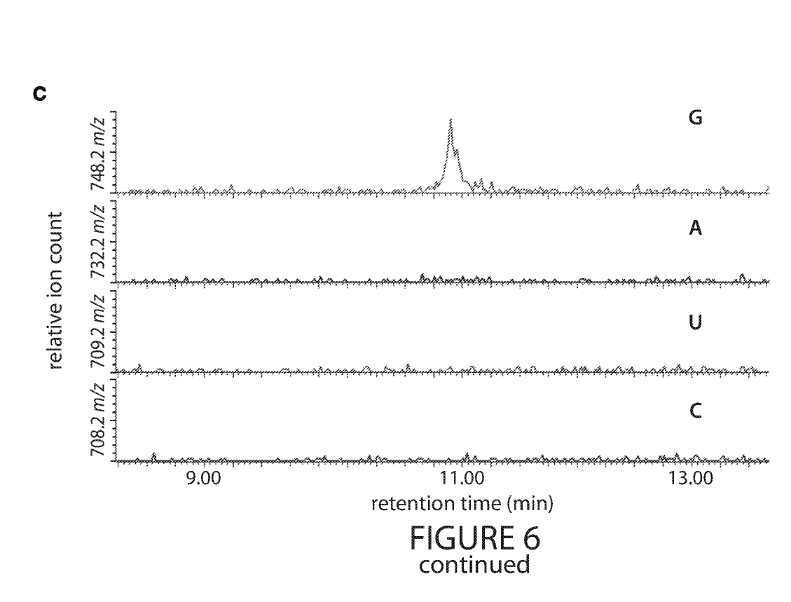

The eight biotinylated probes were combined into one cocktail and incubated with a pool of genome-derived RNA fragments from nine different organisms spanning all three kingdoms of life. Following five rounds of in vitro selection for binding to immobilized streptavidin, we observed enrichment of a substantial portion of the RNA pool and apparent convergence on discrete RNA species (FIGS. 5 and 6a). These results establish that unusually reactive RNA species exist within genome-encoded RNA pools and can be isolated using small-molecule probes of appropriate electrophilicity.

To identify the electrophile(s) that reacted with the enriched RNA, we performed one additional round of in vitro selection and then incubated the resulting post-round 6 RNA pool with each of the eight electrophilic probes. Streptavidin incubation and gel mobility shift assays revealed that one or more species of the post-round 6 RNA pool reacted with the disubstituted epoxide probe 1 (FIG. 6b). Nuclease P1 digestion and LC/MS analysis resulted in a mass spectrum consistent with reaction of the epoxide probe with a guanosine nucleobase to yield the corresponding epoxide ring-opened product (FIG. 6c).

To identify the reactive RNA, we subjected the round 6 DNA pool to high-throughput sequencing. We transcribed in vitro in the absence of flanking primer-binding sites the three most abundant round 6 library members and assayed their reactivity with epoxide probe 1 by gel mobility shift. The most abundant species (40% of reads) was a 125-nt fragment from two distinct regions of the B. fragilis genome, likely the result of ligation of two separate DNA fragments during construction of the genome-encoded RNA library. Although the full RNA reacted with epoxide probe 1, the individual genome-encoded fragments did not react with the probe. The second most abundant species (6.5% of reads) was a 52-nt fragment of the M. jannaschii genome. This RNA species showed only low levels of reactivity with the epoxide probe, suggesting that its reactivity with the probe is dependent on the specific sequence context of the selection. The third most abundant species (4.4% of reads) was a 63-nt fragment of the thermophilic archaea Aeropyrum pernix. This fragment catalyzed the epoxide ring-opening alkylation reaction in the presence or absence of the primer binding sites (FIG. 2a), indicating context independence.

Characterization of the A. pernix Catalytic RNA

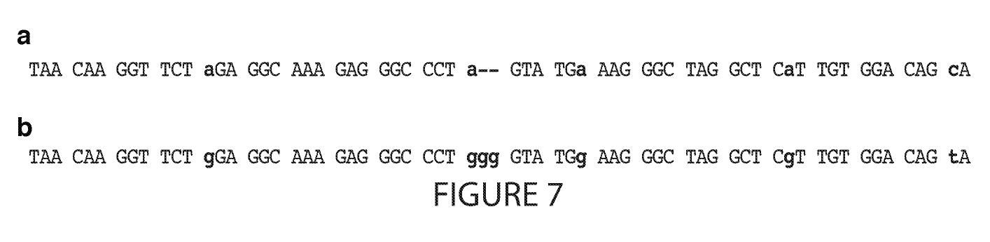

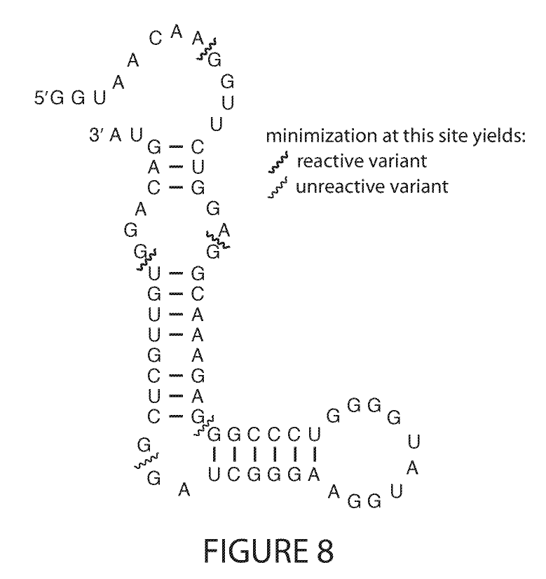

The A. pernix RNA sequence from the round 6 selection contained seven mutations or deletions compared with the A. pernix reference genome sequence (FIG. 7).²⁸ These mutations may have arisen from differences between the genomic DNA used to create the RNA pool and the reference genome, or from errors introduced during PCR amplification. To determine if these differences affect epoxide-opening activity, we generated an A. pernix fragment corresponding to the reference genome sequence, and observed no significant change in self-alkylation activity (FIG. 2a). Next we minimized the catalytic RNA by generating and assaying progressive 5′ and 3′ truncations (FIG. 8), resulting in a minimized 42-nt catalytic RNA with activity similar to that of the round 6 sequence.

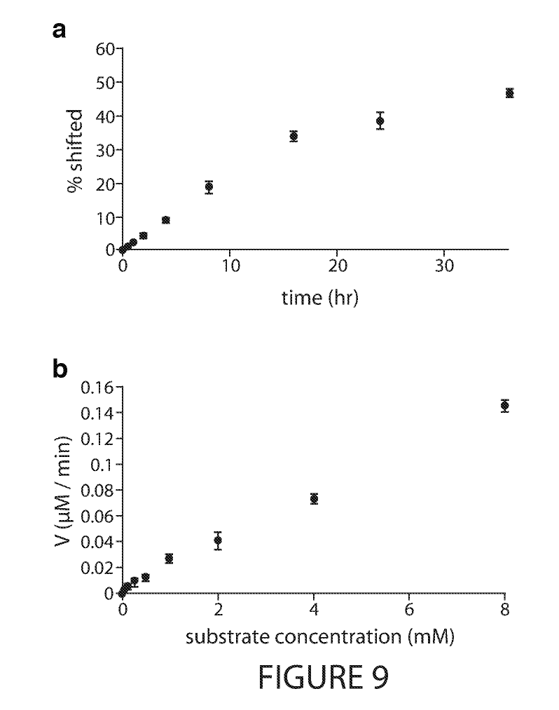

The minimized 42-nt A. pernix RNA exhibited a k_{cat} of 1.6×10⁻³ min⁻¹ and Kₘ for epoxide probe 1 of ≦0.012 M (k_{cat}/Kₘ≧0.13 min⁻¹ M⁻¹) (FIG. 9). The k_{cat}/Kₘ for the epoxide reaction of the minimized 42-nt RNA is at least 1,900-fold higher than that of a 42-nt pool of random-sequence RNA, which showed no detectable reaction even after incubation with 8 mM 1 for 60 hours. This catalytic efficiency is comparable to that of many known in vitro selected catalytic RNAs,²⁹⁻³² and represents the first reported example of a catalytic RNA capable of promoting epoxide ring opening.

LC/MS and NMR spectroscopic characterization of the product of the RNA-catalyzed epoxide ring opening revealed that N7 is the site of guanosine modification, similar to previous reports of DNA reacting with activated epoxides.^{33,34}

### Structural Optimization of the Epoxide Probe

To identify RNA-probe interactions that mediate probe reactivity, we investigated the structural requirements of the disubstituted epoxide substrate by synthesizing and assaying a series of epoxide analogs. While removal of the biotin group did not affect probe reactivity, shortening the alkyl chain reduced activity (FIG. 3). Replacing the ester group with an amide similarly decreased reaction efficiency (FIG. 3). These results suggest that the RNA might form direct contacts with the ester group in our substrates. We also studied the effect of epoxide stereochemistry, and found that the cis epoxide reacted 22-fold faster with the RNA than the trans epoxide (FIG. 3b), demonstrating that the catalytic RNA is highly stereospecific with respect to the epoxide functional group. Together, these results suggest that catalysis is dependent on multiple, specific RNA-substrate interactions.

### Sequence Requirements of the A. Pernix Catalytic RNA

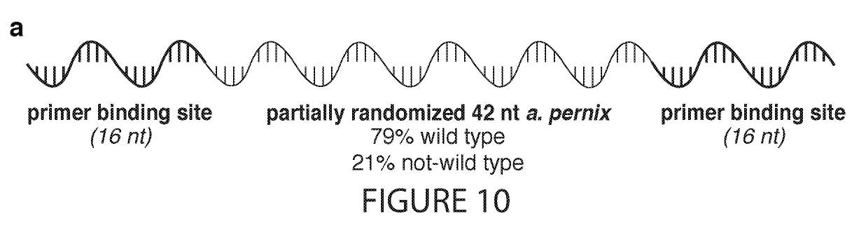

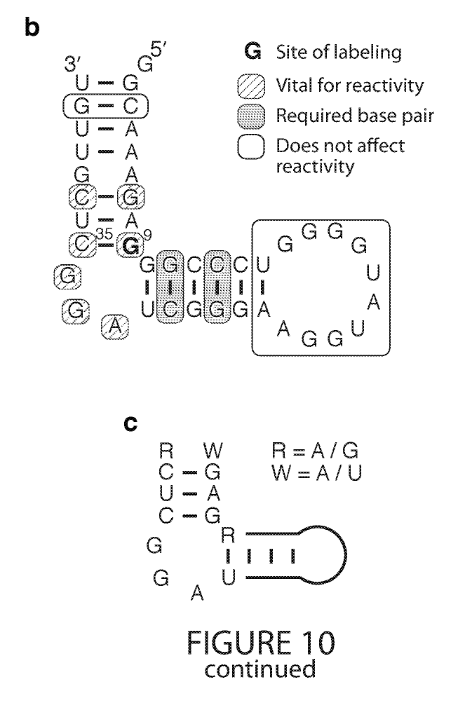

Computational secondary structure prediction³⁵ suggests that the A. pernix catalytic RNA folds into a stem-bulge-stem-loop structure (FIG. 2b). In order to probe this structural model and to identify the minimal sequence requirements for reactivity, we generated a partially randomized RNA pool derived from the minimized 42-nt A. pernix catalytic RNA and performed four rounds of reselection for epoxide reactivity (FIG. 10a). The enriched RNA pool was reverse transcribed and analyzed by high-throughput sequencing, resulting in the sequence logo shown in FIG. 2c.³⁶ The results are consistent with the predicted stem-bulge-stem model, reveal a highly conserved bulge region, and suggest that the 10-nt loop can be mutated without substantial loss of activity (FIG. 2b). This structural model was also supported by the results of site-directed mutagenesis experiments (Table 2).

TABLE 2
Activity of A. pernix site-directed mutant RNAs.

| Mutation | % Shifted |
|---|---|
| No changes | 35 |
| C³-G | 29 |
| G⁴²-C | 24 |
| C³-G⁴² | 27 |
| G⁷-C | 0 |
| C³⁸-G | 0 |
| G⁷-C³⁸ | 0 |
| G⁹-C | 0 |
| C³⁵-G | 0 |
| G⁹-C³⁵ | 0 |
| G¹⁰-A | 33 |
| G¹⁰-C | 0 |
| G¹⁰-U | 0 |
| U³¹-C | 29 |
| G¹¹-C | 0 |
| C³⁰-G | 0 |
| G¹¹-C³⁰ | 33 |
| C¹³-G | 0 |
| G²⁸-C | 0 |
| C¹³-G²⁸ | 38 |
| A³²-C | 0 |
| A³²-G | 0 |
| A³²-U | 0 |
| G³³-C | 0 |
| G³³-A | 0 |
| G³³-U | 0 |
| G³⁴-C | 13 |
| G³⁴-A | 14 |
| G³⁴-U | 15 |
| randomized | 34 |
| 10 bp loop | |

Percentage of shifted RNA was determined by streptavidin gel mobility shift assay with biotin-epoxide 1.

### Epoxide-Reactive Catalytic RNAs are Encoded by the Mouse Genome

To evaluate the potential existence of this catalytic RNA in other organisms, we developed a reactive minimal motif based on the sequence logo and mutational studies (FIG. 10c) and performed bioinformatics searches for examples of the motif in the genomes of Saccharomyces cerevisiae, Escherichia coli, and mouse. Although no examples of this motif were discovered in the smaller S. cerevisiae and E. coli genomes, 233 occurrences were found in the mouse genome.

To establish the ability of the minimal motif to predict catalytic RNA activity and to test if the examples encoded by the mouse genome were bona fide epoxide-reactive RNAs, we transcribed in vitro 44 of the 233 mouse genome-encoded candidate catalytic RNAs, chosen at random, and assayed their reactivity with disubstituted epoxide 1 by gel mobility-shift assays. Of the 44 genomic variants that were tested, 14 (32%) showed substantial levels of reactivity with the epoxide compared to that of random RNA. These observations demonstrate that the minimal catalytic RNA motif can be used to identify other candidate catalytic RNAs and also establishes that multiple examples of this unusually nucleophilic RNA are encoded in the mouse genome.

### Development of the Catalytic RNA for Selective RNA Modification

In contrast to the large number of available methods that effect the selective covalent modification of proteins,^{9,10} few techniques exist to selectively modify a genetically encoded RNA of interest.¹⁶ Such a method would provide a valuable tool for applications including live-cell RNA imaging,^{37,17,18} identification of RNA-binding proteins,^{19,20} and studies of RNA degradation.³⁸⁻⁴⁰ An ideal tool would enable efficient, selective, and bioorthogonal modification with a small-molecule probe capable of carrying diverse chemical functionality including affinity handles, azide- or alkyne-containing chemical handles, and fluorophores. We speculated that the self-labeling A. pernix RNA might serve as such a tool for selective RNA modification in complex biological samples. In order to improve the rate of the epoxide-RNA reaction to facilitate efficient covalent RNA modification, we performed seven rounds of high-stringency reselections with decreasing epoxide incubation times on the partially randomized 42-nt A. pernix RNA library, resulting in an optimized catalytic RNA exhibiting a 5-fold faster rate of reaction (FIGS. 2d, 9a, 10, and 11b).

### Generality of the Epoxide Probe

The ability of the epoxide probe to support catalytic RNA-mediated RNA modification with diverse chemical groups was evaluated. Probes in which the biotin group was replaced with an azide, alkyne, carboxamide, alkyl chain, or tetramethylrhodamine (TAMRA) fluorophore were synthesized and tested for their ability to react with the optimized catalytic RNA (FIGS. 3c and d). All of the probes assayed remained efficient RNA substrates, suggesting that this system can mediate a diverse range of RNA functionalization.

### Catalytic RNA-Mediated RNA Enrichment

We integrated the above findings to test the ability of the optimized catalytic RNA when transcriptionally fused to an RNA of interest to enable enrichment of that RNA from a complex biological mixture. We cloned the optimized catalytic RNA at the 3′-end of the human 5S rRNA and generated the resulting RNA using T7 RNA polymerase. Following 8-hour incubation with epoxide probe 1, we evaluated modification of the RNA by gel mobility shift, revealing reaction efficiency (35%) similar to that of the unfused catalytic RNA (40%). This observation demonstrates that the optimized catalytic RNA can retain its activity when appended to an unrelated RNA of interest.

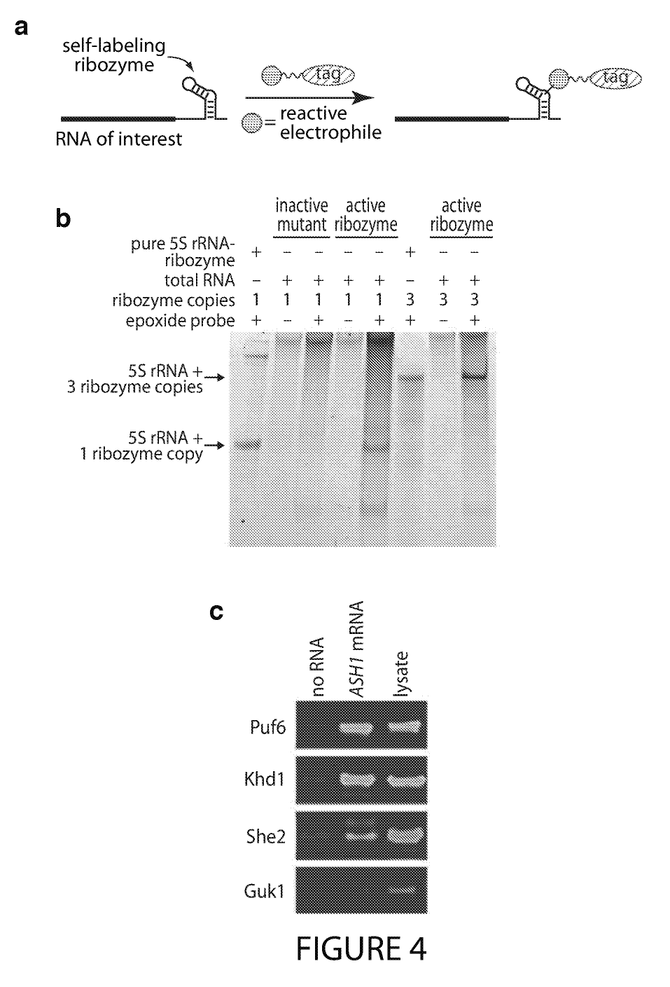

To establish the ability of the catalytic RNA to enrich an RNA of interest from total cellular RNA, we transfected the 5S rRNA-catalytic RNA construct into HEK 293T cells and isolated total RNA. As a control we used a construct in which the reactive G⁹ and base C³⁵, predicted to pair with G⁹, were swapped to yield an inactive isomeric mutant catalytic RNA predicted to adopt the same secondary structure. Total cellular RNA was incubated for 5 hours with azide epoxide 14, followed by copper-free click chemistry using dibenzocyclooctyne-TAMRA (DBCO-TAMRA) to install a TAMRA fluorophore and PAGE analysis (FIG. 4b). While no TAMRA-bound 5S rRNA product was generated from the mutant catalytic RNA, we observed a strong fluorescent band of the expected size for the sample containing the active catalytic RNA (FIG. 4b). Repeating this experiment with a construct containing three consecutive copies of the catalytic RNA (5S rRNA-catalytic RNA₃) resulted in 3.2-fold higher fluorescence signal (FIG. 4b).

To quantify the ability of the catalytic RNA to enrich an RNA of interest from total cellular RNA, we captured biotinylated RNA using immobilized streptavidin and quantified the amount of enriched catalytic RNA-fused 5S rRNA by reverse transcription-quantitative PCR (RT-qPCR). We isolated total RNA from HEK 293T cells transfected with a vector expressing the human 5S rRNA transcriptionally fused to either one or three copies of the catalytic RNA, or fused to the inactive mutant catalytic RNA. Total cellular RNA from each sample was incubated with epoxide probe 1 and captured with streptavidin-linked magnetic beads. Following on-bead reverse transcription, we performed qPCR to quantitate the extent to which the catalytic RNA-fused transcript was enriched over an inactive transcript and observed 125- and 541-fold enrichment for transcripts fused to one and three copies of the catalytic RNA, respectively (Table 1).

We attempted to selectively modify and isolate a catalytic RNA-fused transcript from total human cell lysate. HEK 293T cells expressing 5S rRNA-catalytic RNA transcripts were lysed and incubated with epoxide probe 1. Total RNA was isolated and the efficiency of reaction with the epoxide probe was quantified by RT-qPCR (following enrichment with immobilized streptavidin). The 5S rRNA transcript fused to one or three copies of the catalytic RNA was enriched 57-fold and 398-fold, respectively, over the inactive variant. These findings establish that catalytic RNA-fused transcripts are selectively modified even in mammalian cell lysate, enabling their facile isolation.

### Unbiased Enrichment of RNA-Binding Proteins

The ability to selectively and covalently modify and immobilize an RNA of interest in principle enables the rapid isolation of RNA-binding proteins.^{19,20} To demonstrate this capability, we transcribed in vitro the well-characterized yeast mRNA ASH1⁴¹ with the optimized catalytic RNA inserted immediately upstream of the 3′-UTR. We immobilized the transcript by incubation with epoxide biotin probe 1 and capture with streptavidin-coated beads. We treated the immobilized ASH1 mRNA with yeast lysate from three strains individually expressing TAP-tagged proteins known to bind the ASH1 mRNA (Puf6, She2, and Khd1) and lysate from a fourth strain expressing TAP-tagged Guk1, which is not known to bind the ASH1 mRNA. Following extensive washing, binding proteins were eluted from the beads. Western blot revealed 29-fold average enrichment of the three known binding proteins relative to samples containing no RNA and no substantial enrichment of the non-binding protein Guk1 (FIG. 4c). These results demonstrate that the catalytic RNA-epoxide reaction enables site-specific biotinylation and immobilization of an mRNA of interest, followed by efficient capture of multiple proteins that bind the mRNA from cell lysate.

### DISCUSSION

The use of electrophilic chemical probes to discover unusually reactive RNAs provides an unbiased alternative to methods that rely on homology to known catalytic RNAs or on secondary structure prediction. To realize this potential, we identified electrophilic chemical groups with reactivity profiles biased towards reactive RNA modification, but insufficiently reactive to modify random RNA molecules. Although the reactivity of activated allylic or benzylic epoxides towards nucleic acids is well documented,^{33,34} to our knowledge the A. pernix catalytic RNA described in this work represents the first example of an RNA that catalyzes nucleophilic attack of an unactivated epoxide. The reaction occurs at a single guanosine base, resulting in C—N bond formation at N7.

Additional studies revealed that the A. pernix catalytic RNA requires only 42 nt for activity and likely adopts a stem-bulge-stem architecture with a stereospecific active site. Reselection using a partially randomized RNA pool established a sequence logo that was used to identify a minimal reactive motif and many naturally occurring transcripts capable of reacting with the epoxide. While many tested naturally occurring genome-encoded variants of the catalytic RNA proved to be active, it is possible that their activity is unrelated to any biological role. Elucidating their potential biological relevance, if any, will require future investigation.

The use of the transcriptionally fused catalytic RNA to modify an mRNA at a single position provides a unique tool for RNA labeling and the isolation of RNA-binding proteins. Standard approaches to isolating RNA-binding proteins involve in vitro transcription using biotinylated uracil,⁴² resulting in a heterogeneous mixture of biotinylated RNAs, or the use of a fused aptamer,¹⁹ which requires careful development of washing conditions to ensure maintenance of aptamer binding. Because the method described here establishes a robust covalent bond between the probe and the RNA, washing conditions can be vigorous; for example, RNA captured in this study survived successive washes with 8 M urea. The chemical orthogonality of the catalytic RNA-epoxide reaction is sufficient to enable selective covalent RNA modification in cell lysate, suggesting its value to studying RNAs of interest in native biological contexts. Moreover, the modularity of the epoxide probe suggests that cell-permeable analogues based on these developments may enable live-cell RNA imaging or RNA-protein crosslinking applications.

### In Vitro Selection of Nucleophilic, Genome-Encoded RNAs

The eight biotinylated probes were combined into one cocktail and incubated with a pool of genome-derived RNA fragments from nine organisms spanning all three kingdoms of life (Arabidopsis thaliana, Aeropryum pernix, Haloarcula marismortui, Methanococcus jannaschii, Bacillus subtilis, Bacteroides fragilis, Escherichia coli, Gallus gallus domesticus, and Homo sapiens). We recently discovered two families of naturally occurring GTP-binding RNAs by performing a selection on these RNA pools.^{49,50} The libraries were constructed by random DNase I-catalyzed fragmentation of genomic DNA and isolation of fragments between 100 and 600 bp by gel electrophoresis. These DNA fragments were transcribed by T7 RNA polymerase to yield genome-derived RNA fragments flanked by primer binding sites suitable for in vitro selection. Genome-encoded RNA pools constructed in this manner, as opposed to RNA isolated from cells, avoid the underrepresentation of many RNA species due to large expression-level differences and, in the case of RNA from multicellular organisms, due to tissue-specific transcription.

The mixture of in vitro-transcribed genome-encoded RNA fragments from all nine organisms together with the eight biotinylated probes was incubated in buffer chosen to approximate physiological conditions (25 mM Na-HEPES, pH 7.4, 150 mM NaCl, 10 mM MgCl₂). Biotinylated RNA fragments were isolated using streptavidin-coated magnetic beads, then subjected to on-bead reverse transcription followed by PCR amplification (FIG. 6a). Following five rounds of selection, amplification, and transcription, we observed enrichment of a substantial portion of the RNA pool. Agarose gel electrophoresis of the corresponding cDNA revealed discrete bands consistent with convergence to discrete RNA species (FIG. 5). Collectively, these results suggest that unusually reactive RNA species exist within diverse genome-encoded RNA pools and can be isolated using small-molecule probes of tuned electrophilicity.

### Sequence Requirements of the A. Pernix Catalytic RNA

Computational secondary structure prediction⁵¹ suggests that the A. pernix catalytic RNA folds into a stem-bulge-stem-loop structure (FIG. 2b). In order to probe this structural model and to identify the minimal sequence requirements for reactivity, we generated a partially randomized RNA pool derived from the minimized 42-nt A. pernix RNA and performed a reselection for epoxide reactivity (FIG. 10a). The RNA was incubated with the epoxide probe for 4 h each round and, following four rounds of selection, converged on a subset of reactive constructs (see Methods). The enriched RNA pool was reverse transcribed and analyzed by high-throughput sequencing, resulting in the sequence logo shown in FIG. 2c.⁵² This analysis supported the predicted stem-bulge-stem model, revealed a highly conserved bulge region, and suggested that the 10-nt loop could be mutated without substantial loss of activity.

The resulting structural model was further probed by site-directed mutagenesis. Nucleotides predicted to form a base pair were mutated to determine whether catalytic RNA activity tolerated swapping of the presumed base-pair nucleotides. Constructs featuring compensatory mutations at G¹¹-C³⁰ and C¹³-G²⁸ retained activity, whereas any mutation at G⁷-C³⁷ or G⁹-C³⁵ resulted in the complete loss of activity (FIG. 2b). The requirement for the presence of the predicted 10-nt loop was investigated by testing the reactivity of an RNA pool featuring a randomized loop, which exhibited reactivity with the epoxide comparable to that of the canonical catalytic RNA. We also probed the predicted bulge region that was highly conserved during reselection by testing the reactivity of all four possible nucleotides at these positions. Mutation of G¹⁰ to A did not significantly affect RNA reactivity, while C and U were not tolerated. All mutations at A³² and G³³ resulted in loss of RNA reactivity, while mutations at G³⁴ significantly decreased activity. Collectively, the activities of these mutant RNAs are consistent with the reselection results and support the structural model in FIG. 2b.

Metal Ion Requirements of the A. pernix Catalytic RNA

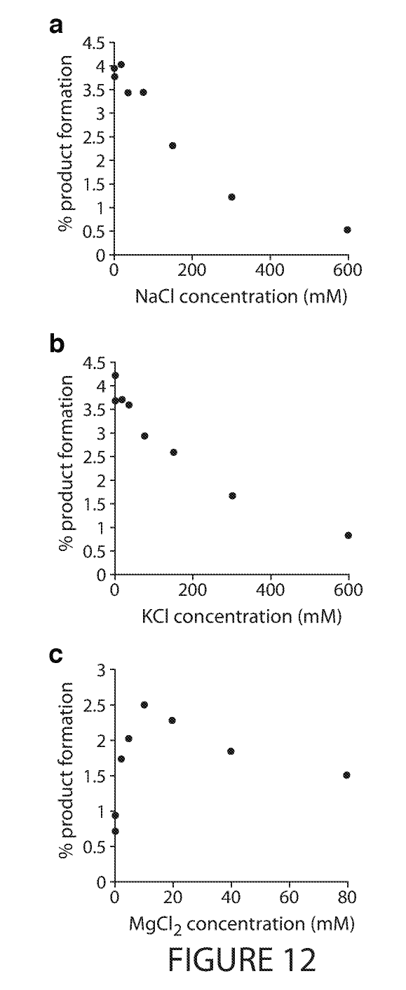

A common characteristic of functional RNAs is a dependence on metal cations.⁵³ The current selection was performed in the presence of Mg²⁺ and Na⁺. RNA activity was not substantially affected by concentrations of Na⁺ or K⁺ up to 75 mM (FIG. 12). In contrast, RNA activity was dependent on Mg²⁺, with optimal activity at 10 mM Mg²⁺ (FIG. 12). Testing other divalent cations instead of Mg²⁺ revealed that the RNA retains activity in the presence of Ca²⁺, Mn²⁺, or Co²⁺, albeit at slightly reduced rates, but not in the presence of Ni²⁺ or Zn²⁺.

### Characterization of Alkylation Regiochemistry

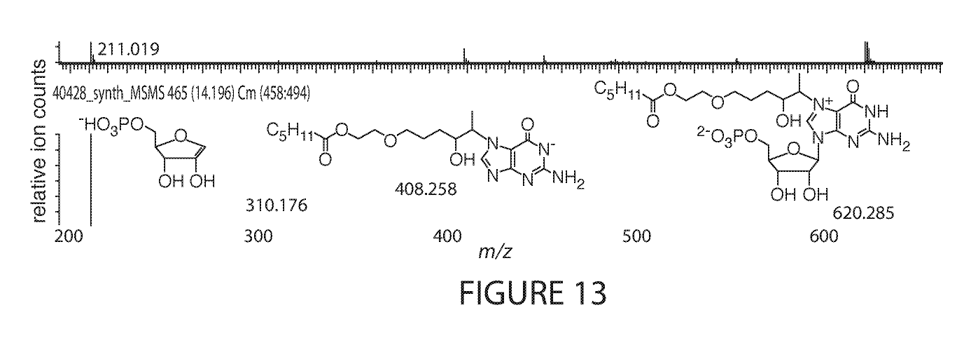

LC/MS analysis revealed that RNA modification by 1 occurs selectively on guanosine (FIG. 6c). To identify which guanosine was modified, the wild-type catalytic RNA and a panel of single-base mutants were incubated with the epoxide probe and then partially digested with RNase T1, an endonuclease that cleaves 3′ of guanosine residues. The resulting nucleotide fragments were characterized by LC/MS. This approach revealed G⁹ as the site of alkylation by 1 (see Methods). LC/MS/MS fragmentation of the ion corresponding to the epoxide modified guanosine mononucleotide resulted in fragments consistent with glycosidic bond fragmentation to yield ribose ([M−H]⁻ m/z=211.019) and a species corresponding to the product of a reaction between the epoxide and a guanine base ([M−H]⁻ m/z=408.258) (FIG. 13).

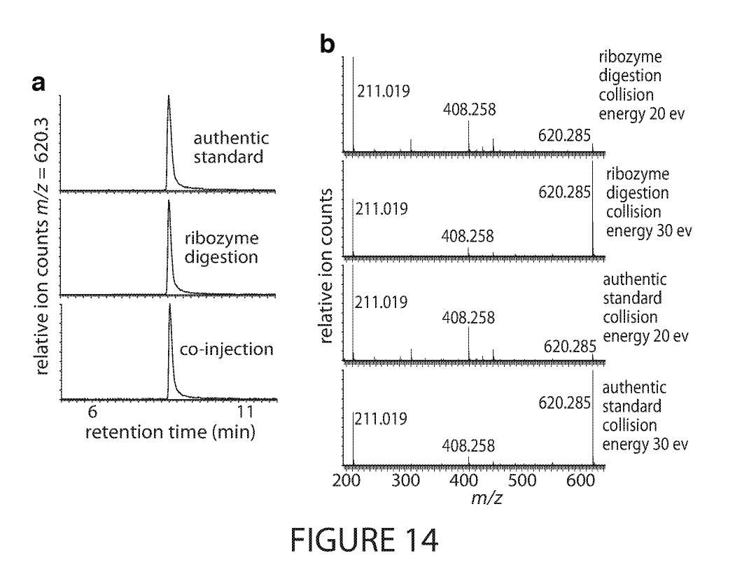

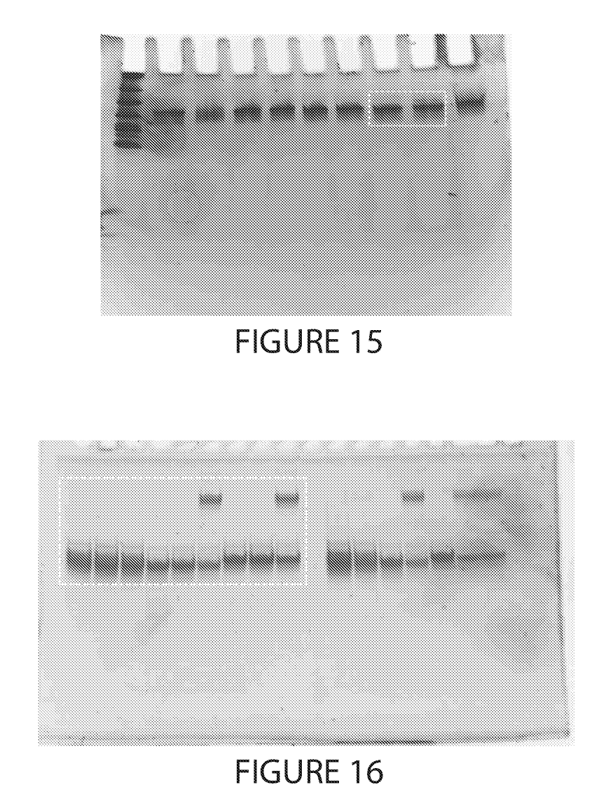

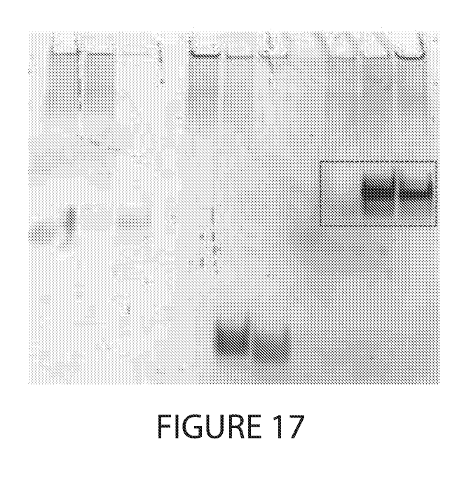

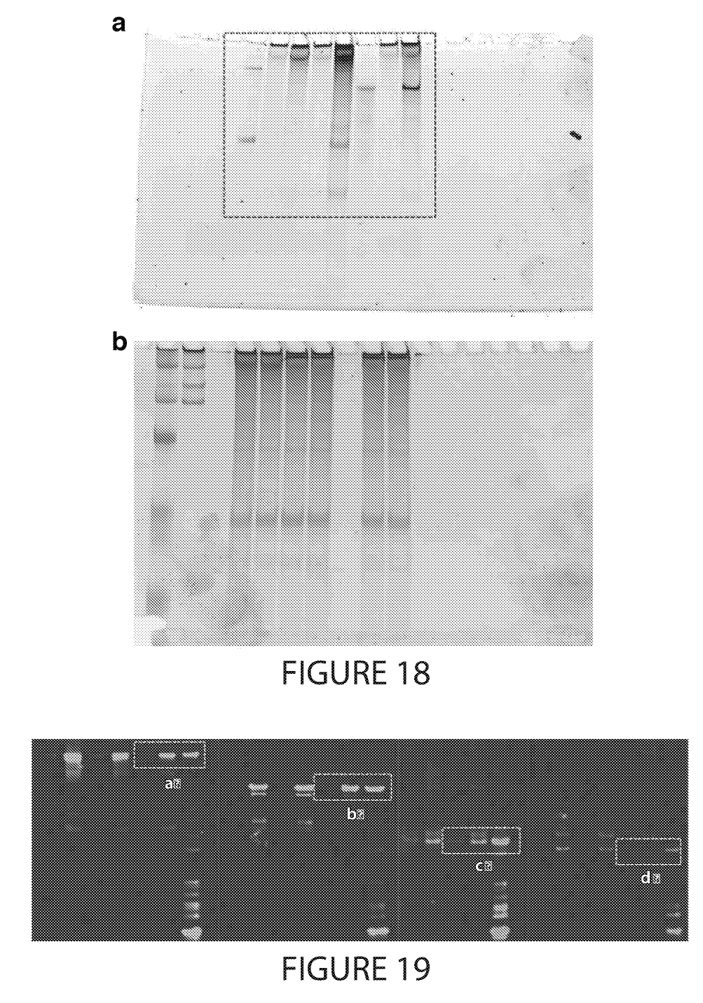

To elucidate the structure of the guanine epoxide adduct, we synthesized an authentic chemical standard by heating the epoxide probe and 5′-monophosphate guanosine in acetic acid and determined the structure of the major reaction product by NMR spectroscopy to be the adduct arising from bond formation between N7 of guanosine and the epoxide probe (FIG. 13). Comparison of this authentic synthetic product with the alkylated guanosine generated by P1 nuclease digestion of the catalytic RNA after incubation with the epoxide probe revealed identical LC retention times and MS/MS fragmentation patterns at varying collision energies (FIG. 14). These results indicate that N7 is the site of guanosine modification, similar to previous reports of DNA reacting with activated epoxides⁵⁴⁻⁵⁶ (see Methods for details).

### Optimization of the Catalytic RNA

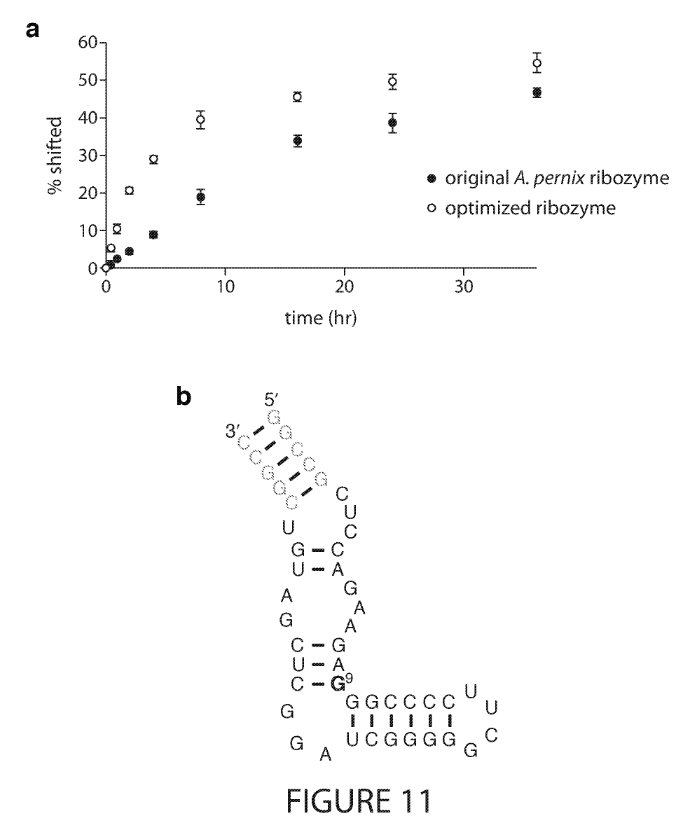

In order to improve the rate of the epoxide-RNA reaction to facilitate efficient covalent RNA modification, we used the partially randomized 42-nt A. pernix RNA library to perform high-stringency reselections with decreasing epoxide incubation times. After seven rounds of reselection, we characterized the resulting RNA pool by high-throughput sequencing. The ten most abundant library members were transcribed in vitro and their rates of epoxide self-labeling were assayed by gel mobility shift. Following selection of the most reactive candidates and additional minimization and engineering, we isolated a 41-nt variant containing four core mutations relative to the starting A. pernix catalytic RNA, a minimized 4-nt loop, and changes to the 5′- and 3′-ends (FIG. 11b). This optimized catalytic RNA exhibited a 5-fold faster rate of reaction with epoxide probe 1 (FIG. 11).

Quantification of RNA Enrichment from Total Cellular RNA

Following on-bead reverse transcription, we performed qPCR of the 5S rRNA and determined the difference in qPCR cycle threshold (ΔC_{T}) between the experimental sample and a control sample lacking streptavidin-linked bead capture for each of the three catalytic RNA fusion variants. To compare streptavidin capture of the 5S rRNA across the three samples, we calculated ΔΔC_{T} for each catalytic RNA sample compared to the inactive control, resulting in an average ΔΔC_{T} of 6.96 for the 5S rRNA containing one copy of the catalytic RNA and an average ΔΔC_{T} of 9.08 for three copies of the catalytic RNA. These values correspond to a 125- and 541-fold enrichment, respectively, of the catalytic RNA-fused transcript over the inactive transcript.

### Synthesis of Epoxide Probes

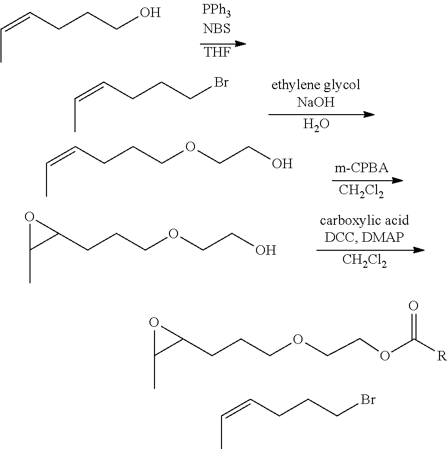

Triphenylphosphine (64 mmol; 1 equiv) was dissolved in dry THF (100 mL), followed by addition of cis-4-hexen-1-ol (64 mmol; 1 equiv). The solution was cooled in an ice bath, and then N-bromosuccinimide (67 mmol; 1.05 equiv) was added in portions. The solution was allowed to warm to room temperature and stirred overnight. After removing solvent at reduced pressure, the mixture was passed through a silica gel plug and flushed with hexanes (˜500 mL). Hexanes were removed at reduced pressure to yield a colorless oil (7.72 g; 74% yield). Characterization data matched literature data.⁵⁷

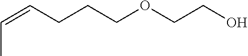

The alkyl bromide (28 mmol, 1 equiv) and ethylene glycol (224 mmol, 8 equiv) were combined in a round bottom flask. A solution of aqueous NaOH (112 mmol, 4 equiv in 6 mL H₂O) was then added slowly dropwise with vigorous stirring. The solution was submerged into a pre-heated 80° C. oil bath and stirred for 12 hours. After cooling to room temperature, the solution was diluted with H₂O and extracted three times with Et₂O. The combined organic layers were dried with MgSO₄ and the solvents removed at reduced pressure. The resulting oil was purified by silica gel chromatography (3:2 hexanes:EtOAc) to yield 2.18 g (54% yield) of a light-yellow oil. ¹H NMR (400 MHz, CDCl₃) δ 1.59 (d, J=6.7 Hz, 3H), δ 1.64 (p, J=6.7 Hz, 2H), δ 2.10 (q, J=6.7 Hz, 2H), δ 2.24 (bs), δ 3.47 (t, J=6.7 Hz, 2H), δ 3.52 (t, J=4.7 Hz, 2H), δ 3.71 (t, J=4.7 Hz, 2H), δ 5.32-5.40 (m), δ 5.41-5.51 (m). ¹³C NMR (100 MHz, CDCl₃) δ 129.75, δ 124.45, δ 71.78, δ 70.67, δ 61.81, δ 29.35, δ 23.32, δ 12.70. HRMS: m/z (ESI) calculated [M+H]⁻=143.1072, measured 143.1062 (Δ=7.0 ppm)

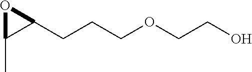

m-CPBA (4.6 mmol; 1.2 equiv) was dissolved in dry CH₂Cl₂ (8.5 mL) and cooled to 0° C. A solution of the alkene (3.8 mmol; 1 equiv) in CH₂Cl₂ (1.5 mL) was added dropwise via syringe. The solution was removed from the ice bath and stirred at room temperature. Following disappearance of the starting material, the solution was washed 2 times with aq. 10% Na₂CO₃ and once with brine. The organic layer was then dried with MgSO₄ and the solvent removed at reduced pressure. Silica gel column chromatography (3:2 EtOAc:Hexanes) yielded the desired product as a colorless oil (206 mg, 34% yield). ¹H NMR (400 MHz, CDCl₃) δ 1.26 (d, J=5.5 Hz, 3H), δ 1.50-1.70 (m, 2H), δ 1.72-1.83 (2H), δ 2.35 (bs, 1H), δ 2.90-2.94 (m, 1H), δ 3.02-3.08 (m, 1H), δ 3.53-3.56 (m, 4H), δ 3.69-3.74 (m, 2H). ¹³C NMR (100 MHz, CDCl₃) δ 71.84, δ 70.53, δ 61.74, δ 56.95, δ 52.69, δ 26.74, δ 24.45, δ 13.15. HRMS: m/z (ESI) calculated [M+H]⁻=159.1021, measured 159.1025 (Δ=2.5 ppm)

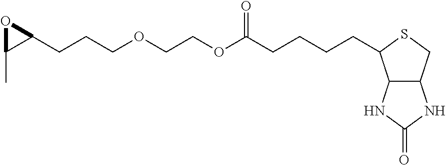

The epoxide (0.47 mmol; 1 equiv) was dissolved in CH₂Cl₂ (5 mL), followed by addition of D-biotin (0.52 mmol; 1.1 equiv). The solution was cooled to 0° C., followed by addition of N,N′-dicyclohexylcarbodiimide (DCC; 0.52 mmol, 1.1 equiv) and 4-dimethylaminopyridine (DMAP; 0.02 mmol; 0.05 equiv). The solution was removed from the ice bath and stirred overnight at room temperature. Following removal of the solvent at reduced pressure, the reaction mixture was purified by silica gel column chromatography (7.5% MeOH in CH₂Cl₂) to yield epoxide-biotin 1 as a colorless solid (66 mg; 55% yield). ¹H NMR (400 MHz, CDCl₃) δ 1.25 (d, J=5.6 Hz, 3H), δ 1.41-1.49 (m, 2H), δ 1.54-1.78 (m, 8H), δ 2.36 (t, J=7.4 Hz, 2H), δ 2.70 (d, J=12.8 Hz, 1H), δ 2.90-2.97 (m, 2H), δ 3.04-3.09 (m, 1H), δ 3.18-3.22 (m, 1H), δ 3.29-3.31 (m, 2H), δ 3.51-3.56 (m, 2H), δ 3.62-3.66 (m, 2H), δ 4.19-4.22 (m, 2H), δ 4.30 (dd, J=7.9, 4.6 Hz, 1H), δ 4.48 (dd, J=7.7, 4.6 Hz, 1H). ¹³C NMR (100 MHz, CDCl₃) δ 201.0, δ 173.8, δ 70.2, δ 68.3, δ 63.1, δ 61.9, δ 60.2, δ 56.8, δ 55.5, δ 52.6, δ 39.6, δ 33.3, δ 28.2, δ 26.1, δ 24.5, δ 23.9, δ 11.9. HRMS: m/z (ESI) calculated [M+H]⁺=387.1979, measured 387.1961 (Δ=4.6 ppm)

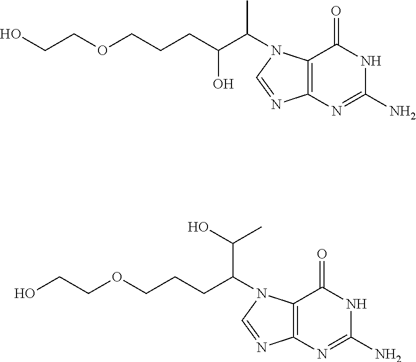 [SMILES: CC(C(O)CCCOCCO)n1cnc2nc(N)[nH]c(=O)c21 ; CC(O)C(CCCOCCO)n1cnc2nc(N)[nH]c(=O)c21]

¹H NMR (600 MHz, D₂O) δ 0.92 (d, J=6.45 Hz, 3H), δ 1.11 (t, J=7.4 Hz, 3H), δ 1.19-1.27 (m, 1H), δ 1.33-1.41 (m, 1H), δ 1.75-1.84 (m, 2H), δ 3.01-3.07 (m, 3H), δ 3.44 (t, J=4.5 Hz, 2H), δ 3.48-3.53 (m, 2H), δ 3.62 (p, J=6.3 Hz, 1H), δ 4.14 (t, J=6.8 Hz, 2H), δ 7.79 (s, 1H). ¹³C NMR (150 MHz, CD₃OD) δ 156.7, δ 155.6, δ 154.8, δ 142.5, δ 110.3, δ 86.2, δ 74.6, δ 70.9, δ 63.9, δ 63.5 δ 29.0, δ 28.8, δ 19.9.

¹H, ¹³C, and 1D NOESY NMR spectroscopy were used to establish that the reaction occurred at N7 of guanine. Through space coupling with the hydrogen at C8 of guanine was observed with epoxide-substrate hydrogens are indicative of N7 labeling. In addition to the spectroscopic evidence, reaction at positions other than N7 would lead to modification on the Watson-Crick face and would inhibit reverse transcription. This would prohibit propagation of the RNA through the in vitro selection.

### REFERENCES

1 Djebali, S. et al. Landscape of Transcription in Human Cells. Nature 489, 101-108 (2012).
2 Kowtoniuk, W. E. et al. A Chemical Screen for Biological Small Molecule-RNA Conjugates Reveals CoA-Linked RNA. Proc. Natl. Acad. Sci. USA 106, 7768-7773 (2009).
3 Dumelin, C. E.; Chen, Y.; Leconte, A. M.; Chen, Y. G.; Liu, D. R. Nat. Chem. Biol. 8, 913-919 (2012).
4 Doudna, J. A. & Cech, T. R. The Chemical Repertoire of Natural Ribozymes. Nature 418, 222-228 (2002).
5 Fedor, M. J. & Williamson, J. R. The Catalytic Diversity of RNAs. Nat. Rev. Mol. Cell Bio. 6, 399-412 (2005).
6 Joyce, G. F. Forty Years of In Vitro Evolution. Angew. Chem. Int. Ed. 46, 6420-6436 (2007).
7 Cravatt, B. F., Wright, A. T. & Kozarich, J. W. Activity-Based Protein Profiling: From Enzyme Chemistry to Proteomic Chemistry. Annu. Rev. Biochem. 77, 383-414 (2008).
8 Sadaghiani, A. M., Verhelst, S. H., & Bogyo, M. Tagging and Detection Strategies for Activity-Based Proteomics. Curr. Opin. Chem Biol. 11, 20-28 (2007).
9 Hinner, M. J. & Johnsson, K. How to Obtain Labeled Proteins and What to Do With Them. Curr. Opin. Biotechnol. 21, 766-776 (2010).
10 Jing, C. & Cornish, V. W. Chemical Tags for Labeling Proteins Inside Living Cells. Acc. Chem. Res. 44, 784-792 (2011).
11 The electrophile N-methylisotoic anhydride has found widespread utility for RNA structure determination: Low, J. T. & Weeks, K. M. Shape-Directed RNA Secondary Structure Predicition. Methods 52, 150-158 (2010)
12 Baruah, H., Puthenveetil, S., Choi, Y. A., Shah, S. & Ting, A. Y. An Engineered Aryl Azide Ligase for Site-Specific Mapping of Protein-Protein Interactions through Photo-Cross-Linking. Angew. Chem. Int. Ed. 47, 701-7021 (2008).
13 Rutkowska, A., Haering, C. H. & Schultz, C. A FlAsH-Based Cross-Linker to Study Protein Interactions in Living Cells. Angew. Chem. Int. Ed. 50, 12655-12658 (2011).
14 Chidley, C., Haruki, H., Pedersen, M. G., Muller, E. & Johnsson, K. A Yeast-Based Screen Reveals that Sulfasalazine Inhibits Tetrahydrobiopterin Biosynthesis. Nat. Chem. Biol. 7, 375-383 (2011).
15 Ameta, S. & Jäschke, A. An RNA Catalyst that Reacts with a Mechanistic Inhibitor of Serine Proteases. Chem. Sci. 4, 957-964 (2013).
16 Sharma, A. K. et al. Fluorescent RNA Labeling Using Self-Alkylating Ribozymes. ACS. Chem. Biol. doi:10.1021/cb5002119, (2014).
17 Armitage, B. A. Imaging of RNA in Live Cells. Curr. Opin. Chem. Biol. 15, 806-812 (2011).
18 Baker, M. RNA Imaging In Situ. Nat. Methods 9, 787-790 (2012).
19 Walker, S. C., Scott, F. H., Srisawat, C. & Engelke, D. R. RNA Affinity Tags for the Rapid Purification and Investigation of RNAs and RNA-Protein Complexes. Methods Mol. Biol. 488, 23-40 (2008).
20 McHugh, C. A., Russell, P. & Guttman, M. Methods for Comprehensive Experimental Identification of RNA-Protein Interactions. Genome Biol. 15, 203-212 (2014).
21 Doudna, J. A. & Lorsch, J. R. Ribozyme Catalysis: Not Different, Just Worse. Nat. Struct. Mo. Biol. 12, 395-402 (2005)
22 Lilley, D. M. J. & Eckstein, F. Ribozymes and RNA Catalysis RSC Publishing: Cambridge, UK, 2008.
23 Chen, Y. G., Kowtoniuk, W. E., Agarwal, I., Shen, Y. & Liu, D. R. LC/MS Analysis of Cellular RNA Reveals NAD-Linked RNA. Nat. Chem. Biol. 5, 879-881 (2009).
24 Thomas, J. M. & Perrin, D. M. Active Site Labeling of G8 in the Hairpin Ribozyme: Implications for Structure and Mechanism. J. Am. Chem. Soc. 128, 16540-16545 (2006).
25 Weerapana, E., Simon, G. M. & Cravatt, B. F. Disparate Proteome Reactivity Profiles of Carbon Electrophiles. Nat. Chem. Biol. 4, 405-407 (2008).
26 Barglow, K. T. & Cravatt, B. F. Discovering Disease-Associated Enzymes by Proteome Reactivity Profiling. Chem. Biol. 11, 1523-1531 (2004).
27 Simon, G. M. & Cravatt, B. F. Activity-Based Proteomics of Enzyme Superfamilies: Serine Hydrolases as a Case Study. J. Biol. Chem. 285, 11051-11055 (2010).
28 Kawarabayasi, Y. et al. Complete Genome Sequence of an Aerobic Hyper-Thermophilic Crenarchaeon, Aeropyrum Pernix K1. DNA Res. 6, 83-101 (1999)
29 Lee, N., Bessho, Y., Wei, K., Szostak, J. W. & Suga, H. Nat. Struct. Biol. 7, 28-33 (2000).
30 Sengle, G. et al. Novel RNA Catalysts for the Michael Reaction. Chem. Biol. 8, 459-473 (2001).
31 Fusz, S., Eisenftihr, A., Srivatsan, S. G., Heckel, A. & Famulok, M. A Ribozyme for the Aldol Reaction. Chem. Biol. 12, 941-950 (2005).
32 Saran, D., Nickens, D. G. & Burke, D. H. A Trans Acting Ribozyme that Phosphorylates Exogenous RNA. Biochemistry 44, 15007-15016 (2005).
33 Boysen, G., Pachkowski, B. F., Nakamura, J. & Swenberg, J. A. The Formation and Biological Significance of N7-Guanine Adducts. Mutat. Res. 678, 76-94 (2009).
34 Hansen, M. R. & Hurley, L. H. Acc. Chem. Res. Pluramycins. Old Drugs Having Modern Friends in Structural Biology. 29, 249-258 (1996).
35 Zuker, M. Mfold Web Server for Nucleic Acid Folding and Hybridization Prediction. Nucleic Acids Res. 31, 3406-3415 (2003).
36 Machanick, P. & Bailey, T. L. MEME-ChIP: Motif Analysis of Large DNA Datasets. Bioinformatics 27, 1696-1697 (2011).
37 Bao, G., Rhee, W. J. & Tsourkas, A. Fluorescent Probes for Live-Cell RNA Detection. Annu. Rev. Biomed. Eng. 11, 25-47 (2009).
38 Jao, C. Y. & Salic, A. Exploring RNA Transcription and Turnover In Vivo by Using Click Chemistry. Proc. Natl. Acad. Sci. U.S.A. 105, 15779-15784 (2008).
39 Trcek, T., Larson, D. R., Moldón, A., Query, C. C. & Singer, R. H. Single Molecule mRNA Decay Measurements Reveal Promoter-Regulated mRNA Stability in Yeast. Cell 147, 1484-1497 (2011).
40 Rabani, M. et al. Metabolic Labeling of RNA Uncovers Principles of RNA Production and Degradation Dynamics in Mammalian Cells. Nat. Biotechnol. 29, 436-442 (2011).
41 Cosma, M. P. Daughter-Specific Repression of Saccharomyces Cerevisiae HO: Ash1 is the Commander. EMBO Rep. 5, 953-957 (2004).
42 Langer, P. R., Waldrop, A. A. & Ward, D. C. Enzymatic Synthesis of Biotin-Labeled Polynucleotides: Novel Nucleic Acid Affinity Probes. Proc. Natl. Acad. Sci. U.S.A. 78, 6633-6637 (1981).
43 Curtis, E. A. & Liu, D. R. Discovery of Widespread GTP-Binding Motifs in Genomic DNA and RNA. Chem. Biol. 20, 521-532 (2013).
44 Tretyakova, N. Y., Sangaiah, R., Yen, T. Y., Swenberg, J. A. Synthesis, Characterization, and In Vitro Quantitation of N-7-Guanine Adducts of Diepoxybutane. Chem. Res. Toxicol. 10, 779-785 (1997).
45 Ponchon, L., Dardel, F. Recombinant RNA Technology: the tRNA Scaffold. Nature Meth. 4, 571-576 (2007).
46 Paul, C. P. et al. Localized Expression of Small RNA Inhibitors in Human Cells. Mol. Ther. 7, 237-247 (2003).
47 Paige, J. S., Wu, K. Y., Jaffrey, S. R. RNA Mimics of Green Fluorescent Protein. Science 333, 642-646 (2011).
48 Ghaemmaghami, et al. Global Analysis of Protein Expression in Yeast. Nature 425, 737-741 (2003).
49 Curtis, E. A. & Liu, D. R. Discovery of Widespread GTP-Binding Motifs in Genomic DNA and RNA. Chem. Biol. 20, 521-532 (2013).
50 Curtis, E. A. & Liu, D. R. A Naturally Occurring, Noncanonical GTP Aptamer Made of Simple Tandem Repeats. RNA Biol. 11, 1-10, (2014)
51 Zuker, M. Mfold Web Server for Nucleic Acid Folding and Hybridization Prediction. Nucleic Acids Res. 31, 3406-3415 (2003).
52 Machanick, P. & Bailey, T. L. MEME-ChIP: Motif Analysis of Large DNA Datasets. Bioinformatics 27, 1696-1697 (2011).
53 Pyle, A. M. Metal Ions in the Structure and Function of RNA. J. Biol. Inorg. Chem. 7, 679-690 (2002).
54 Boysen, G., Pachkowski, B. F., Nakamura, J. & Swenberg, J. A. The Formation and Biological Significance of N7-Guanine Adducts. Mutat. Res. 678, 76-94 (2009).
55 Neagu, I., Koivisto, P., Neagu, C., Kostiainen, R., Stenby, K. & Peltonen, K. Butadiene Monoxide and Deoxyguanosine Alkylation Products at the N7-Position. Carcinogenesis 16, 1809-1813 (1995).
56 Hansen, M. R. & Hurley, L. H. Acc. Chem. Res. Pluramycins. Old Drugs Having Modern Friends in Structural Biology. 29, 249-258 (1996).
57 Burns, N. Z., Witten, M. R., Jacobsen, E. N. Dual Catalysis in Enantioselective Oxidopyrylium-Based [5+2] Cycloaddition. J. Am. Chem. Soc. 133, 14578-14581 (2011).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. An isolated reactive nucleic acid comprising a reactive G nucleotide (G*) in a 5′-WGAG*RN₄₋₃₀NAGGC[U/T]CR-3′ (SEQ ID NO: 1) nucleotide sequence, wherein
R represents A or G;
W represents A or [U/T];
[U/T] represents U or T; and
N represents any nucleotide.

2. The nucleic acid of claim 1, wherein the nucleic acid comprises a stem-loop structure.

3. The nucleic acid of claim 1, wherein the nucleic acid comprises a stem-bulge structure.

4. The nucleic acid of claim 1, wherein the nucleic acid comprises ribonucleotides.

5. (canceled)

6. The nucleic acid of claim 1, wherein the nucleic acid comprises deoxyribonucleotides.

7-10. (canceled)

11. The nucleic acid of claim 1, wherein N₄₋₃₀ represents a nucleotide sequence forming a stem-loop structure.

12-13. (canceled)

14. The nucleic acid of claim 1, wherein the nucleotide at the 5′-end of N₄₋₃₀ is a G or a C and forms a G-C or a C-G base pair with the nucleotide at the 3′-end of N₄₋₃₀.

15. The nucleic acid of claim 1, wherein the third nucleotide from the 5′-end of N₄₋₃₀ is a G or a C and forms a G-C or a C-G base pair with the third nucleotide from the 3′-end of N₄₋₃₀.

16. The nucleic acid of claim 1, wherein N₄₋₃₀ comprises a 5′-GCCC[U/T]N₁₀AGGGC-3′ sequence (SEQ ID NO: 2).

17. The nucleic acid of claim 1, wherein the nucleic acid comprises a 5′-WGAG*GGCCC[U/T]N₁₋₃G[U/T]A[U/T]GRAAGGGC[U/T]AGGC[U/T]C-3′ sequence (SEQ ID NO: 3).

18. The nucleic acid of claim 1, wherein the nucleic acid comprises a 5′-GGCAAAGAG*GGCCC[U/T]N₁₋₃G[U/T]A[U/T]GRAAGGGC[U/T]AGGC [U/T]CR[U/T][U/T]G[U/T]-3′ sequence (SEQ ID NO: 4); a 5′-GGCAAAGAG*GGCCC[U/T]GGGG[U/T]A[U/T]GGAAGGGC[U/T]AGGC [U/T]CG[U/T][U/T]G[U/T]-3′ sequence (SEQ ID NO: 5); or a 5′-GGCAAAGAG*GGCCC[U/T]AG[U/T]A[U/T]GAAAGGGC[U/T]AGGC[U/T]CA[U/T][U/T]G[U/T]-3′ sequence (SEQ ID NO: 6).

19-20. (canceled)

21. A fusion molecule, comprising
(a) the nucleic acid of claim 1; and
(b) a heterologous molecule conjugated to the nucleic acid of (a).

22-35. (canceled)

36. The fusion molecule of claim 21, wherein the reactive G nucleotide (G*) is covalently bound to an electrophilic moiety.

37. The fusion molecule of claim 36, wherein the electrophilic moiety is a disubstituted epoxide.

38-39. (canceled)

40. The fusion molecule of claim 37, wherein the epoxide comprises or consists of a structure of Formula (F): 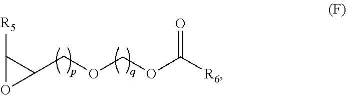
wherein p is an integer between 1 and 10, inclusive; q is an integer between 1 and 10, inclusive; R⁵ and/or R⁶ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{T}; —CO₂R^{T}; —CN; —SCN; —SR^{T}; —SOR^{T}; —SO₂R^{T}; —NO₂; —N(R^{T})₂; —NHC(O)R^{T}; or —C(R^{T})₃; wherein each occurrence of R^{T} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

41-50. (canceled)

51. A method for generating a fusion molecule, the method comprising conjugating the reactive nucleic acid molecule of claim 1 to a heterologous molecule.

52-56. (canceled)

57. A method of detecting a molecule of interest, the method comprising
(a) providing a fusion molecule comprising
(i) the molecule of interest; and
(ii) a nucleic acid conjugated to the molecule of interest of (i), wherein the nucleic acid comprises a reactive G nucleotide (G*) in a 5′-WGAG*RN₄₋₃₀NAGGC[U/T]CR-3′ (SEQ ID NO: 1) nucleotide sequence,
wherein
R represents A or G;
W represents A or [U/T];
[U/T] represents U or T; and
N represents any nucleotide;
(b) contacting the molecule of (a) with a disubstituted epoxide comprising a detectable label under conditions suitable for the disubstituted epoxide to form a covalent bond with the reactive G nucleotide (G*); and
(c) detecting the detectable label bound to the molecule of interest.

58-105. (canceled)

106. An electrophilic probe capable of forming a covalent bond with a reactive RNA comprising the consensus sequence of SEQ ID NO: 1, wherein the probe comprises a disubstituted epoxide.

107-108. (canceled)

109. The electrophilic probe of claim 106, wherein the epoxide comprises or consists of a structure of Formula (F): 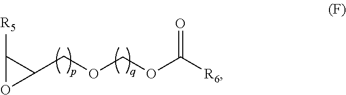
wherein p is an integer between 1 and 10, inclusive; q is an integer between 1 and 10, inclusive; R⁵ and/or R⁶ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —C(═O)R^{T}; —CO₂R^{T}; —CN; —SCN; —SR^{T}; —SOR^{T}; —SO₂R^{T}; —NO₂; —N(R^{T})₂; —NHC(O)R^{T}; or —C(R^{T})₃; wherein each occurrence of R^{T} is independently hydrogen, a protecting group, a reactive handle, such as a click chemistry handle, aliphatic, heteroaliphatic, acyl, aryl, heteroaryl, alkoxy, aryloxy, alkylthio, arylthio, amino, alkylamino, dialkylamino, heteroaryloxy, or heteroarylthio.

110-119. (canceled)

120. An expression construct comprising
(a) a nucleic acid sequence encoding a reactive nucleic acid; and
(b) a cloning site for inserting a heterologous nucleic acid sequence encoding a gene product of interest to generate a hybrid nucleic acid sequence encoding a fusion of the nucleic acid sequence of (a) and the gene product of interest.

121-125. (canceled)


SEQUENCE LISTING (34 sequences)

SEQ ID NO: 1 - DNA, 42 bp, Artificial Sequence
  Synthetic Polynucleotide
  misc_feature (4)..(4): Reactive G nucleotide
  misc_feature (6)..(35): n is a, c, g, t or u
  misc_feature (10)..(35): These nucleotides may be absent
  misc_feature (40)..(40): n is u or t
wgagrnnnnnnnnnnnnnnnnnnnnnnnnnnnnnnaggcncr

SEQ ID NO: 2 - DNA, 20 bp, Artificial Sequence
  Synthetic Polynucleotide
  misc_feature (5)..(5): n is u or t
  misc_feature (6)..(15): n is a, c, g, t or u
gcccnnnnnnnnnnnagggc

SEQ ID NO: 3 - DNA, 32 bp, Artificial Sequence
  Synthetic Polynucleotide
  misc_feature (4)..(4): Reactive G nucleotide
  misc_feature (10)..(10): n is u or t
  misc_feature (11)..(13): n is a, c, g, t or u
  misc_feature (12)..(13): These nucleotides may be absent
  misc_feature (15)..(15): n is u or t
  misc_feature (17)..(17): n is u or t
  misc_feature (26)..(26): n is u or t
  misc_feature (31)..(31): n is u or t
wgagggcccnnnngnangraagggcnaggcnc

SEQ ID NO: 4 - DNA, 42 bp, Artificial Sequence
  Synthetic Polynucleotide
  misc_feature (9)..(9): Reactive G nucleotide
  misc_feature (15)..(15): n is u or t
  misc_feature (16)..(18): n is a, c, g, t or u
  misc_feature (17)..(18): These nucleotides may be absent
  misc_feature (20)..(20): n is u or t
  misc_feature (22)..(22): n is u or t
  misc_feature (31)..(31): n is u or t
  misc_feature (36)..(36): n is u or t
  misc_feature (39)..(40): n is u or t
  misc_feature (42)..(42): n is u or t
ggcaaagagggcccnnnngnangraagggcnaggcncrnngn

SEQ ID NO: 5 - DNA, 42 bp, Artificial Sequence
  Synthetic Polynucleotide
  misc_feature (9)..(9): Reactive G nucleotide
  misc_feature (15)..(15): n is u or t
  misc_feature (20)..(20): n is u or t
  misc_feature (22)..(22): n is u or t
  misc_feature (31)..(31): n is u or t
  misc_feature (36)..(36): n is u or t
  misc_feature (39)..(40): n is u or t
  misc_feature (42)..(42): n is u or t
ggcaaagagggcccnggggnanggaagggcnaggcncgnngn

SEQ ID NO: 6 - DNA, 40 bp, Artificial Sequence
  Synthetic Polynucleotide
  misc_feature (9)..(9): Reactive G nucleotide
  misc_feature (15)..(15): n is u or t
  misc_feature (18)..(18): n is u or t
  misc_feature (20)..(20): n is u or t
  misc_feature (29)..(29): n is u or t
  misc_feature (34)..(34): n is u or t
  misc_feature (37)..(38): n is u or t
  misc_feature (40)..(40): n is u or t
ggcaaagagggcccnagnangaaagggcnaggcncanngn

SEQ ID NO: 7 - RNA, 48 nt, Artificial Sequence
  Synthetic Polynucleotide
ggccgcuccagaagagggccccuucgggggcuaggcucgaugucggcc

SEQ ID NO: 8 - protein, 18 aa, Artificial Sequence
  Synthetic Polypeptide
GGSGGSGGSGGSGGSGGS

SEQ ID NO: 9 - protein, 16 aa, Artificial Sequence
  Synthetic Polypeptide
SGSETPGTSESATPES

SEQ ID NO: 10 - protein, 12 aa, Artificial Sequence
  Synthetic Polypeptide
SGSETPGTSESA

SEQ ID NO: 11 - protein, 21 aa, Artificial Sequence
  Synthetic Polypeptide
SGSETPGTSESATPEGGSGGS

SEQ ID NO: 12 - protein, 15 aa, Artificial Sequence
  Synthetic Polypeptide
VPFLLEPDNINGKTC

SEQ ID NO: 13 - protein, 12 aa, Artificial Sequence
  Synthetic Polypeptide
GSAGSAAGSGEF

SEQ ID NO: 14 - protein, 12 aa, Artificial Sequence
  Synthetic Polypeptide
SIVAQLSRPDPA

SEQ ID NO: 15 - protein, 10 aa, Artificial Sequence
  Synthetic Polypeptide
MKIIEQLPSA

SEQ ID NO: 16 - protein, 10 aa, Artificial Sequence
  Synthetic Polypeptide
VRHKLKRVGS

SEQ ID NO: 17 - protein, 12 aa, Artificial Sequence
  Synthetic Polypeptide
GHGTGSTGSGSS

SEQ ID NO: 18 - protein, 7 aa, Artificial Sequence
  Synthetic Polypeptide
MSRPDPA

SEQ ID NO: 19 - protein, 4 aa, Artificial Sequence
  Synthetic Polypeptide
GGSM

SEQ ID NO: 20 - DNA, 36 bp, Artificial Sequence
  Synthetic Polynucleotide
  misc_feature (6)..(6): n is u or t
  misc_feature (7)..(30): n is a, c, g, t or u
  misc_feature (9)..(30): These nucleotides may be absent
  misc_feature (36)..(36): n is a, c, g, t or u
rgcccnnnnnnnnnnnnnnnnnnnnnnnnnagggcn

SEQ ID NO: 21 - DNA, 6 bp, Artificial Sequence
  Synthetic Polynucleotide
  misc_feature (6)..(6): n is u or t
rgcccn

SEQ ID NO: 22 - DNA, 6 bp, Artificial Sequence
  Synthetic Polynucleotide
  misc_feature (6)..(6): n is a, c, g, t or u
agggcn

SEQ ID NO: 23 - DNA, 21 bp, Artificial Sequence
  Synthetic Polynucleotide
gccgcgaattcactagtgatt

SEQ ID NO: 24 - DNA, 20 bp, Artificial Sequence
  Synthetic Polynucleotide
taggccgcgggaattcgatt

SEQ ID NO: 25 - DNA, 18 bp, Artificial Sequence
  Synthetic Polynucleotide
aatcactagtgaattcgc

SEQ ID NO: 26 - DNA, 42 bp, Artificial Sequence
  Synthetic Polynucleotide
  misc_feature (10)..(10): n is a, c, g or t
ggcaaagagngccctggggtatggaagggctaggctcgttgt

SEQ ID NO: 27 - RNA, 111 nt, Artificial Sequence
  Synthetic Polynucleotide
gcccggauagcucagucgguagagcagcggccgcuccagaagagggccccuucgggggcuaggcucgaugucggccgcggguccaggguucaagucccuguucgggcgcca

SEQ ID NO: 28 - RNA, 332 nt, Artificial Sequence
  Synthetic Polynucleotide
gcccggauagcucagucgguagagcagcggccgaaucuacuuagaggccggauucuccagaagagggccccuucgggggcuaggcucgauguaauccggccgcaggucgacucuagaaacggauacuuagaggccggauucuccagaagagggccccuucgggggcuaggcucgauguaauccggccgcaggucgacucuagaaagucuuacuuagaggccggauucuccagaagagggccccuucgggggcuaggcucgauguaauccggccgcaggucgacucuagaaacuuaccgcggguccaggguucaagucccuguucgggcgcca

SEQ ID NO: 29 - RNA, 41 nt, Artificial Sequence
  Synthetic Polynucleotide
gcaaagagggcccugggguauggaagggcuaggcucguugu

SEQ ID NO: 30 - DNA, 63 bp, Artificial Sequence
  Synthetic Polynucleotide
taacaaggttctagaggcaaagagggccctagtatgaaagggctaggctcattgtggacagca

SEQ ID NO: 31 - DNA, 65 bp, Aeropyrum pernix
taacaaggttctggaggcaaagagggccctggggtatggaagggctaggctcgttgtggacagta

SEQ ID NO: 32 - RNA, 67 nt, Artificial Sequence
  Synthetic Polynucleotide
gguaacaagguucuggaggcaaagagggcccugggguauggaagggcuaggcucguuguggacagua

SEQ ID NO: 33 - RNA, 42 nt, Artificial Sequence
  Synthetic Polynucleotide
ggcaaagagggcccugggguauggaagggcuaggcucguugu

SEQ ID NO: 34 - DNA, 13 bp, Artificial Sequence
  Synthetic Polynucleotide
  misc_feature (1)..(1): n is a or u
ngagruaggcucr